SEQUENCE LISTING (14 sequences)

SEQ ID NO: 1 - protein, 5 aa, synthetic construct
  REGION 1..5: note = Synthetic Sequence
NYWMS

SEQ ID NO: 2 - protein, 17 aa, synthetic construct
  REGION 1..17: note = Synthetic Sequence
DIHGNRGFNYHASWAKG

SEQ ID NO: 3 - protein, 9 aa, synthetic construct
  REGION 1..9: note = Synthetic Sequence
ADDSGSHDI

SEQ ID NO: 4 - protein, 12 aa, synthetic construct
  REGION 1..12: note = Synthetic Sequence
QSSQSVYSAKLS

SEQ ID NO: 5 - protein, 7 aa, synthetic construct
  REGION 1..7: note = Synthetic Sequence
YGSTLAS

SEQ ID NO: 6 - protein, 12 aa, synthetic construct
  REGION 1..12: note = Synthetic Sequence
QGTFYGPDWYFA

SEQ ID NO: 7 - protein, 116 aa, synthetic construct
  REGION 1..116: note = Synthetic Sequence
EVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSS

SEQ ID NO: 8 - protein, 111 aa, synthetic construct
  REGION 1..111: note = Synthetic Sequence
DIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIK

SEQ ID NO: 9 - protein, 446 aa, synthetic construct
  REGION 1..446: note = Synthetic Sequence
EVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 218 aa, synthetic construct
  REGION 1..218: note = Synthetic Sequence
DIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 446 aa, synthetic construct
  REGION 1..446: note = Synthetic Sequence
QQQLVESGGRLVTPGGSLTLTCTASGFSLNNYWMSWVRQAPGKGLEWIGDIHGNRGFNYHASWAKGRFTVSRTSTTVDLRMTSLTTEDTAIYFCARADDSGSHDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 218 aa, synthetic construct
  REGION 1..218: note = Synthetic Sequence
ELDLTQTPSPVSAAVGDTVTINCQSSQSVYSAKLSWYQQKPGQPPKLLIYYGSTLASGVPSRFKGSGSGTQFSLTISDVQCADAATYYCQGTYYGPDWYFAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 446 aa, synthetic construct
  REGION 1..446: note = Synthetic Sequence
QQQLVESGGRLVTPGGSLTLTCTASGFSLNNYWMSWVRQAPGKGLEWIGDIHGNRGFNYHASWAKGRFTVSRTSTTVDLRMTSLTTEDTAIYFCARADDSGSHDIWGPGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPMVTCVVVDVSEDDPDVQISWFVNNVEVLTAQTQTHREDYNSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 14 - protein, 218 aa, synthetic construct
  REGION 1..218: note = Synthetic Sequence
ELDLTQTPSPVSAAVGDTVTINCQSSQSVYSAKLSWYQQKPGQPPKLLIYYGSTLASGVPSRFKGSGSGTQFSLTISDVQCADAATYYCQGTYYGPDWYFAFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2025/0057958 A1
(43) Pub. Date: Feb. 20, 2025

(54) CYTOTOXICITY TARGETING CHIMERAS FOR CCR2-EXPRESSING CELLS

(71) Applicant: GLAXOSMITHKLINE INTELLECTUAL PROPERTY DEVELOPMENT LIMITED, Brentford (GB)

(72) Inventors: Peiling Chen, Collegeville, PA (US); Michael Gerard Darcy, Collegeville, PA (US); Jason W. Dodson, Collegeville, PA (US); Beth Anne Knapp-Reed, Collegeville, PA (US); Joseph Paul Marino, JR., Collegeville, PA (US); Jeffrey Alan Oplinger, Collegeville, PA (US); Matthew Robert Sender, Collegeville, PA (US); Brandon James Turunen, Boston, MA (US); Guosen Ye, Collegeville, PA (US); Cunyu Zhang, Collegeville, PA (US)

(21) Appl. No.: 18/802,386

(22) Filed: Aug. 13, 2024

Related U.S. Application Data
(63) Continuation of application No. PCT/IB2023/051750, filed on Feb. 24, 2023.
(60) Provisional application No. 63/314,099, filed on Feb. 25, 2022.

(51) Int. Cl. A61K 47/54

(52) U.S. Cl. CPC A61K 47/545

(57) ABSTRACT

The present disclosure relates to heterobifunctional molecules, referred to as cytotoxicity targeting chimeras (CyTaCs) or antibody recruiting molecules (ARMs) that are able to simultaneously bind a target cell-surface protein as well as an exogenous antibody protein. The present disclosure also relates to agents capable of binding to a receptor on a surface of a pathogenic cell and inducing the depletion of the pathogenic cell in a subject for use in the treatment of cancer, inflammatory diseases, autoimmune diseases, viral infection, or bacterial infection.

### CROSS-REFERENCE TO RELATED APPLICATIONS

This application is a continuation of International Patent Application No. PCT/IB2023/051750, filed Feb. 24, 2023, which claims priority to U.S. Patent Application No. 63/314,099 filed on Feb. 25, 2022; the contents of each of which are incorporated by reference herein in their entirety.

### REFERENCE TO AN ELECTRONIC SEQUENCE LISTING

This application contains a Sequence Listing which has been submitted electronically via Patent Center in XML format and is hereby incorporated by reference in its entirety. Said XML copy, created on Aug. 12, 2024, is named 211808_seqlist.xml and is 20,538 bytes in size.

### FIELD OF THE DISCLOSURE

The present disclosure relates to heterobifunctional molecules, referred to as cytotoxicity targeting chimeras (CyTaCs) or antibody recruiting molecules (ARMs) that are able to simultaneously bind a target cell-surface protein as well as an exogenous antibody protein. The present disclosure also relates to agents capable of binding to a receptor on a surface of a pathogenic cell and inducing the depletion of the pathogenic cell in a subject for use in the treatment of cancer, inflammatory diseases, autoimmune diseases, viral infection, or bacterial infection.

### BACKGROUND

Cell-surface proteins and their ligands play key roles in a range of inflammatory, infectious, and autoimmune diseases as well tumor initiation, growth and metastasis. Antibody-based therapeutics have promising properties as drug candidates for these indications due to their selectivity for pathogenic cell-surface targets and their ability to direct immune surveillance to target-expressing tissues or cells to induce depletion of the pathogenic cells. Examples of such depletion mechanisms include antibody-dependent cellular cytotoxicity (ADCC), antibody-dependent cellular phagocytosis (ADCP), and complement-dependant cytotoxicity (CDC). However, antibody-based therapeutics often suffer from a lack of bioavailability, high cost, thermal instability, and difficult manufacturing due to their size, complexity and peptide based structures. Conversely, small molecule therapeutics often provide affordability, stability, and the convenience of oral dosing, but may suffer from poor selectivity and off-target effects, while also lacking the immune control of therapeutic antibodies.

Accordingly, a need exists for improved therapeutic approaches that target pathogenic cells for use in the treatment of disease. Such compositions and related methods are provided in the present disclosure.

### SUMMARY

In one aspect, the present disclosure provides a heterobifunctional molecule referred to as a cytoxicity targeting chimera (CyTaC) or an antibody recruiting molecule (ARM), wherein the ARM comprises a moiety that binds a target cell-surface protein on a cell and a moiety that binds an exogenous antibody. In a further aspect, the ARM comprises a divalent linker that links the target-binding moiety to the antibody-binding moiety. In a further aspect, the target-binding moiety is a C—C chemokine receptor type 2 (CCR2)-binding moiety. In a further aspect, the exogenous antibody is an anti-cotinine antibody, or antigen-binding fragment thereof.

In a further aspect, the ARM is a compound of Formula (I):

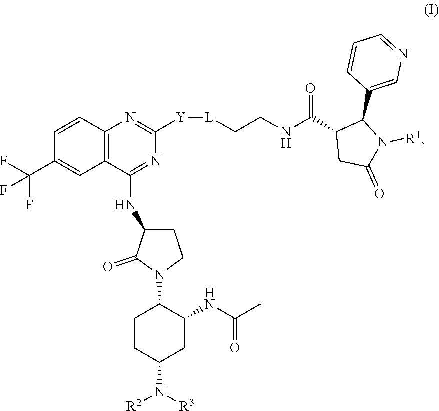

or a pharmaceutically acceptable salt thereof,
wherein:
R¹ is C₁₋₄ alkyl or C₃₋₆ cycloalkyl;
R² is hydrogen or C₁₋₄ alkyl;
R³ is hydrogen or C₁₋₄ alkyl;
Y is a bond or a divalent spacer moiety of one to twelve atoms in length; and
L is a divalent linker as described herein.

In one aspect, the present disclosure provides a method of treating and/or preventing a disease or disorder in a patient in need thereof, comprising: administering to the patient a compound of Formula (I) as disclosed herein and an anti-cotinine antibody, or antigen-binding fragment thereof.

In one aspect, the present disclosure provides a method of increasing antibody-dependent cell cytotoxicity (ADCC) of CCR2-expressing cells comprising: contacting the cells with a compound of Formula (I) as disclosed herein and an anti-cotinine antibody, or antigen-binding fragment thereof.

In one aspect, the present disclosure provides a method of depleting CCR2-expressing cells comprising: contacting the cells with a compound of Formula (I) as disclosed herein and an anti-cotinine antibody, or antigen-binding fragment thereof.

In one aspect, the present disclosure provides a compound of Formula (I) as disclosed herein for use in therapy. In a further aspect, the present disclosure provides a combination comprising a compound of Formula (I) as disclosed herein and an anti-cotinine antibody, or antigen-binding fragment thereof, for use in therapy.

In one aspect, the present disclosure provides a combination comprising a compound of Formula (I) as disclosed herein and an anti-cotinine antibody, or antigen-binding fragment thereof, for use in the treatment of a disease or disorder.

In one aspect, the present disclosure provides use of a compound of Formula (I) as disclosed herein in the manufacture of a medicament for the treatment of a disease or disorder. In a further aspect, the present disclosure provides use of a combination comprising a compound of Formula (I) as disclosed herein and an anti-cotinine antibody, or antigen-binding fragment thereof, in the manufacture of a medicament for the treatment of a disease or disorder.

In one aspect, the present disclosure provides a combination comprising a compound of Formula (I) as disclosed herein and an anti-cotinine antibody, or antigen-binding fragment thereof.

### BRIEF DESCRIPTION OF THE FIGURES

FIG. 1: Schematic representation of cytotoxicity targeting chimeras (CyTaCs) technology compared to current antibody technology.

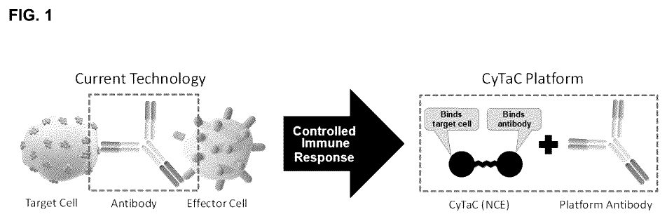

### DETAILED DESCRIPTION

In one aspect, the present disclosure provides a compound of Formula (I):

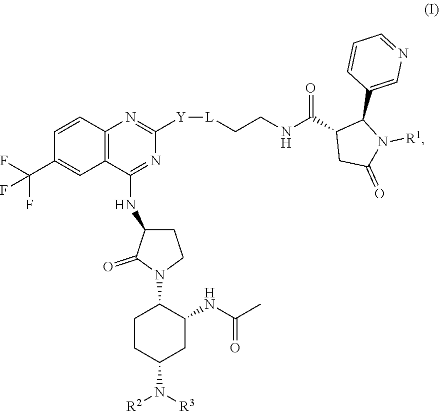

or a pharmaceutically acceptable salt thereof,
wherein:
R¹ is C₁₋₄ alkyl or C₃₋₆ cycloalkyl;
R² is hydrogen or C₁₋₄ alkyl;
R³ is hydrogen or C₁₋₄ alkyl;
Y is a bond or a divalent spacer moiety of one to twelve atoms in length; and
L is a divalent linker of Formula (L-a), (L-b), (L-c), (L-d), (L-e), (L-f), (L-g), (L-h), (L-i), (L-j), (L-k), (L-m), (L-n-i), (L-n-ii), (L-n-iii), or (L-n-iv).

In one embodiment of the disclosure Lisa divalent linker of Formula (L-a):

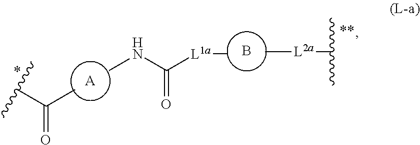

or a stereoisomer thereof,
wherein:
Ring A and Ring B are each independently C₄₋₆ cycloalkylene;
L^{1a} is C₃₋₅ linear alkylene, wherein 1 or 2 methylene units are replaced with —O— or —NR^{a}—;
each R^{a} is independently hydrogen or C₁₋₃ alkyl; and
L^{2a} is —O—, —NHC(O)—, or —CH₂—O—;
wherein

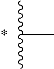

represents a covalent bond to the Y group of Formula (I), or when Y is a bond, a covalent bond to the quinazoline group of Formula (I), and

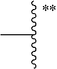

represents a covalent bond to the methylene group of Formula (I).

In another embodiment, Ring A and Ring B of Formula (L-a) are each independently

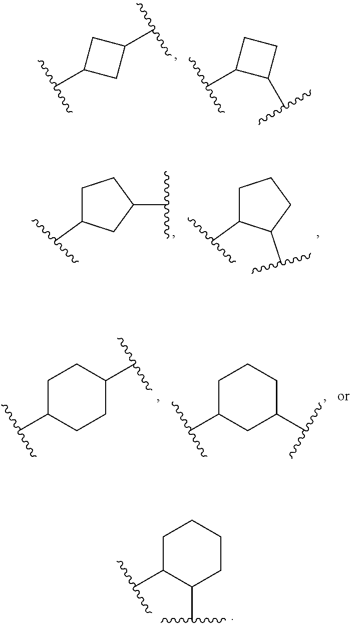

In another embodiment, L is a divalent linker of Formula (L-a-i):

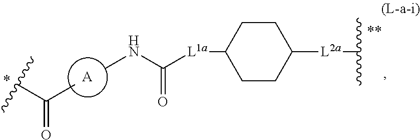

or a stereoisomer thereof,
wherein:
Ring A is C₄₋₆ cycloalkylene;
L^{1a} is C₃₋₅ linear alkylene, wherein 1 or 2 methylene units are replaced with —O— or —NR^{a}—;
each R^{a} is independently hydrogen or C₁₋₃ alkyl; and
L^{2a} is —O—, —NHC(O)—, or —CH₂—O—;
wherein

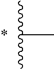

represents a covalent bond to the Y group of Formula (I), or when Y is a bond, a covalent bond to the quinazoline group of Formula (I), and

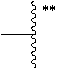

represents a covalent bond to the methylene group of Formula (I).

In another embodiment, Ring A of Formula (L-a-i) is

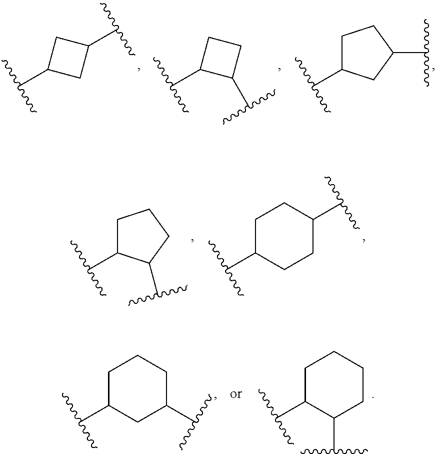

In another embodiment, L is a divalent linker of Formula (L-a-ii):

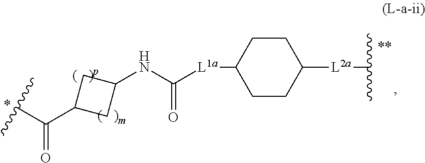

or a stereoisomer thereof,
wherein:
L^{1a} is C₃₋₅ linear alkylene, wherein 1 or 2 methylene units are replaced with —O— or —NR^{a}—;
each R^{a} is independently hydrogen or C₁₋₃ alkyl;
L^{2a} is —O—, —NHC(O)—, or —CH₂—O—;
p is 1 or 2; and
m is 1 or 2;
wherein

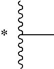

represents a covalent bond to the Y group of Formula (I), or when Y is a bond, a covalent bond to the quinazoline group of Formula (I), and

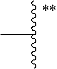

represents a covalent bond to the methylene group of Formula (I).

In another embodiment, L^{1a} of Formula (L-a), (L-a-i), or (L-a-ii) is selected from

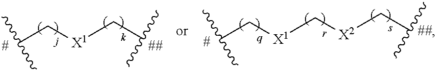

wherein:

j is 1, 2, 3, or 4;
k is 0, 1, 2, or 3;
the sum of j and k is 2, 3, or 4;
q is 1 or 2;
r is 1 or 2;
s is 0 or 1;
the sum of q, r, and s is 2 or 3;
X¹ and X² are independently —O— or NR^{a}; and
each R^{a} is independently hydrogen or C₁₋₃ alkyl;
wherein

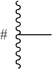

represents a covalent bond to the C(O) group of Formula (L-a), (L-a-i), or (L-a-ii), and

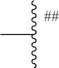

represents a covalent bond to Ring B of Formula (L-a) or to the cyclohexylene group of Formula (L-a-i) or (L-a-ii).

In another embodiment, L^{1a} of Formula (L-a), (L-a-i), or (L-a-ii) is selected from —(CH₂)₂O—, —(CH₂)₃O—, —(CH₂)₄O—, —(CH₂)₂OCH₂—, —(CH₂)₃OCH₂—, —(CH₂)₂O(CH₂)₂—, —CH₂OCH₂—, —CH₂O(CH₂)₂—, —CH₂O(CH₂)₃—, —CH₂OCH₂O—, or —CH₂OCH₂OCH₂—. In another embodiment, L^{1a} of Formula (L-a), (L-a-i), or (L-a-ii) is selected from —(CH₂)₂O—, —(CH₂)₃O—, —(CH₂)₂OCH₂—, or —(CH₂)₃OCH₂—. In another embodiment, L^{1a} of Formula (L-a), (L-a-i), or (L-a-ii) is selected from —(CH₂)₂NR^{a}—, —(CH₂)₃NR^{a}—, —(CH₂)₄NR^{a}—, —(CH₂)₂NR^{a}CH₂—, —(CH₂)₃NR^{a}CH₂—, —(CH₂)₂NR^{a}(CH₂)₂—, —CH₂NR^{a}CH₂—, —CH₂NR^{a}(CH₂)₂—, —CH₂NR^{a}(CH₂)₃—, —CH₂NR^{a}CH₂NR^{a}—, or —CH₂NR^{a}CH₂NR^{a}CH₂—, wherein each R^{a} is independently hydrogen or C₁₋₃ alkyl. In another embodiment, L^{1a} of Formula (L-a), (L-a-i), or (L-a-ii) is selected from —(CH₂)₂NR^{a}—, —(CH₂)₃NR^{a}—, —(CH₂)₂NR^{a}CH₂—, or —(CH₂)₃NR^{a}CH₂—, wherein R^{a} is hydrogen or C₁₋₃ alkyl. In another embodiment, L^{1a} of Formula (L-a), (L-a-i), or (L-a-ii) is selected from —(CH₂)₂NH—, —(CH₂)₃NH—, —(CH₂)₄NH—, —(CH₂)₂NHCH₂—, —(CH₂)₃NHCH₂—, —(CH₂)₂NH(CH₂)₂—, —CH₂NHCH₂—, —CH₂NH(CH₂)₂—, —CH₂NH(CH₂)₃—, —CH₂NHCH₂NH—, or —CH₂NHCH₂NHCH₂—. In another embodiment, L^{1a} of Formula (L-a), (L-a-i), or (L-a-ii) is selected from —(CH₂)₂NH—, —(CH₂)₃NH—, —(CH₂)₂NHCH₂—, or —(CH₂)₃NHCH₂—. In another embodiment, L^{1a} of Formula (L-a), (L-a-i), or (L-a-ii) is selected from —CH₂OCH₂NR^{a}—, —CH₂NR^{a}CH₂O—, —CH₂OCH₂NR^{a}CH₂—, —CH₂NR^{a}CH₂OCH₂—, wherein R^{a} is independently hydrogen or C₁₋₃ alkyl. In another embodiment, L^{1a} of Formula (L-a), (L-a-i), or (L-a-ii) is selected from —CH₂OCH₂NH—, —CH₂NHCH₂O—, —CH₂OCH₂NHCH₂—, —CH₂NHCH₂OCH₂—.

In another embodiment, L is a divalent linker of Formula (L-a-iii):

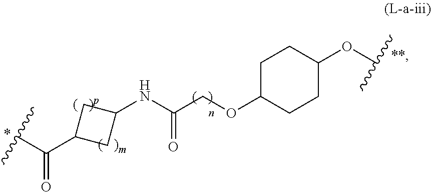

or a stereoisomer thereof,
wherein:
p is 1 or 2;
m is 1 or 2; and
n is 1, 2, or 3;
wherein

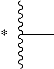

represents a covalent bond to the Y group of Formula (I), or when Y is a bond, a covalent bond to the quinazoline group of Formula (I), and

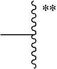

represents a covalent bond to the methylene group of Formula (I).

In another embodiment, L is a divalent linker of Formula (L-a) selected from the group consisting of:

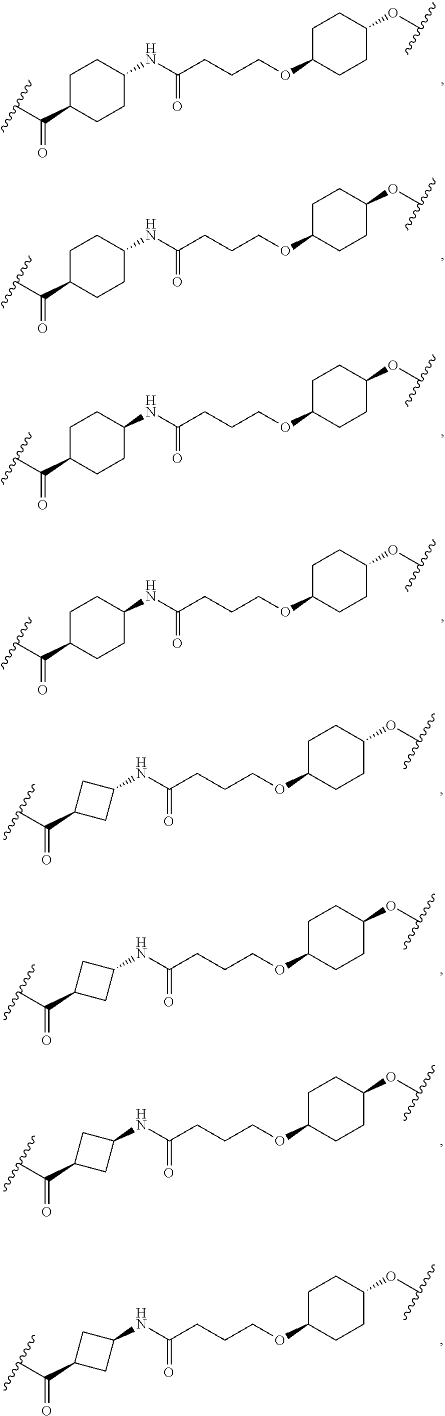

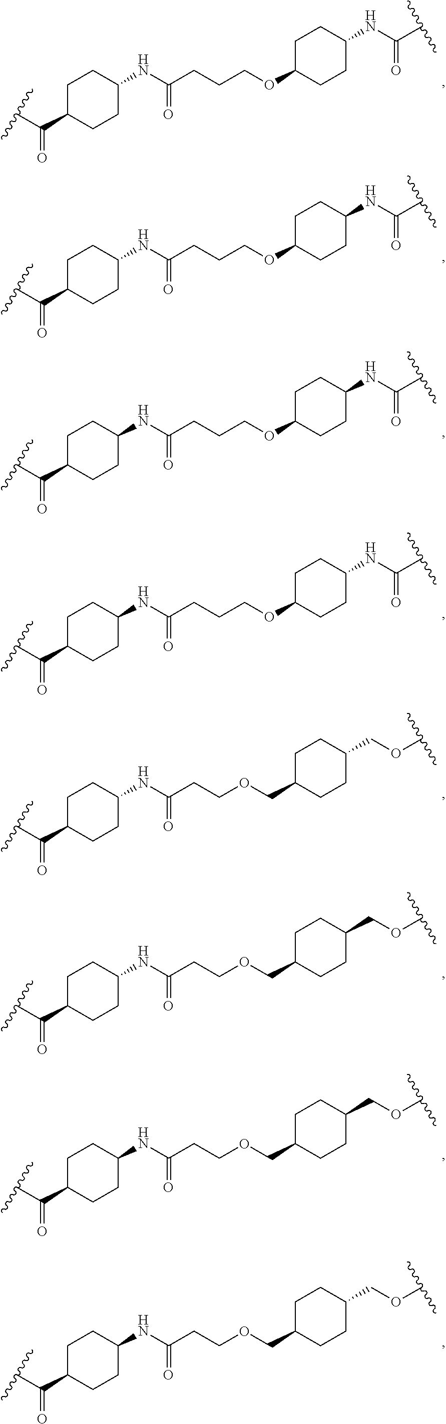

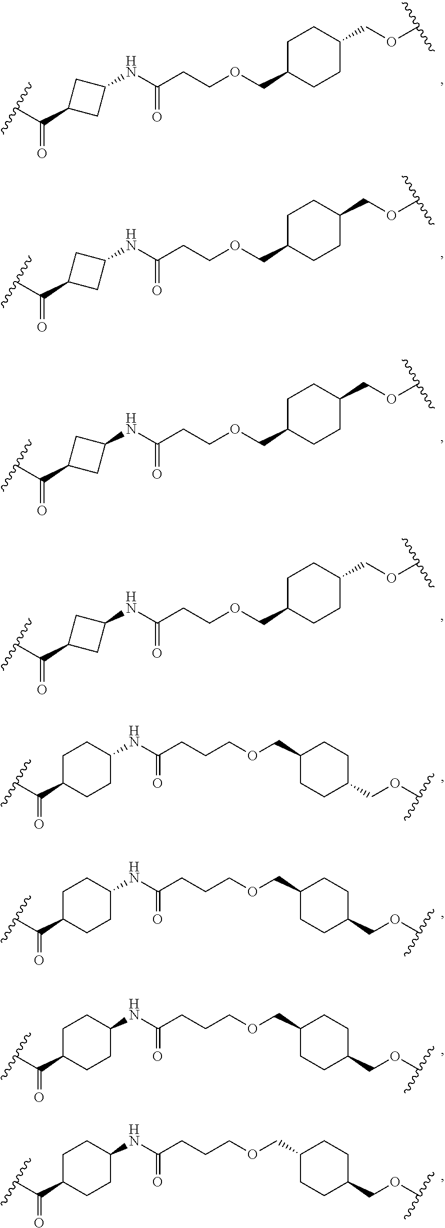

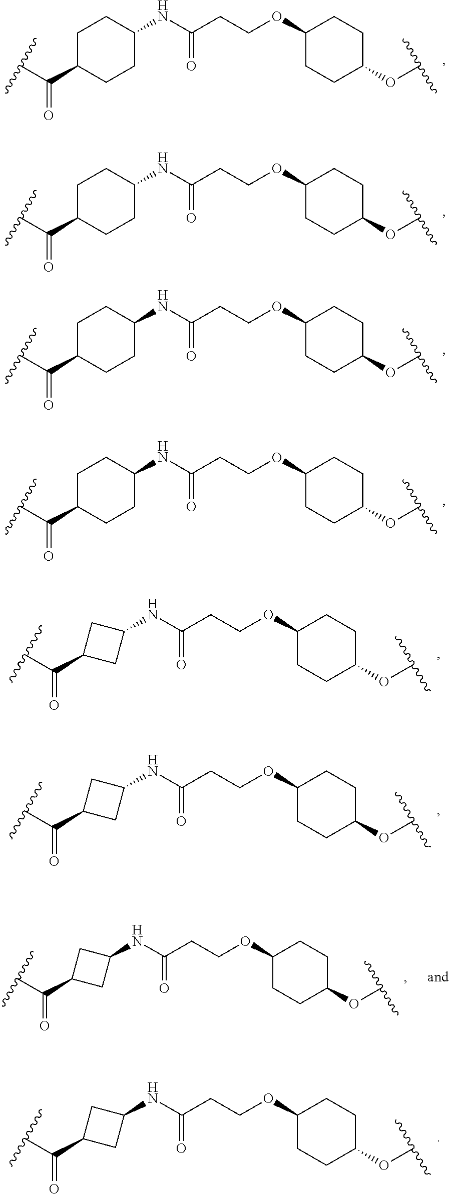

In another embodiment, L is a divalent linker of Formula (L-b):

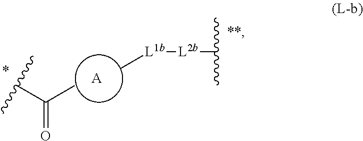

or a stereoisomer thereof,
wherein:
Ring A is C₄₋₆ cycloalkylene or C₇₋₉ bridged bicyclic cycloalkylene;
L^{1b} is —CH₂—NH—C(O)—, —NHC(O)—, or —C(O)NH—;
L^{2b} is C₆₋₁₂ linear alkylene, wherein 1, 2, 3, or 4 methylene units are replaced with —O—, —NR^{1b}—, —C(O)NR^{1b}—, or —NR^{1b}C(O)—; or

### L^{2b} is

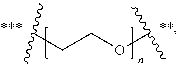

wherein n is 1, 2, 3, or 4, and

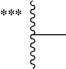

represents a covalent bond to L^{1b}; and
each R^{1b} is independently hydrogen or C₁₋₃ alkyl;
wherein

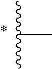

represents a covalent bond to the Y group of Formula (I), or when Y is a bond, a covalent bond to the quinazoline group of Formula (I), and

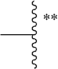

represents a covalent bond to the methylene group of Formula (I).

In another embodiment, Ring A of Formula (L-b) is

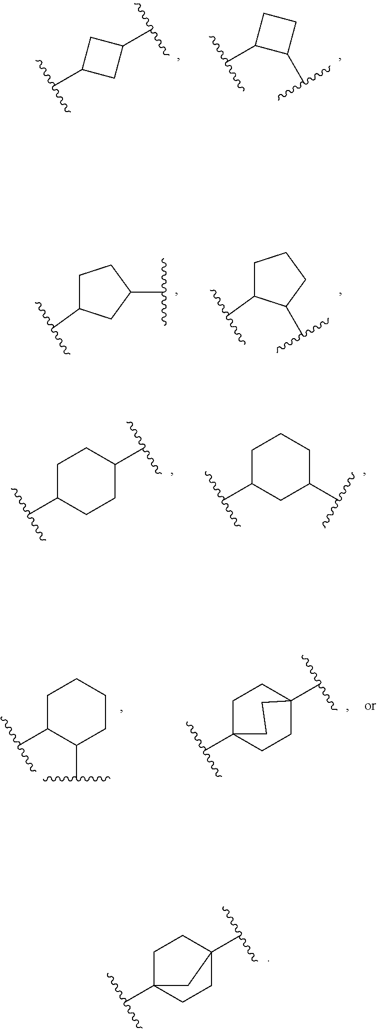

In another embodiment, L is a divalent linker of Formula (L-b-i):

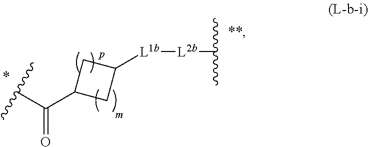

or a stereoisomer thereof,
wherein:
L^{1b} is —CH₂—NH—C(O)—, —NHC(O)—, or —C(O)NH—;
L^{2b} is C₆₋₁₂ linear alkylene, wherein 1, 2, 3, or 4 methylene units are replaced with —O—, —NR^{1b}—, —C(O)NR^{1b}—, or —NR^{1b}C(O)—; or

### L^{2b} is

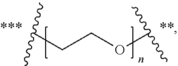

wherein n is 1, 2, 3, or 4, and

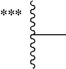

represents a covalent bond to L^{1b};
each R^{1b} is independently hydrogen or C₁₋₃ alkyl;
p is 1 or 2; and
m is 1 or 2;
wherein

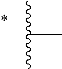

represents a covalent bond to the Y group of Formula (I), or when Y is a bond, a covalent bond to the quinazoline group of Formula (I), and

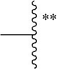

represents a covalent bond to the methylene group of Formula (I).

In another embodiment, L^{2b} of Formula (L-b) or (L-b-i) is selected from

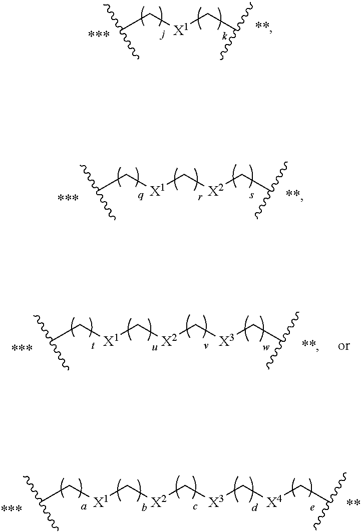

wherein:
j is 1, 2, 3, 4, 5, 6, 7, 8, 9, or 10;
k is 1, 2, 3, 4, 5, 6, 7, 8, 9, or 10;
the sum of j and k is 5, 6, 7, 8, 9, 10, or 11;
q is 1, 2, 3, 4, 5, 6, 7, 8, or 9;
r is 1, 2, 3, 4, 5, 6, 7, 8, or 9;
s is 0, 1, 2, 3, 4, 5, 6, 7, or 8;
the sum of q, r, and s is 4, 5, 6, 7, 8, 9, or 10;
t is 1, 2, 3, 4, 5, 6, or 7;
u is 1, 2, 3, 4, 5, 6, or 7;
vis 1, 2, 3, 4, 5, 6, or 7;
w is 0, 1, 2, 3, 4, 5, or 6;
the sum of t, u, v, and w is 3, 4, 5, 6, 7, 8, or 9;
a is 1, 2, 3, 4, or 5;
b is 1, 2, 3, 4, or 5;
c is 1, 2, 3, 4, or 5;
d is 1, 2, 3, 4, or 5;
e is 0, 1, 2, 3, or 4;
the sum of a, b, c, d, and e is 4, 5, 6, 7, or 8;
X¹, X², X³, and X⁴ are independently —O—, —NR^{1b}—, —C(O)NR^{1b}—, or —NR^{1b}C(O)—; and
each R^{1b} is independently hydrogen or C₁₋₃ alkyl;
wherein

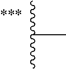

represents a covalent bond to L^{1b} of Formula (L-b) or (L-b-i), and

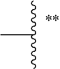

represents a covalent bond to the methylene group of Formula (I).

In another embodiment, L is a divalent linker of Formula (L-b) selected from the group consisting of:

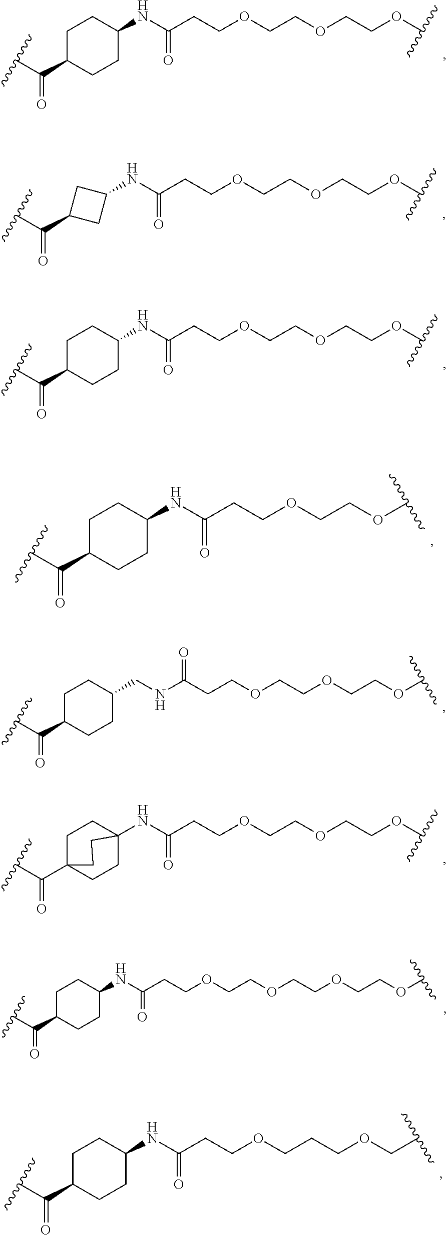

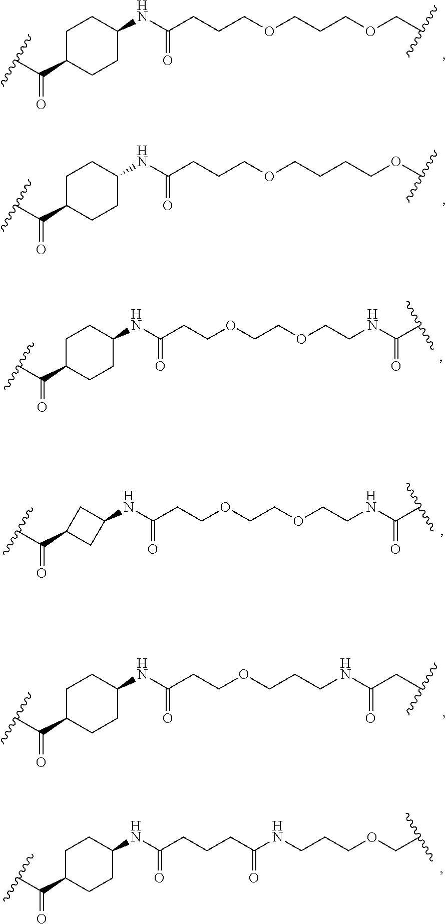 [SMILES: *COCCCOCCCC(=O)N[C@H]1CC[C@@H](C(*)=O)CC1 ; *OCCCCOCCCC(=O)N[C@H]1CC[C@H](C(*)=O)CC1 ; *C(=O)NCCOCCOCCC(=O)N[C@H]1CC[C@@H](C(*)=O)CC1 ; *C(=O)NCCOCCOCCC(=O)N[C@H]1C[C@@H](C(*)=O)C1 ; *CC(=O)NCCCOCCC(=O)N[C@H]1CC[C@@H](C(*)=O)CC1 ; *COCCCNC(=O)CCCC(=O)N[C@H]1CC[C@@H](C(*)=O)CC1]

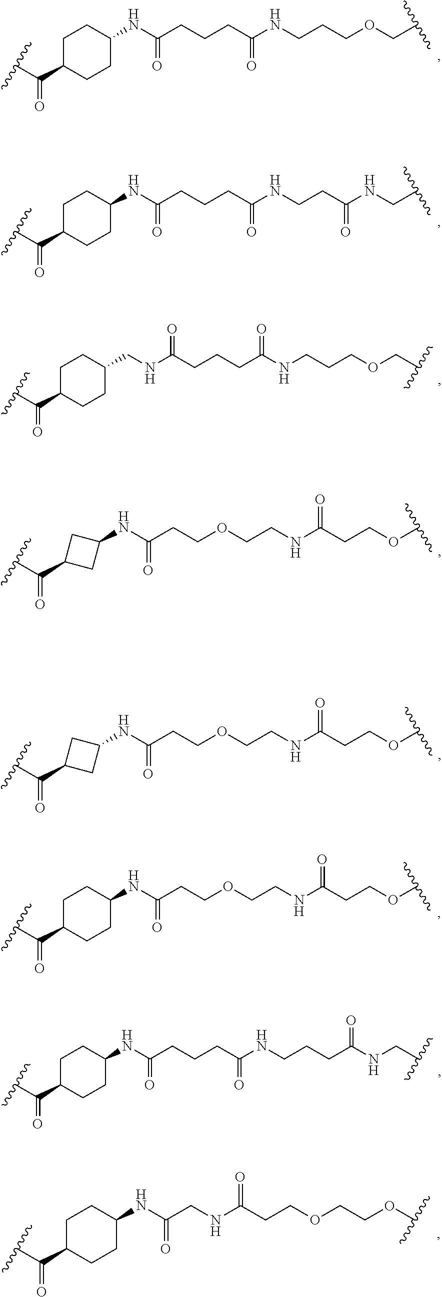

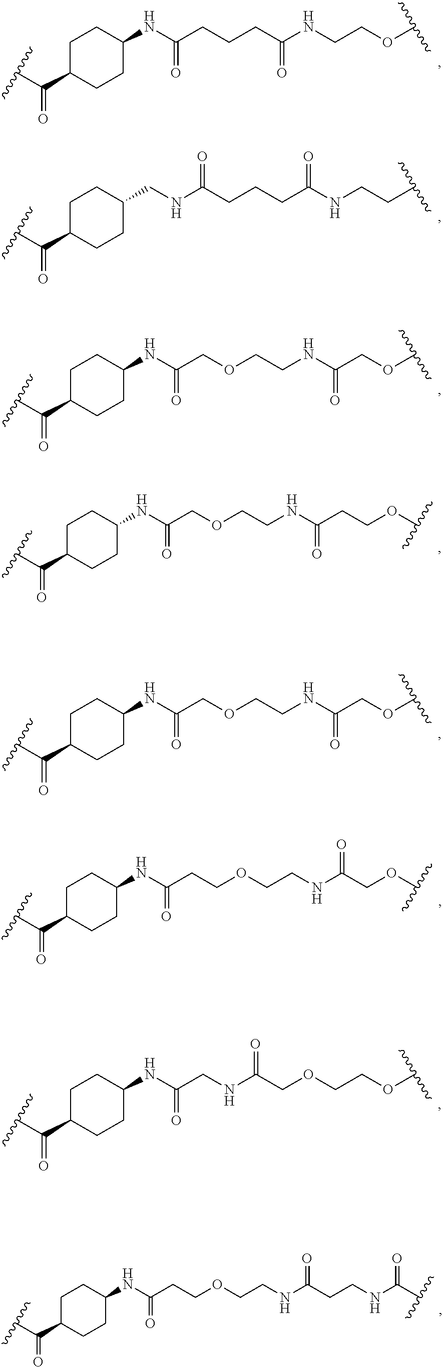

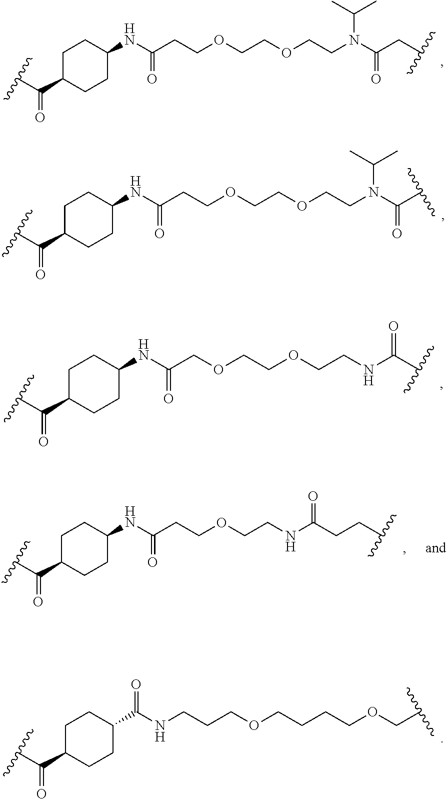 [SMILES: *CC(=O)N(CCOCCOCCC(=O)N[C@H]1CC[C@@H](C(*)=O)CC1)C(C)C ; *C(=O)N(CCOCCOCCC(=O)N[C@H]1CC[C@@H](C(*)=O)CC1)C(C)C ; *C(=O)NCCOCCOCC(=O)N[C@H]1CC[C@@H](C(*)=O)CC1 ; *CCC(=O)NCCOCCC(=O)N[C@H]1CC[C@@H](C(*)=O)CC1 ; *COCCCCOCCCNC(=O)[C@H]1CC[C@H](C(*)=O)CC1]

In another embodiment, L is a divalent linker of Formula (L-c):

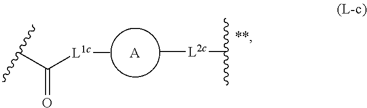

or a stereoisomer thereof,
wherein:
L^{1c} is C₂₋₁₀ linear alkylene, wherein 1, 2, or 3 methylene units are replaced with —O—, —NH—, —NHC(O)—, or —C(O)NH—;
Ring A is C₄₋₆ cycloalkylene or C₇₋₉ bridged bicyclic cycloalkylene; and
L^{2c} is —O— or a saturated C₂₋₁₀ linear alkylene, wherein 1, 2, or 3 methylene units are replaced with —O—, —NH—, —NHC(O)—, or —C(O)NH—;
wherein

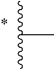

represents a covalent bond to the Y group of Formula (I), or when Y is a bond, a covalent bond to the quinazoline group of Formula (I), and

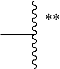

represents a covalent bond to the methylene group of Formula (I).

In another embodiment, Ring A of Formula (L-c) is

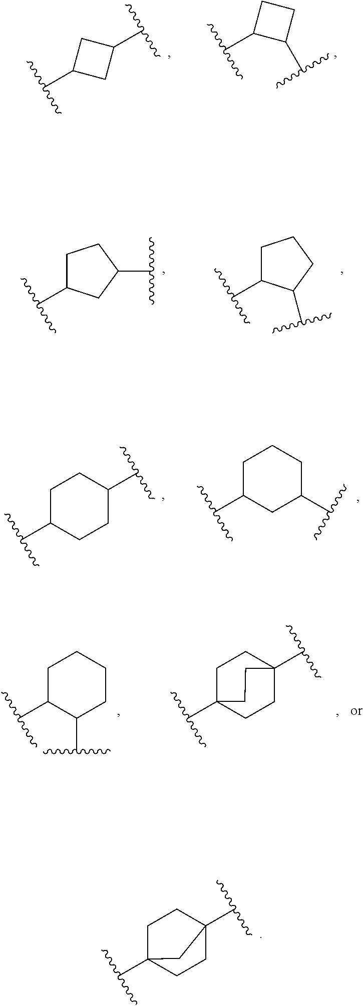

In another embodiment, L is a divalent linker of Formula (L-c-i):

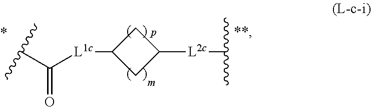

or a stereoisomer thereof,
wherein:
L^{1c} is C₂₋₁₀ linear alkylene, wherein 1, 2, or 3 methylene units are replaced with —O—, —NH—, —NHC(O)—, or —C(O)NH—;
L^{2c} is —O— or a saturated C₂₋₁₀ linear alkylene, wherein 1, 2, or 3 methylene units are replaced with —O—, —NH—, —NHC(O)—, or —C(O)NH—;
p is 1 or 2; and
m is 1 or 2;
wherein

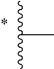

represents a covalent bond to the Y group of Formula (I), or when Y is a bond, a covalent bond to the quinazoline group of Formula (I), and

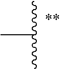

represents a covalent bond to the methylene group of Formula (I).

In another embodiment, L^{1c} of Formula (L-c) or (L-c-i) is selected from

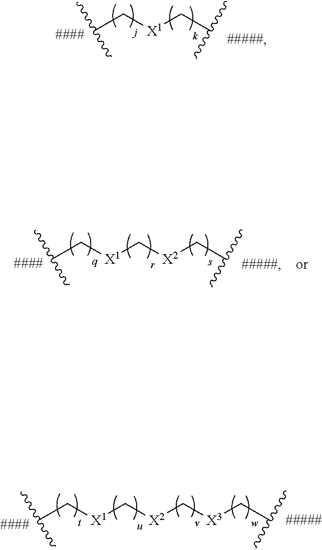

wherein:
j is 1, 2, 3, 4, 5, 6, 7, 8, or 9;
k is 0, 1, 2, 3, 4, 5, 6, 7, or 8;
the sum of j and k is 1, 2, 3, 4, 5, 6, 7, 8, or 9;
q is 1, 2, 3, 4, 5, 6, or 7;
r is 1, 2, 3, 4, 5, 6, or 7;
s is 0, 1, 2, 3, 4, 5, or 6;
the sum of q, r, and s is 2, 3, 4, 5, 6, 7, or 8;
t is 1, 2, 3, 4, or 5;
u is 1, 2, 3, 4, or 5;
v is 1, 2, 3, 4, or 5;
w is 0, 1, 2, 3, or 4;
the sum of t, u, v, and w is 3, 4, 5, 6, or 7; and
X¹, X² and X³ are independently —O—, —NH—, —NHC(O)—, or —C(O)NH—;
wherein

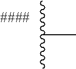

represents a covalent bond to the C(O) group of Formula (L-c) or (L-c-i), and

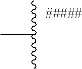

represents a covalent bond to the ring of Formula (L-c) or (L-c-i).

In another embodiment, L^{2c} of Formula (L-c) or (L-c-i) is selected from

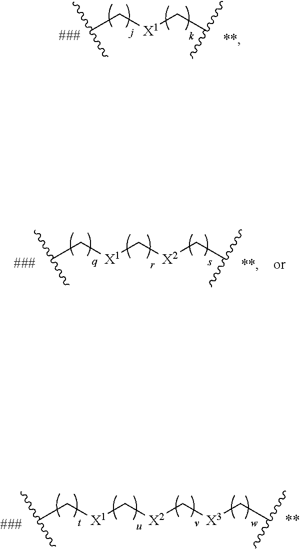

wherein:
j is 0, 1, 2, 3, 4, 5, 6, 7, 8, or 9;
k is 0, 1, 2, 3, 4, 5, 6, 7, 8, or 9;
the sum of j and k is 1, 2, 3, 4, 5, 6, 7, 8, or 9;
q is 0, 2, 3, 4, 5, 6, or 7;
r is 1, 2, 3, 4, 5, 6, 7, or 8;
s is 0, 1, 2, 3, 4, 5, 6, or 7;
the sum of q, r, and s is 1, 2, 3, 4, 5, 6, 7, or 8;
t is 0, 1, 2, 3, 4, or 5;
u is 1, 2, 3, 4, 5, or 6;
v is 1, 2, 3, 4, 5, or 6;
w is 0, 1, 2, 3, 4, or 5;
the sum of t, u, v, and w is 2, 3, 4, 5, 6, or 7; and
X¹, X² and X³ are independently —O—, —NH—, —NHC(O)—, or —C(O)NH—;
wherein

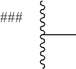

represents a covalent bond to the ring of Formula (L-c) or (L-c-i), and

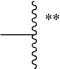

represents a covalent bond to the methylene group of Formula (I).

In another embodiment, L is a divalent linker of Formula (L-c) selected from the group consisting of:

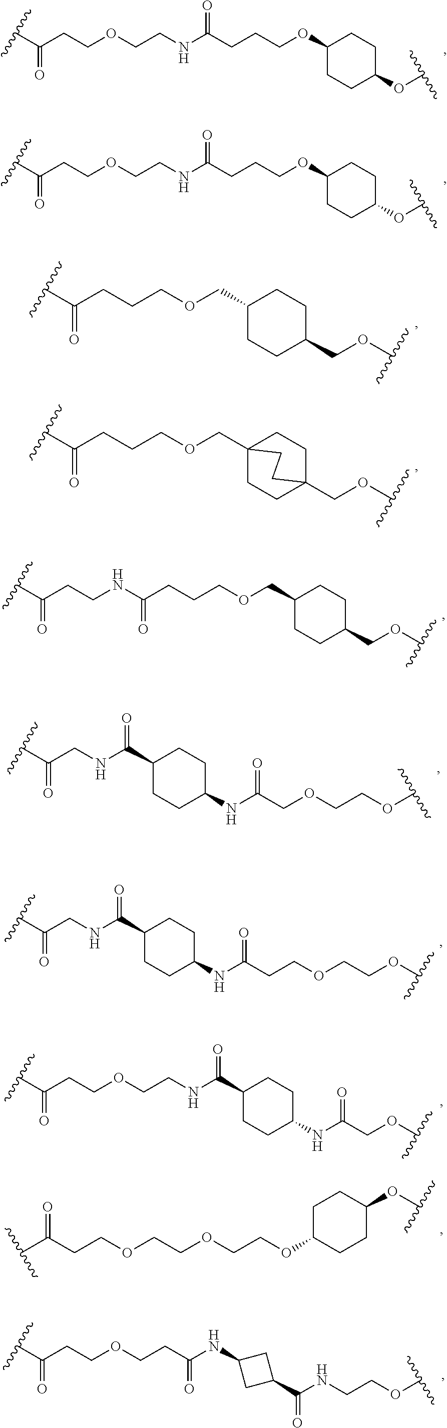

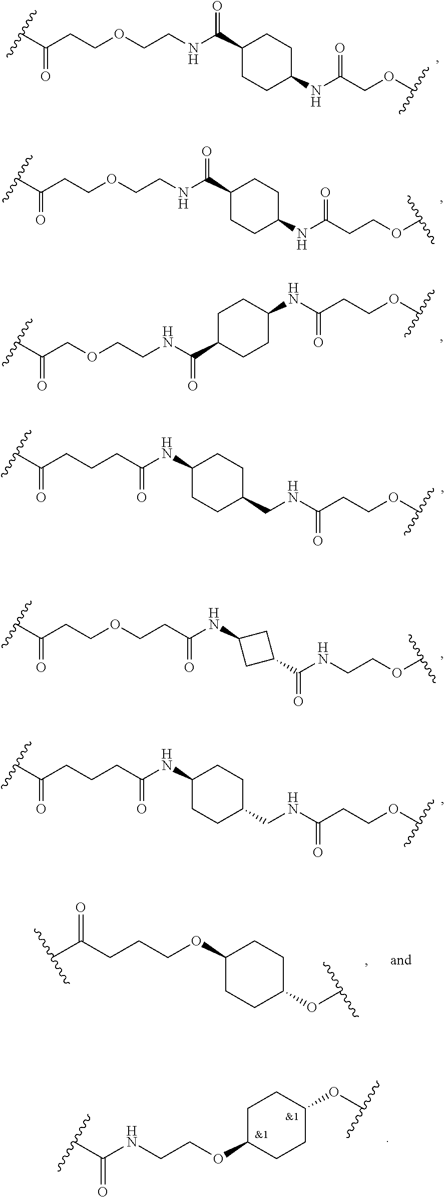

In another embodiment, L is a divalent linker of Formula (L-d):

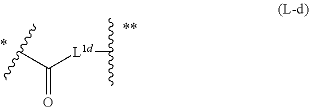

wherein:
L^{1d} is C₁₂₋₃₁ linear alkylene, wherein 1, 2, 3, 4, 5, 6, 7, 8, 9, 10, or 11 methylene units are replaced with —NH—, —O—, —C(O)NH—, —NHC(O)—, or —NHC(O)—NH—;
wherein

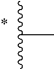

represents a covalent bond to the Y group of Formula (I), or when Y is a bond, a covalent bond to the quinazoline group of Formula (I), and

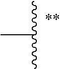

represents a covalent bond to the methylene group of Formula (I). In another embodiment, L^{1d} is a C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, or C₃₁ linear alkylene, wherein 1, 2, 3, 4, 5, 6, 7, 8, 9, 10, or 11 methylene units are replaced with —NH—, —O—, —C(O)NH—, —NHC(O)—, or —NHC(O)—NH—. In another embodiment, L^{1d} is C₁₂₋₂₂ linear alkylene, for example, C₁₂, C₁₃, C₁₄, C₁₅, C₁₆, C₁₇, C₁₈, C₁₉, C₂₀, C₂₁, or C₂₂, wherein 1, 2, 3, 4, or 5 methylene units are replaced with —NH—, —O—, —C(O)NH—, —NHC(O)—, or —NHC(O)—NH—.

In another embodiment, L^{1d} of Formula (L-d) is selected from

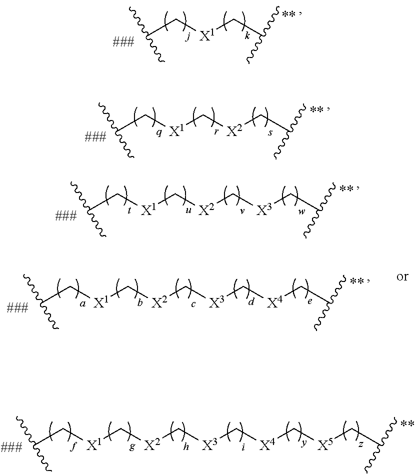

wherein:
j is 1, 2, 3, 4, 5, 6, 7, 8, 9, 10, 11, 12, 13, 14, 15, 16, 17, 18, 19, or 20;
k is 1, 2, 3, 4, 5, 6, 7, 8, 9, 10, 11, 12, 13, 14, 15, 16, 17, 18, 19, or 20;
the sum of j and k is 11, 12, 13, 14, 15, 16, 17, 18, 19, 20, or 21;
q is 1, 2, 3, 4, 5, 6, 7, 8, 9, 10, 11, 12, 13, 14, 15, 16, 17, 18, or 19;
r is 1, 2, 3, 4, 5, 6, 7, 8, 9, 10, 11, 12, 13, 14, 15, 16, 17, 18, or 19;
s is 0, 1, 2, 3, 4, 5, 6, 7, 8, 9, 10, 11, 12, 13, 14, 15, 16, 17, or 18;
the sum of q, r, and s is 10, 11, 12, 13, 14, 15, 16, 17, 18, 19, or 20;
t is 1, 2, 3, 4, 5, 6, 7, 8, 9, 10, 11, 12, 13, 14, 15, 16, or 17;
u is 1, 2, 3, 4, 5, 6, 7, 8, 9, 10, 11, 12, 13, 14, 15, 16, or 17;
v is 1, 2, 3, 4, 5, 6, 7, 8, 9, 10, 11, 12, 13, 14, 15, 16, or 17;
w is 0, 1, 2, 3, 4, 5, 6, 7, 8, 9, 10, 11, 12, 13, 14, 15, or 16;
the sum of t, u, v, and w is 9, 10, 11, 12, 13, 14, 15, 16, 17, 18, or 19;
a is 1, 2, 3, 4, 5, 6, 7, 8, 9, 10, 11, 12, 13, 14, or 15;
b is 1, 2, 3, 4, 5, 6, 7, 8, 9, 10, 11, 12, 13, 14, or 15;
c is 1, 2, 3, 4, 5, 6, 7, 8, 9, 10, 11, 12, 13, 14, or 15;
d is 1, 2, 3, 4, 5, 6, 7, 8, 9, 10, 11, 12, 13, 14, or 15;
e is 0, 1, 2, 3, 4, 5, 6, 7, 8, 9, 10, 11, 12, 13, or 14;
the sum of a, b, c, d, and e is 8, 9, 10, 11, 12, 13, 14, 15, 16, 17, or 18;
f is 1, 2, 3, 4, 5, 6, 7, 8, 9, 10, 11, 12, or 13;
g is 1, 2, 3, 4, 5, 6, 7, 8, 9, 10, 11, 12, or 13;
h is 1, 2, 3, 4, 5, 6, 7, 8, 9, 10, 11, 12, or 13;
i is 1, 2, 3, 4, 5, 6, 7, 8, 9, 10, 11, 12, or 13;
y is 1, 2, 3, 4, 5, 6, 7, 8, 9, 10, 11, 12, or 13;
z is 0, 1, 2, 3, 4, 5, 6, 7, 8, 9, 10, 11, or 12;
the sum of f, g, h, i, y, and z is 7, 8, 9, 10, 11, 12, 13, 14, 15, 16, or 17; and
X¹, X², X³, X⁴, and X⁵ are independently —NH—, —O—, —C(O)NH—, —NHC(O)—, or —NHC(O)—NH—;
wherein

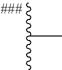

represents a covalent bond to the C(O) group of Formula (L-d), and

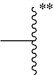

represents a covalent bond to the methylene group of Formula (I).

In another embodiment, L^{1d} of Formula (L-d) is

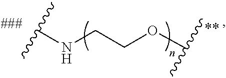

wherein n is 4, 5, 6, 7, 8, 9, or 10;
wherein

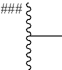

represents a covalent bond to the C(O) group of Formula (L-d), and

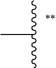

represents a covalent bond to the methylene group of Formula (I).

In another embodiment, L is a divalent linker of Formula (L-d) selected from the group consisting of:

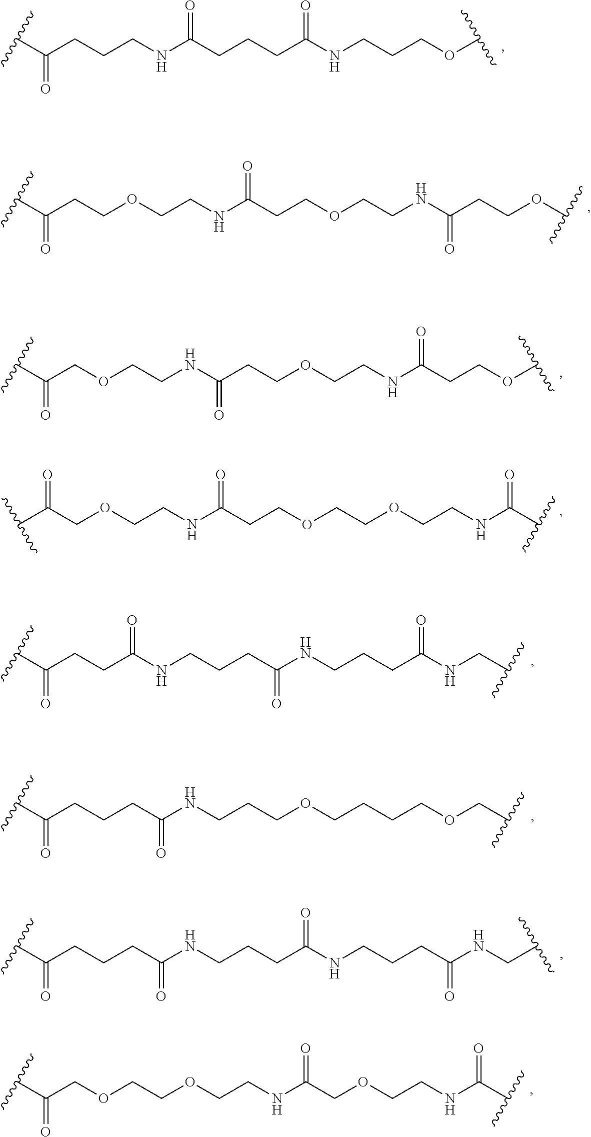

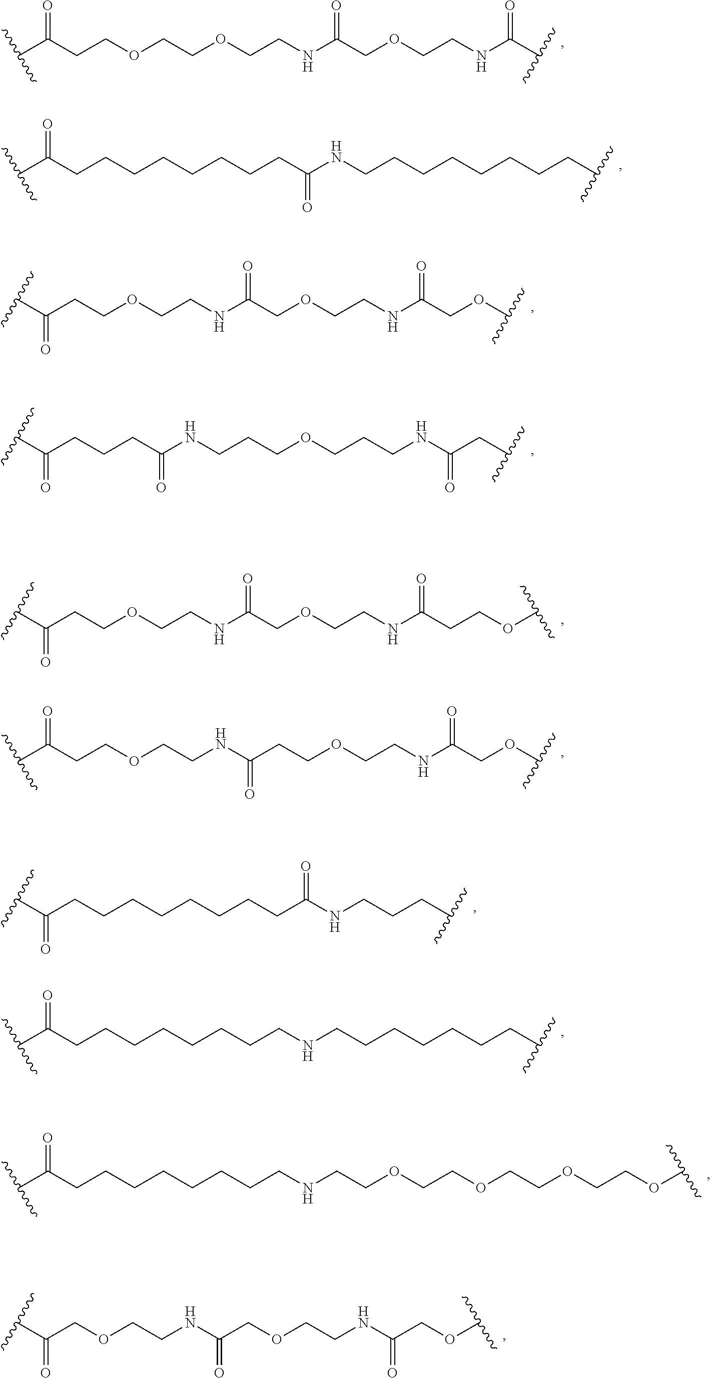

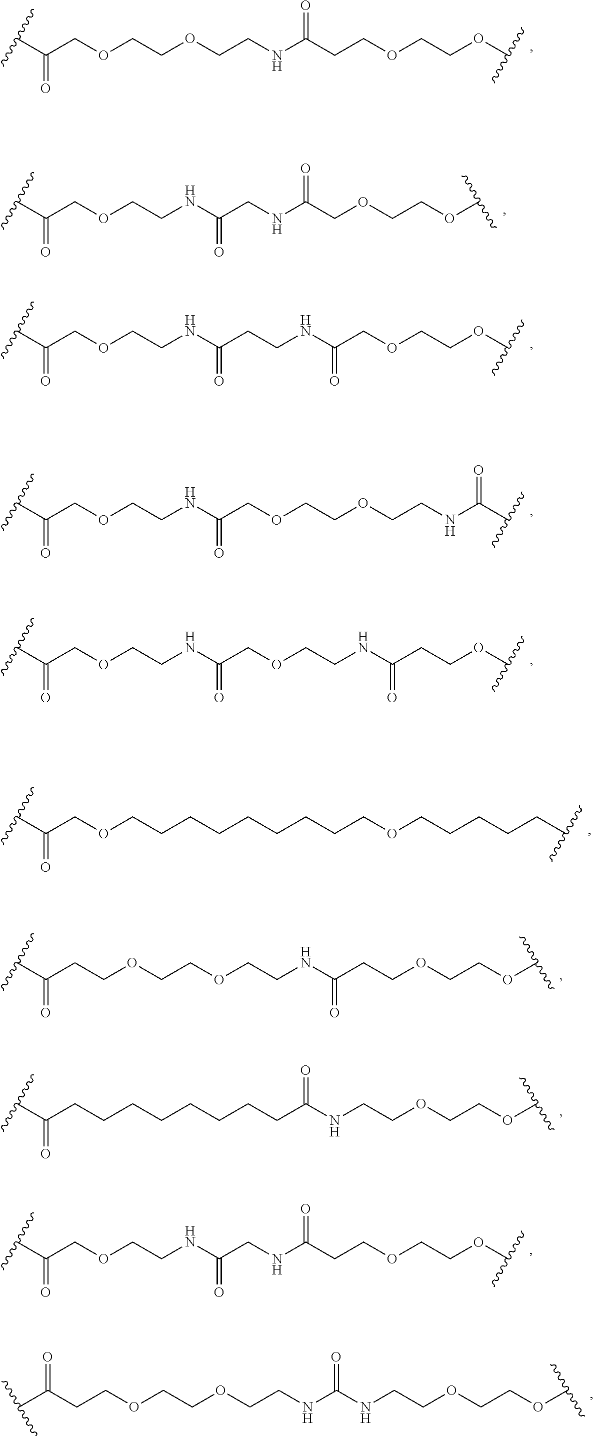

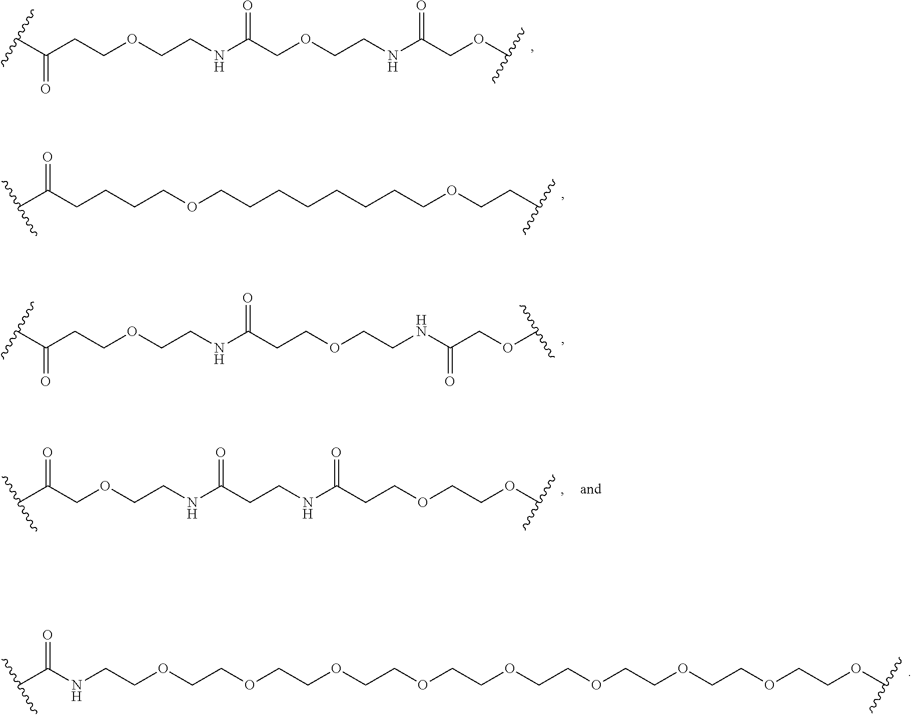 [SMILES: *OCC(=O)NCCOCC(=O)NCCOCCC(*)=O ; *CCOCCCCCCCCOCCCCC(*)=O ; *OCC(=O)NCCOCCC(=O)NCCOCCC(*)=O ; *OCCOCCC(=O)NCCC(=O)NCCOCC(*)=O ; *OCCOCCOCCOCCOCCOCCOCCOCCOCCNC(*)=O]

In another embodiment, L is a divalent linker of Formula (L-e):

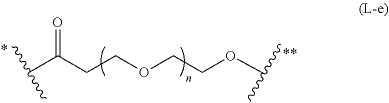

wherein:
n is an integer of 3 to 50;
wherein

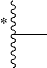

represents a covalent bond to the Y group of Formula (I), or when Y is a bond, a covalent bond to the quinazoline group of Formula (I), and

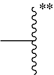

represents a covalent bond to the methylene group of Formula (I).

In another embodiment, n of Formula (L-e) is 3 to 25, 3 to 10, 3 to 8, 3 to 7, 3 to 5, or 3 to 4. In another embodiment, n of Formula (L-e) is 5 to 22, 7 to 15, or 9 to 13. In another embodiment, n of Formula (L-e) is 3, 4, 5, 7, 8, 11, 22, or 50.

In another embodiment, L is a divalent linker of Formula (L-f):

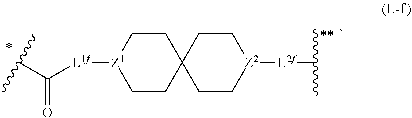

or a stereoisomer thereof,
wherein:
L^{1f} is a bond; C₁₋₆ linear alkylene, wherein 0, 1, or 2 methylene units are replaced with —O—, —NH—, or —C(O)—; or —(C₃₋₆ cycloalkylene)-NHC(O)—;
L^{2f} is a bond, —NHC(O)—, —C(O)NH—, or a C₁₋₆ linear alkylene, wherein 0, 1, or 2 methylene units are replaced with —O—; and
each of Z¹ and Z² is independently N or CH;
wherein

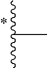

represents a covalent bond to the Y group of Formula (I), or when Y is a bond, a covalent bond to the quinazoline group of Formula (I), and

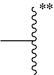

represents a covalent bond to the methylene group of Formula (I).

In another embodiment, L^{1f} of Formula (L-f) is selected from

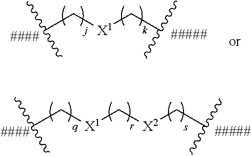

wherein:
j is 1, 2, 3, 4, or 5;
k is 0, 1, 2, 3, or 4;
the sum of j and k is 1, 2, 3, 4, or 5;
q is 1, 2, or 3;
r is 1, 2, or 3;
s is 0, 1, 2;
the sum of q, r, and s is 2, 3, or 4; and
X¹ and X² are independently —O—, —NH—, or —C(O)—; or —(C₃₋₆ cycloalkylene)-NHC(O)—;
wherein

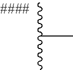

represents a covalent bond to the C(O) group of Formula (L-f), and

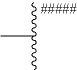

represents a covalent bond to the ring of Formula (L-f).

In another embodiment, L^{2f} of Formula (L-f) is selected from

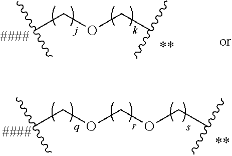

wherein:
j is 1, 2, 3, 4, or 5;
k is 0, 1, 2, 3, or 4;
the sum of j and k is 1, 2, 3, 4, or 5;
q is 1, 2, or 3;
r is 1, 2, or 3;
s is 0, 1, 2; and
the sum of q, r, and s is 2, 3, or 4;
wherein

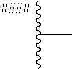

represents a covalent bond to the ring of Formula (L-f), and

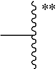

represents a covalent bond to the methylene group of Formula (I).

In another embodiment, L is a divalent linker of Formula (L-f) selected from the group consisting of:

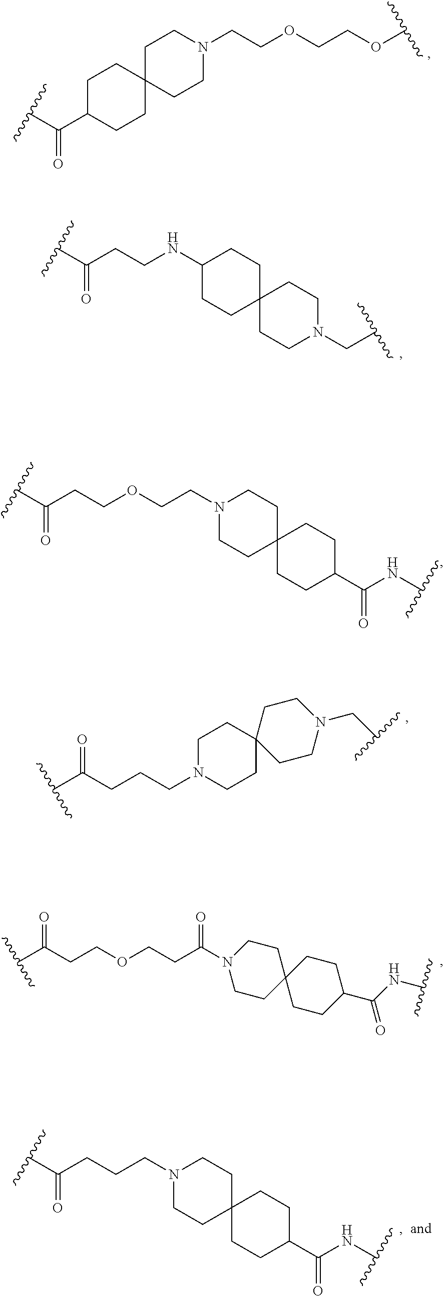 [SMILES: *OCCOCCN1CCC2(CCC(C(*)=O)CC2)CC1 ; *CN1CCC2(CCC(NCCC(*)=O)CC2)CC1 ; *NC(=O)C1CCC2(CC1)CCN(CCOCCC(*)=O)CC2 ; *CN1CCC2(CC1)CCN(CCCC(*)=O)CC2 ; *NC(=O)C1CCC2(CC1)CCN(C(=O)CCOCCC(*)=O)CC2 ; *NC(=O)C1CCC2(CC1)CCN(CCCC(*)=O)CC2]

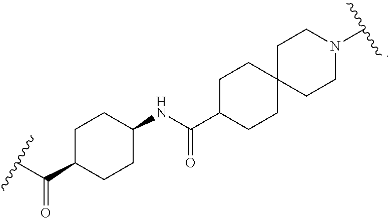 [SMILES: *C(=O)[C@H]1CC[C@@H](NC(=O)C2CCC3(CC2)CCN(*)CC3)CC1]

In another embodiment, L is a divalent linker of Formula (L-g):

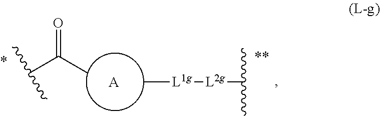

wherein:
Ring A is a 5 to 6 membered heteroarylene having 1 or 2 nitrogen ring atoms;
L^{1g} is a bond, —OH₂—, —NH—, or—O—; and

### L^{2g} is

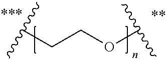

wherein n is 1, 2, 3, 4, or 5, and

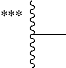

represents a covalent bond to L^{1g};
wherein

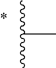

represents a covalent bond to the Y group of Formula (I), or when Y is a bond, a covalent bond to the quinazoline group of Formula (I), and

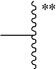

represents a covalent bond to the methylene group of Formula (I).

In another embodiment, L is a divalent linker of Formula (L-g-i):

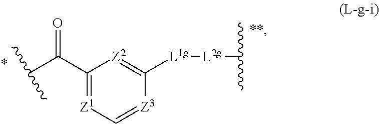

wherein:
L^{1g} is a bond, —CH₂—, —NH—, or —O—;

### L^{2g} is

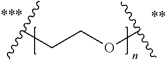

wherein n is 1, 2, 3, 4, or 5, and

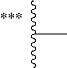

represents a covalent bond to L^{1g};
Z¹, Z², and Z³ are each independently selected from N or CH, provided that one or two of Z¹, Z², and Z³ is N;
wherein

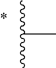

represents a covalent bond to the Y group of Formula (I), or when Y is a bond, a covalent bond to the quinazoline group of Formula (I), and

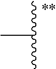

represents a covalent bond to the methylene group of Formula (I).

In another embodiment, L is a divalent linker of Formula (L-g) selected from the group consisting of:

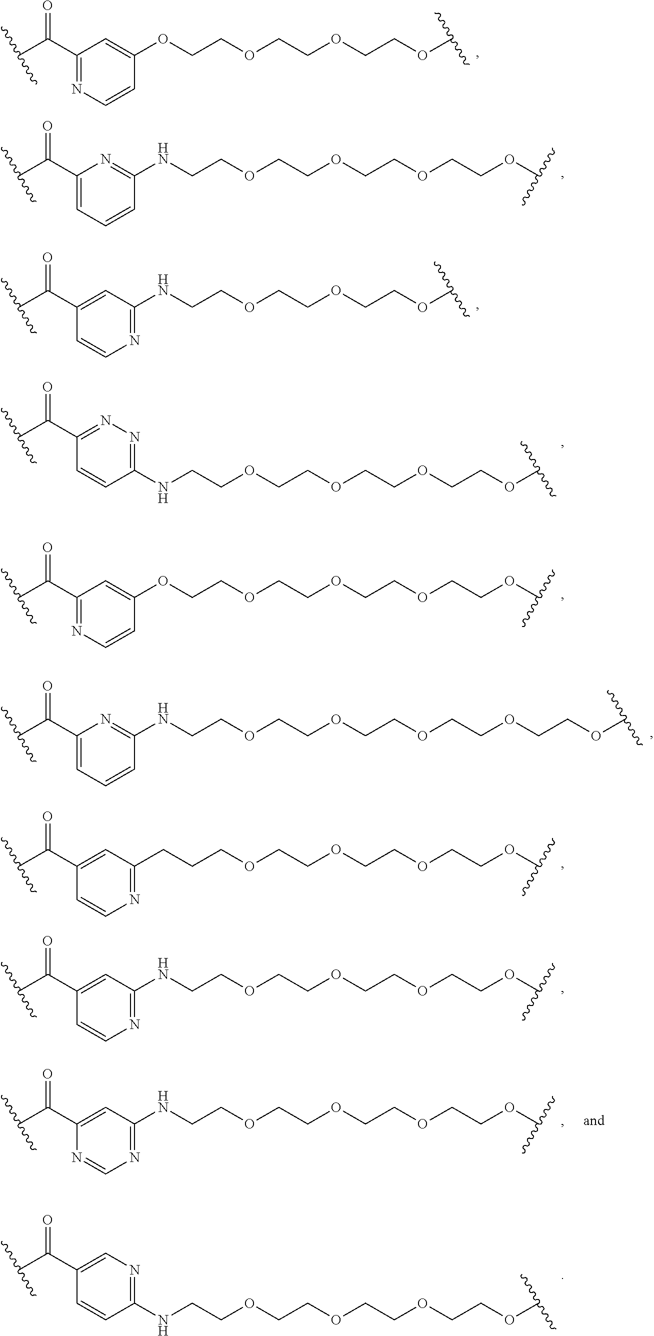

In another embodiment, L is a divalent linker of Formula (L-h):

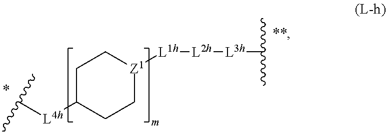

or a stereoisomer thereof,
wherein:
each Z¹ is independently N or CH;
L^{1h} is a bond, —C(O)—, —C(O)—NH—, or —NHC(O)—;
L^{2h} is C₂₋₁₀ linear alkylene or

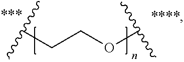

wherein n is 1, 2, 3, or 4, and

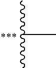

represents a covalent bond to L^{1h} and

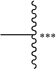

represents a covalent bond to L^{3h};
L^{3h} is a bond, —C(O)CH₂—, —O—(C₃₋₆ cycloalkylene)-O—, or —C(O)NH(CH₂)₃OCH₂—;
L^{4h} is a bond, —C(O)—, —CH₂C(O)—, or —C(O)CH₂—; and
m is 1, 2, or 3;
wherein

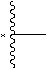

represents a covalent bond to the Y group of Formula (I), or when Y is a bond, a covalent bond to the quinazoline group of Formula (I), and

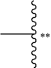

represents a covalent bond to the methylene group of Formula (I).

In another embodiment, L is a divalent linker of Formula (L-h) selected from the group consisting of:

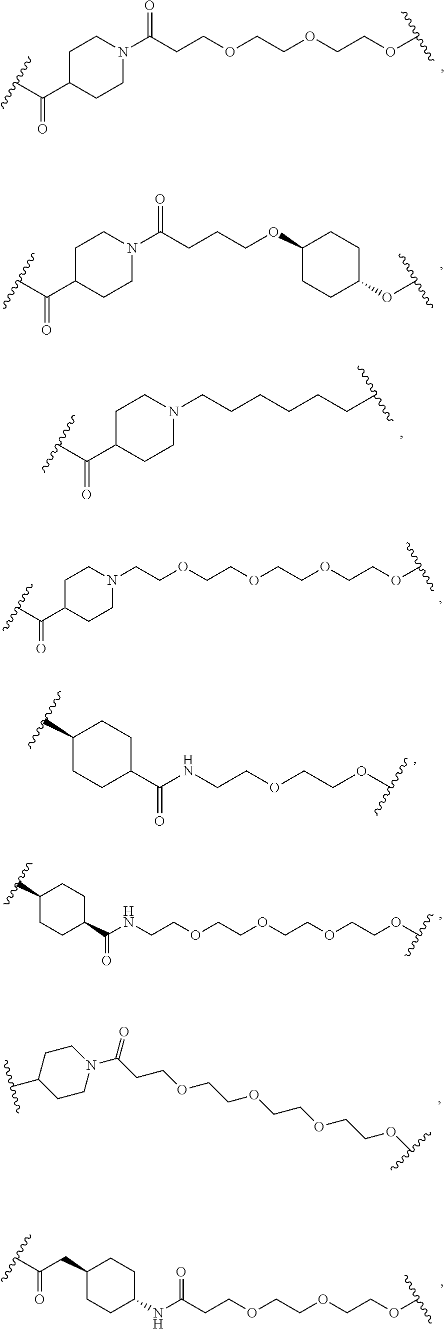

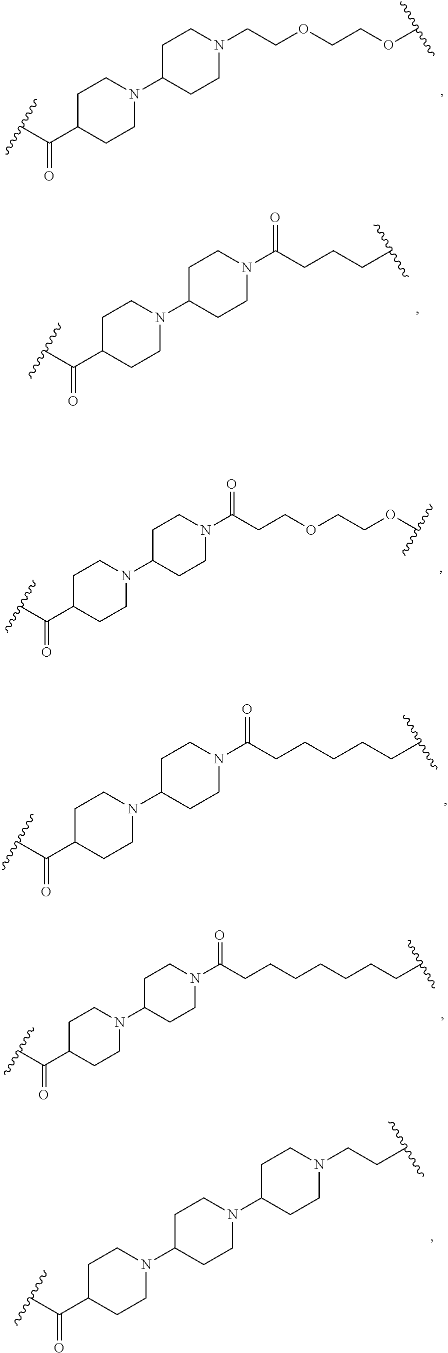 [SMILES: *OCCOCCN1CCC(N2CCC(C(*)=O)CC2)CC1 ; *CCCC(=O)N1CCC(N2CCC(C(*)=O)CC2)CC1 ; *OCCOCCC(=O)N1CCC(N2CCC(C(*)=O)CC2)CC1 ; *CCCCCC(=O)N1CCC(N2CCC(C(*)=O)CC2)CC1 ; *CCCCCCCC(=O)N1CCC(N2CCC(C(*)=O)CC2)CC1 ; *CCN1CCC(N2CCC(N3CCC(C(*)=O)CC3)CC2)CC1]

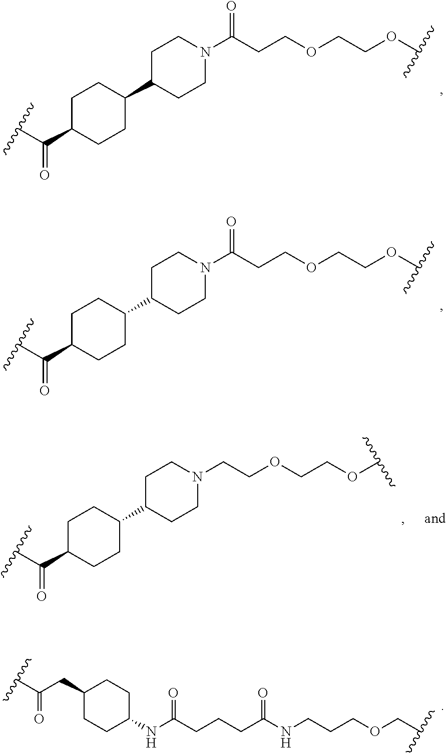 [SMILES: *OCCOCCC(=O)N1CCC([C@H]2CC[C@@H](C(*)=O)CC2)CC1 ; *OCCOCCC(=O)N1CCC([C@H]2CC[C@H](C(*)=O)CC2)CC1 ; *OCCOCCN1CCC([C@H]2CC[C@H](C(*)=O)CC2)CC1 ; *COCCCNC(=O)CCCC(=O)N[C@H]1CC[C@H](CC(*)=O)CC1]

In another embodiment, L is a divalent linker of Formula (L-i):

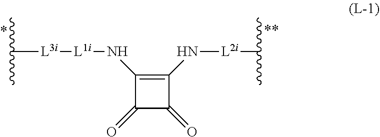

wherein:
L¹ⁱ is a bond, C₁₋₁₂ linear alkylene, or

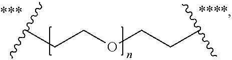

wherein n is 1, 2, 3, 4, or 5, and

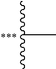

represents a covalent bond to L³ⁱ and

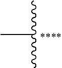

represents a covalent bond to NH;
L²ⁱ is a bond, C₁₋₁₂ linear alkylene, or

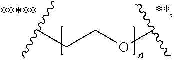

wherein n is 1, 2, 3, 4, or 5, and

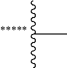

represents a covalent bond to HN; and
L³ⁱ is a bond or —C(O)—;
wherein

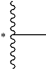

represents a covalent bond to the Y group of Formula (I), or when Y is a bond, a covalent bond to the quinazoline group of Formula (I), and

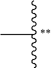

represents a covalent bond to the methylene group of Formula (I).

In another embodiment, L is a divalent linker of Formula (L-i) selected from the group consisting of:

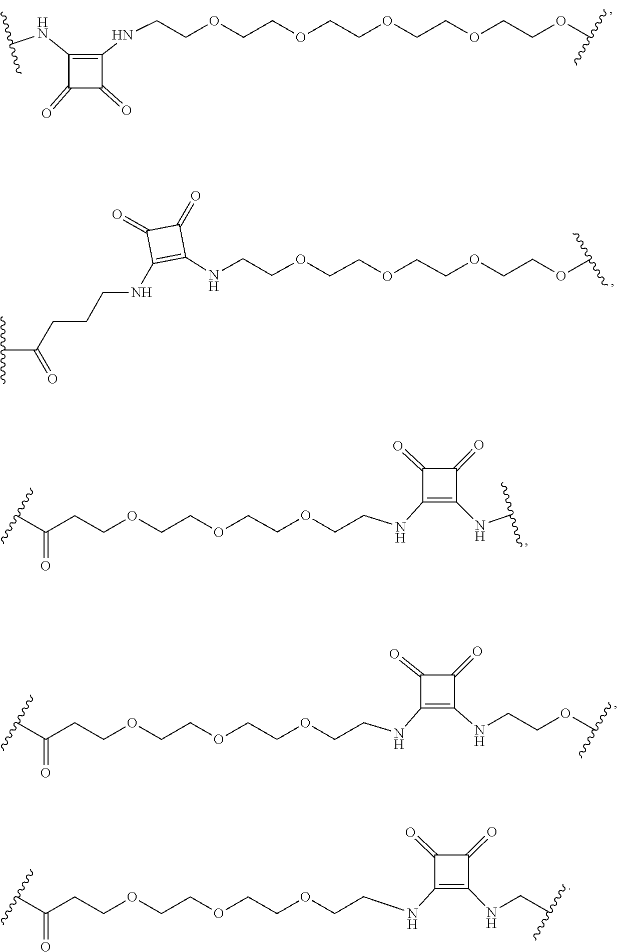 [SMILES: *Nc1c(NCCOCCOCCOCCOCCO*)c(=O)c1=O ; *OCCOCCOCCOCCNc1c(NCCCC(*)=O)c(=O)c1=O ; *Nc1c(NCCOCCOCCOCCC(*)=O)c(=O)c1=O ; *OCCNc1c(NCCOCCOCCOCCC(*)=O)c(=O)c1=O ; *CNc1c(NCCOCCOCCOCCC(*)=O)c(=O)c1=O]

In another embodiment, L is a divalent linker of Formula (L-j):

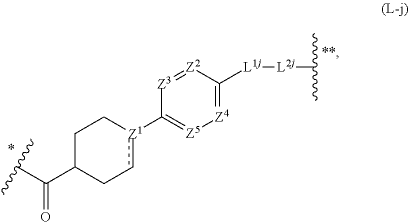

or a stereoisomer thereof,
wherein:

### Z¹ is C, CH, or N;

each of Z², Z³, Z⁴ and Z⁵ is independently CH or N, provided that no more than two of Z², Z³, Z⁴ and Z⁵ are N;

### L^{1j} is —NH—, —C(O)NH—, —NHC(O)—, or —O—;

L^{2j} is C₁₋₆ linear alkylene or

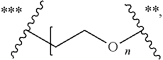

wherein n is 1 or 2, and

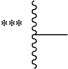

represents a covalent bond to L^{1j}; and [symbol]
represents a single bond or a double bond;
wherein

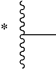

represents a covalent bond to the Y group of Formula (I), or when Y is a bond, a covalent bond to the quinazoline group of Formula (I), and

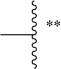

represents a covalent bond to the methylene group of Formula (I).

In another embodiment, L is a divalent linker of Formula (L-j) selected from the group consisting of:

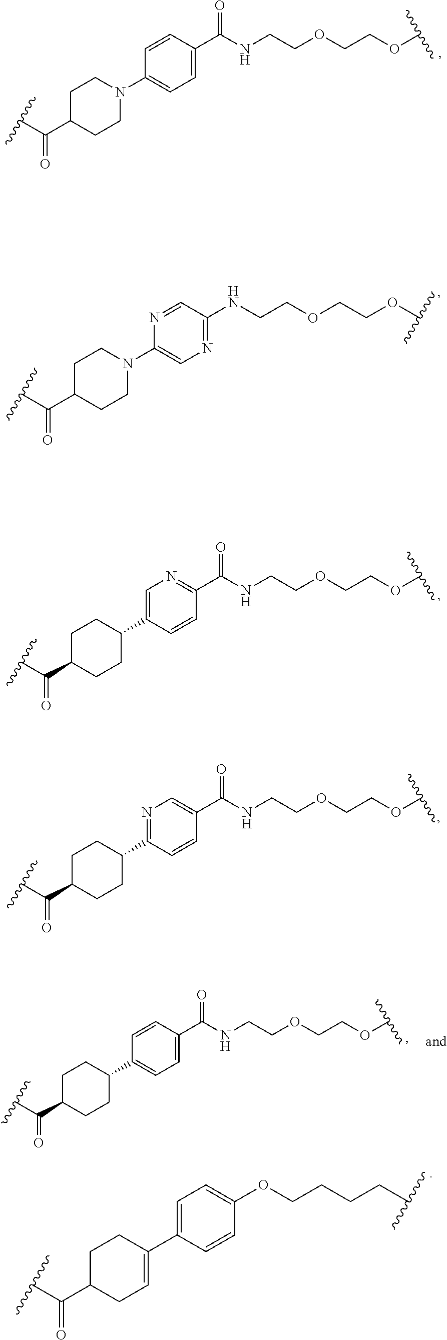 [SMILES: *OCCOCCNC(=O)c1ccc(N2CCC(C(*)=O)CC2)cc1 ; *OCCOCCNc1cnc(N2CCC(C(*)=O)CC2)cn1 ; *OCCOCCNC(=O)c1ccc([C@H]2CC[C@H](C(*)=O)CC2)cn1 ; *OCCOCCNC(=O)c1ccc([C@H]2CC[C@H](C(*)=O)CC2)nc1 ; *OCCOCCNC(=O)c1ccc([C@H]2CC[C@H](C(*)=O)CC2)cc1 ; *CCCCOc1ccc(C2=CCC(C(*)=O)CC2)cc1]

In another embodiment, L is a divalent linker of Formula (L-k):

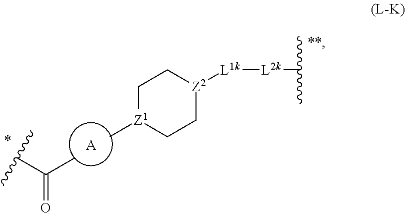

or a stereoisomer thereof,
wherein:
Ring A is phenyl or a 5 or 6 membered heteroarylene having 1 or 2 nitrogen ring atoms; each of Z¹ and Z² is independently CH or N;
L^{1k} is a bond, —C(O)—, —C(O)NH— or —NHC(O)—; and
L^{2k} is a C₃₋₈ straight chain alkylene or

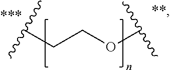

wherein n is 1, 2, or 3, and

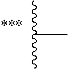

represents a covalent bond to L^{1k};
wherein

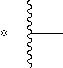

represents a covalent bond to the Y group of Formula (I), or when Y is a bond, a covalent bond to the quinazoline group of Formula (I), and

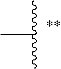

represents a covalent bond to the methylene group of Formula (I).

In another embodiment, L is a divalent linker of Formula (L-k) selected from the group consisting of:

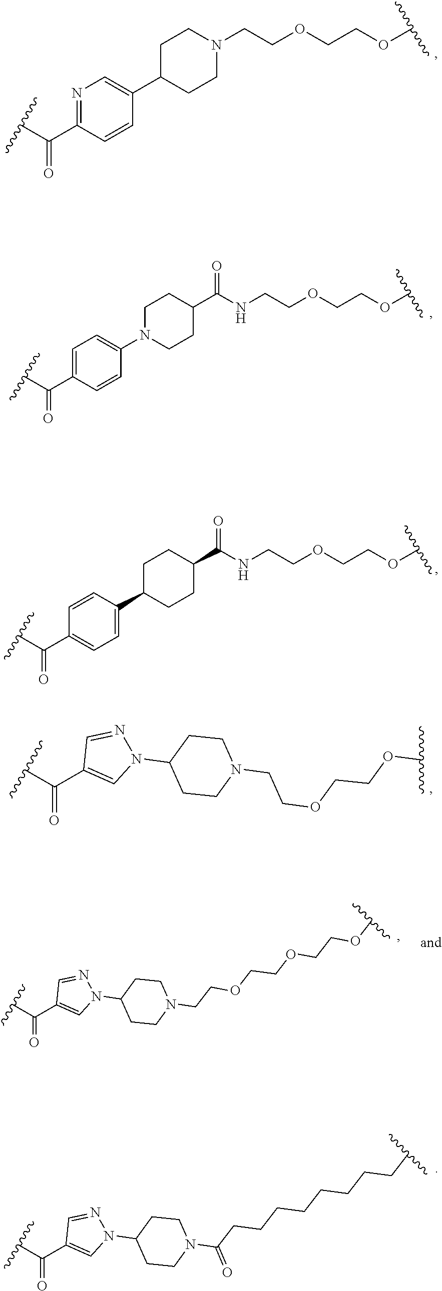 [SMILES: *OCCOCCN1CCC(c2ccc(C(*)=O)nc2)CC1 ; *OCCOCCNC(=O)C1CCN(c2ccc(C(*)=O)cc2)CC1 ; *OCCOCCNC(=O)[C@H]1CC[C@@H](c2ccc(C(*)=O)cc2)CC1 ; *OCCOCCN1CCC(n2cc(C(*)=O)cn2)CC1 ; *OCCOCCOCCN1CCC(n2cc(C(*)=O)cn2)CC1 ; *CCCCCCCCC(=O)N1CCC(n2cc(C(*)=O)cn2)CC1]

In another embodiment, L is a divalent linker of Formula (L-m):

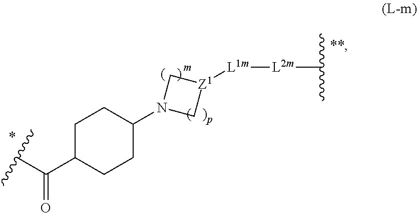

or a stereoisomer thereof,
wherein:

### Z¹ is CH or N;

m is 1 or 2;
p is 1 or 2;
0, 1, or 2 hydrogen atoms of

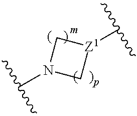

are replaced with F;
L^{1m} is a bond, —C(O)—, —C(O)NH—, —NHC(O)—, —S(O)₂NH— or —NHS(O)₂—; and
L^{2m} is C₃₋₆ linear alkylene, C₃₋₆ cycloalkylene, or

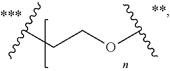

wherein n is 1 or 2, and

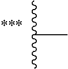

represents a covalent bond to L^{1m};
wherein

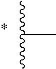

represents a covalent bond to the Y group of Formula (I), or when Y is a bond, a covalent bond to the quinazoline group of Formula (I), and

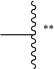

represents a covalent bond to the methylene group of Formula (I).

In another embodiment, L is a divalent linker of Formula (L-m) selected from the group consisting of:

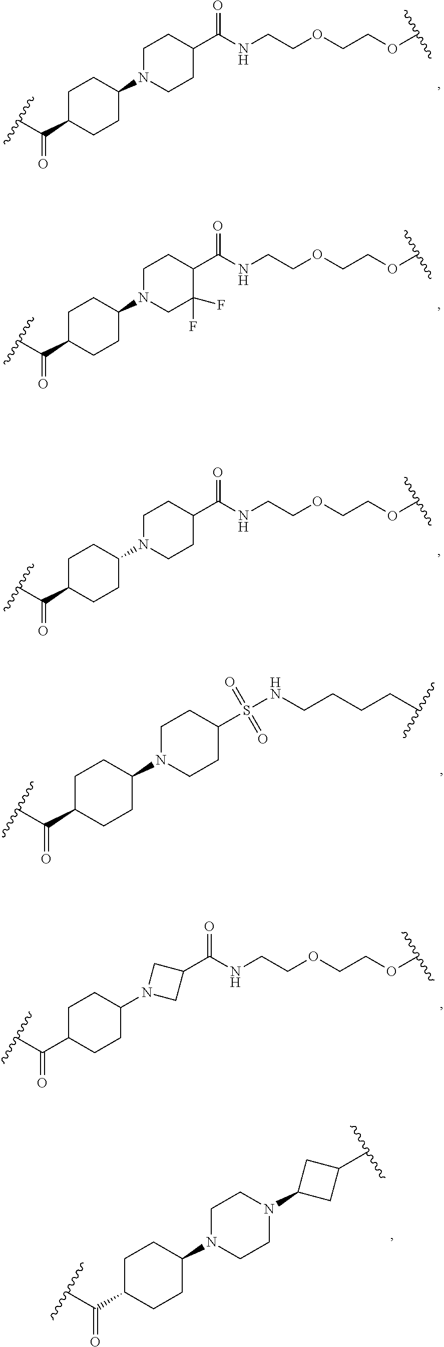

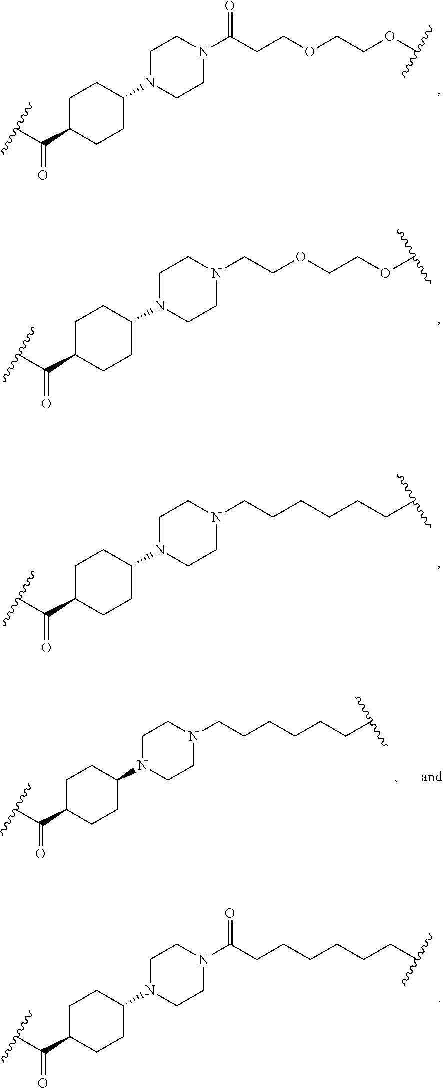 [SMILES: *OCCOCCC(=O)N1CCN([C@H]2CC[C@H](C(*)=O)CC2)CC1 ; *OCCOCCN1CCN([C@H]2CC[C@H](C(*)=O)CC2)CC1 ; *CCCCCCN1CCN([C@H]2CC[C@H](C(*)=O)CC2)CC1 ; *CCCCCCN1CCN([C@H]2CC[C@@H](C(*)=O)CC2)CC1 ; *CCCCCCC(=O)N1CCN([C@H]2CC[C@H](C(*)=O)CC2)CC1]

In another embodiment, L is a divalent linker of Formula (L-n-i):

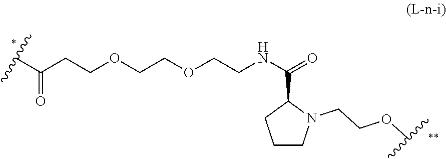 [SMILES: *OCCN1CCC[C@H]1C(=O)NCCOCCOCCC(*)=O]

wherein

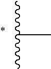

represents a covalent bond to the Y group of Formula (I), or when Y is a bond, a covalent bond to the quinazoline group of Formula (I), and

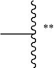

represents a covalent bond to the methylene group of Formula (I).

In another embodiment, L is a divalent linker of Formula (L-n-ii):

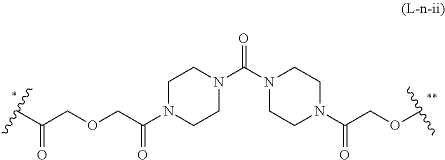 [SMILES: *OCC(=O)N1CCN(C(=O)N2CCN(C(=O)COCC(*)=O)CC2)CC1]

wherein

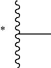

represents a covalent bond to the Y group of Formula (I), or when Y is a bond, a covalent bond to the quinazoline group of Formula (I), and

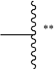

represents a covalent bond to the methylene group of Formula (I).

In another embodiment, L is a divalent linker of Formula (L-n-iii):

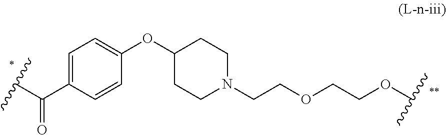 [SMILES: *OCCOCCN1CCC(Oc2ccc(C(*)=O)cc2)CC1]

wherein

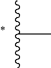

represents a covalent bond to the Y group of Formula (I), or when Y is a bond, a covalent bond to the quinazoline group of Formula (I), and

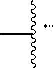

represents a covalent bond to the methylene group of Formula (I).

In another embodiment, L is a divalent linker of Formula (L-n-iv):

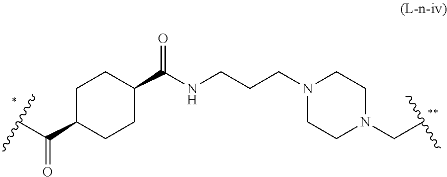 [SMILES: *CN1CCN(CCCNC(=O)[C@H]2CC[C@@H](C(*)=O)CC2)CC1]

wherein

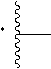

represents a covalent bond to the Y group of Formula (I), or when Y is a bond, a covalent bond to the quinazoline group of Formula (I), and

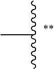

represents a covalent bond to the methylene group of Formula (I).

In one embodiment of the disclosure, Y is selected from a bond; —NH—; —(C₁₋₁₂ alkylene)-, wherein 1, 2, or 3 methylene units are replaced with —O—, —NH—, —C(O)—, —NHC(O)—, —C(O)NH—, —(C₃₋₆ cycloalkylene)-, —(C₃₋₆ cycloalkenylene)-, 3- to 6-membered heterocycloalkylene, arylene, or heteroarylene; or —(C₂₋₁₂ alkenylene)-, wherein 1, 2, or 3 methylene units are replaced with —O—, —NH—, —C(O)—, —NHC(O)—, —C(O)NH—, —(C₃₋₆ cycloalkylene)-, —(C₃₋₆ cycloalkenylene)-, 3- to 6-membered heterocycloalkylene, arylene, or heteroarylene.

In another embodiment, Y is selected from a bond; —NH—; —(C1-6 alkylene)-O—; —(C2-6 alkenylene)-O—; —(C1-6 alkylene)-C(O)—; —(C2-6 alkenylene)-C(O)—; phenylene; piperidinylene; —(C1-6 alkylene)-O-phenylene-; —(C2-6 alkenylene)-O-piperidinylene; —(C1-5 alkylene)-NH—, wherein 0, 1, or 2 methylene units are replaced with —O—; —NH—(C1-5 alkylene)-NH—; —(C3-6 cycloalkylene)-NH—; —(C3-6 cycloalkenylene)-NH—; or

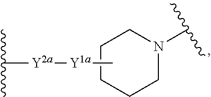

wherein Y1a is a bond, —O—, —NH—, —NHC(O)—, —C(O)NH—, or C1-3 alkylene; and Y2a is a bond, —O—, —NH—, —NHC(O)—, —C(O)NH—, or C1-3 alkylene. In another embodiment, Y is —NH—.

In another embodiment, Y is selected from the group consisting of:

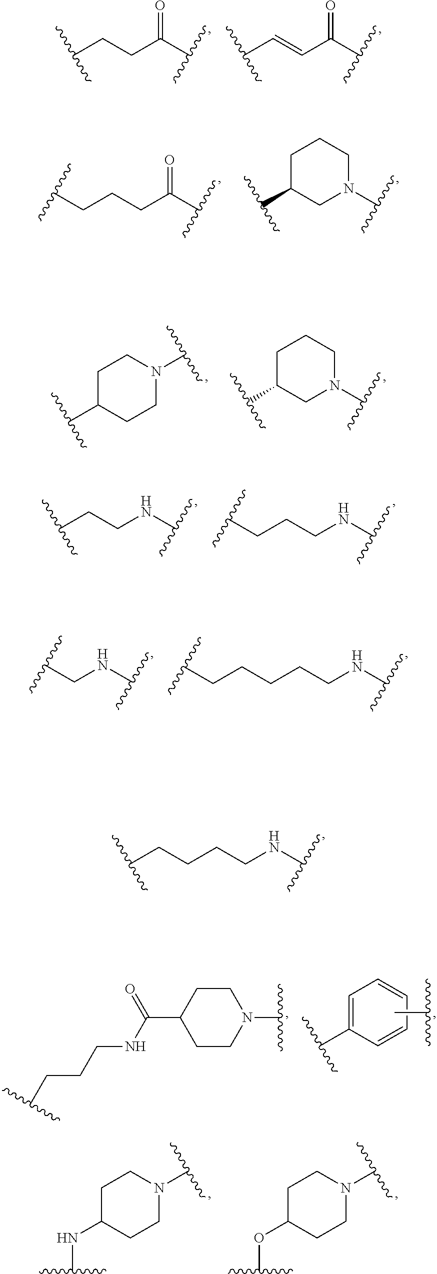

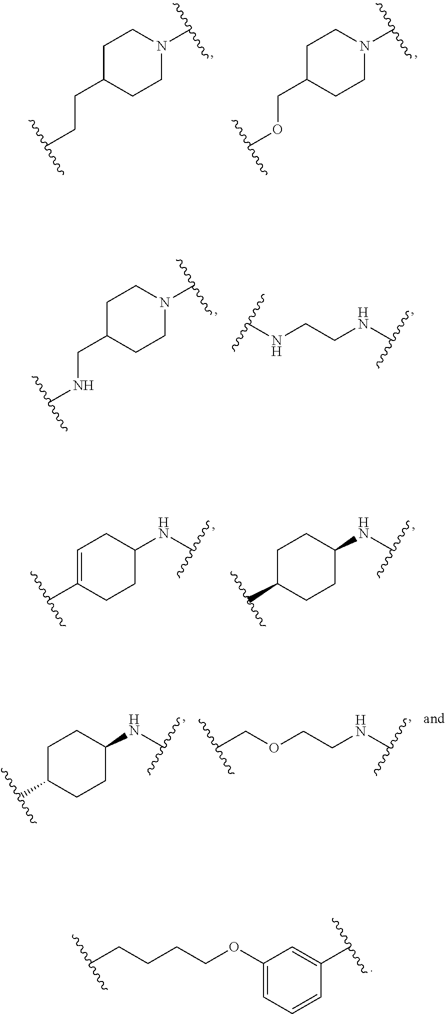

In another embodiment, R¹ is methyl, ethyl, 1-propyl, 2-propyl, 1-butyl, 2-butyl, or t-butyl. In another embodiment, R¹ is methyl. In another embodiment, R¹ is ethyl. In another embodiment, R¹ is cyclopropyl, cyclobutyl, cyclopentyl, or cyclohexyl.

In another embodiment, R² and R³ are each independently hydrogen, methyl, ethyl, 1-propyl, 2-propyl, 1-butyl, 2-butyl, or t-butyl. In another embodiment, R² is isopropyl and R³ is methyl. In another embodiment, R² is t-butyl and R³ is hydrogen.

In another embodiment, the compound of Formula (I) is selected from a compound as listed in Table 1:

TABLE 1

| Ex. | |
|---|---|
| No. | Structure |
| 1 | 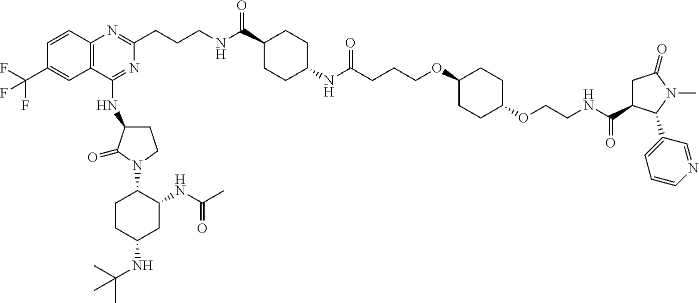 [SMILES: CC(=O)N[C@@H]1C[C@H](NC(C)(C)C)CC[C@@H]1N1CC[C@H](Nc2nc(CCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] |
| 2 | 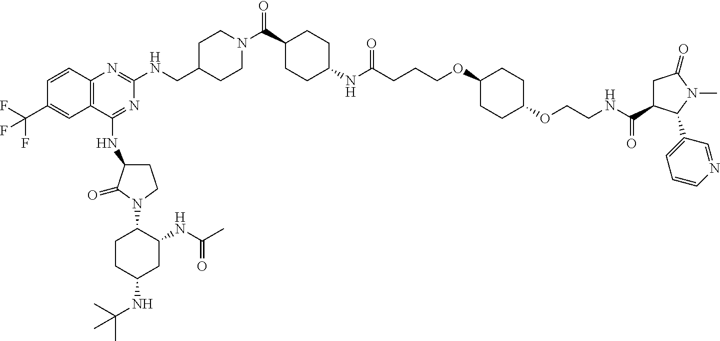 [SMILES: CC(=O)N[C@@H]1C[C@H](NC(C)(C)C)CC[C@@H]1N1CC[C@H](Nc2nc(NCC3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| 3 | 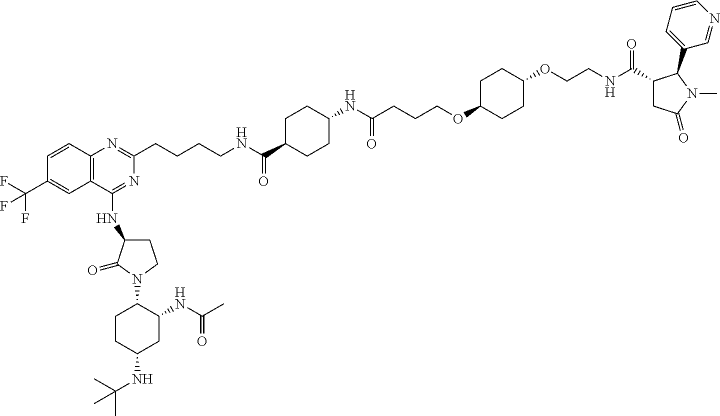 [SMILES: CC(=O)N[C@@H]1C[C@H](NC(C)(C)C)CC[C@@H]1N1CC[C@H](Nc2nc(CCCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] |
| 4 | 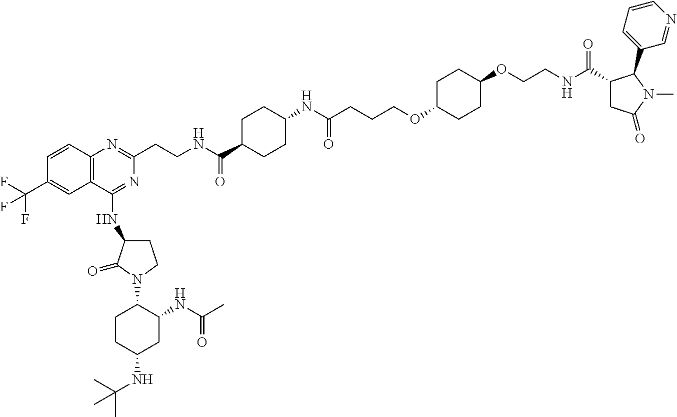 [SMILES: CC(=O)N[C@@H]1C[C@H](NC(C)(C)C)CC[C@@H]1N1CC[C@H](Nc2nc(CCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] |
| 5 | 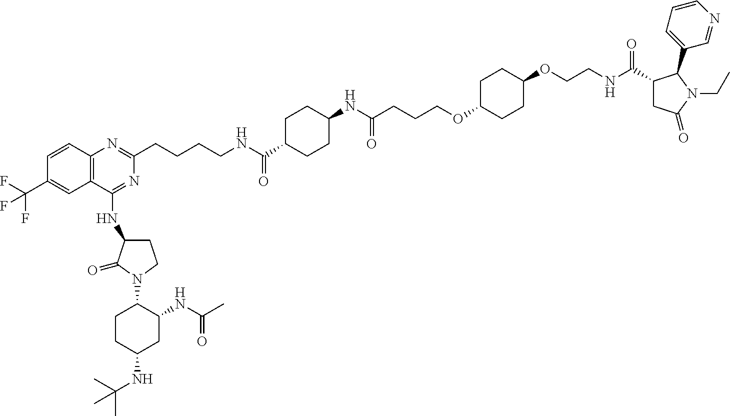 [SMILES: CCN1C(=O)C[C@H](C(=O)NCCO[C@H]2CC[C@H](OCCCC(=O)N[C@H]3CC[C@H](C(=O)NCCCCc4nc(N[C@H]5CCN([C@H]6CC[C@@H](NC(C)(C)C)C[C@H]6NC(C)=O)C5=O)c5cc(C(F)(F)F)ccc5n4)CC3)CC2)[C@H]1c1cccnc1] |
| 6 | 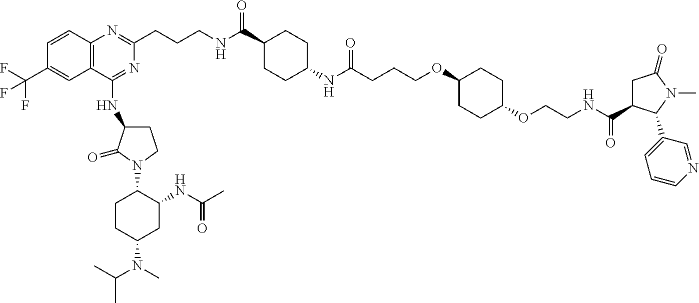 [SMILES: CC(=O)N[C@@H]1C[C@H](N(C)C(C)C)CC[C@@H]1N1CC[C@H](Nc2nc(CCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] |
| 7 | 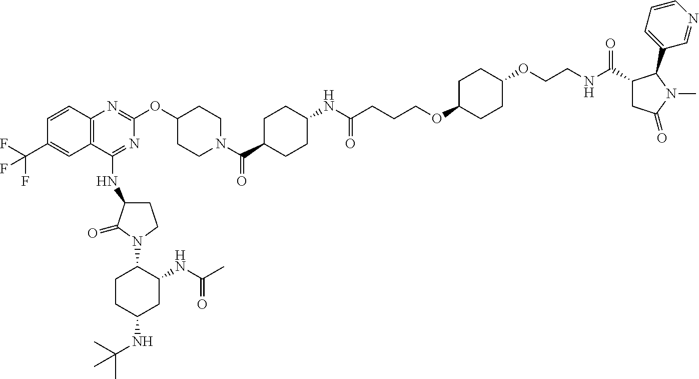 [SMILES: CC(=O)N[C@@H]1C[C@H](NC(C)(C)C)CC[C@@H]1N1CC[C@H](Nc2nc(OC3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| 8 | 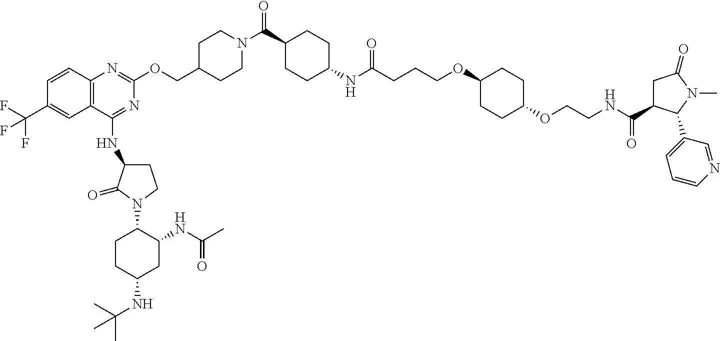 [SMILES: CC(=O)N[C@@H]1C[C@H](NC(C)(C)C)CC[C@@H]1N1CC[C@H](Nc2nc(OCC3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| 9 | 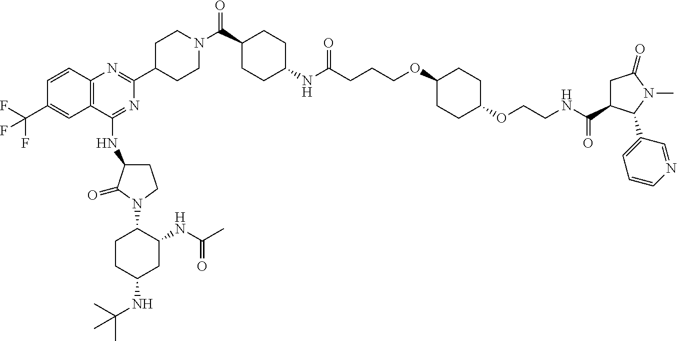 [SMILES: CC(=O)N[C@@H]1C[C@H](NC(C)(C)C)CC[C@@H]1N1CC[C@H](Nc2nc(C3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| 10 | 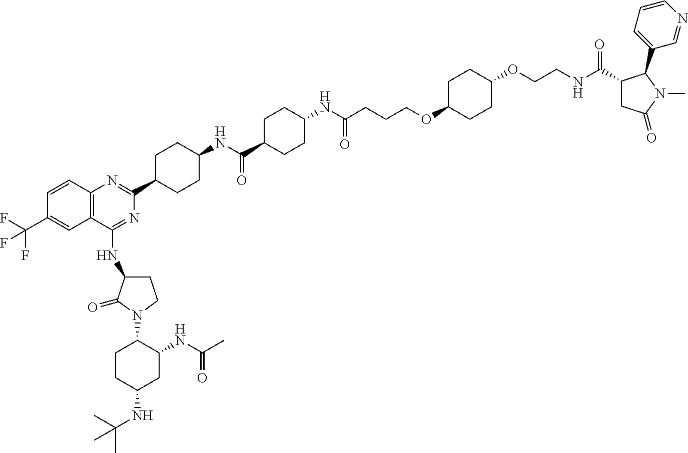 [SMILES: CC(=O)N[C@@H]1C[C@H](NC(C)(C)C)CC[C@@H]1N1CC[C@H](Nc2nc([C@H]3CC[C@@H](NC(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| 11 | 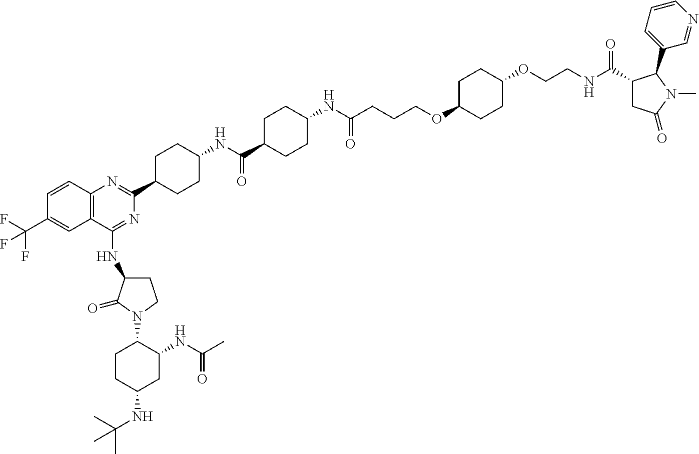 [SMILES: CC(=O)N[C@@H]1C[C@H](NC(C)(C)C)CC[C@@H]1N1CC[C@H](Nc2nc([C@H]3CC[C@H](NC(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| 12 | 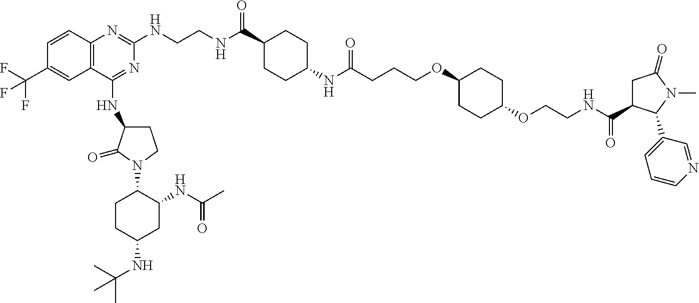 [SMILES: CC(=O)N[C@@H]1C[C@H](NC(C)(C)C)CC[C@@H]1N1CC[C@H](Nc2nc(NCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] |
| 13 | 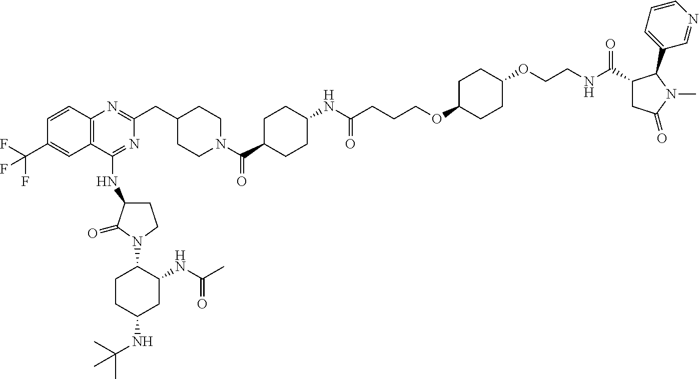 [SMILES: CC(=O)N[C@@H]1C[C@H](NC(C)(C)C)CC[C@@H]1N1CC[C@H](Nc2nc(CC3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| 14 | 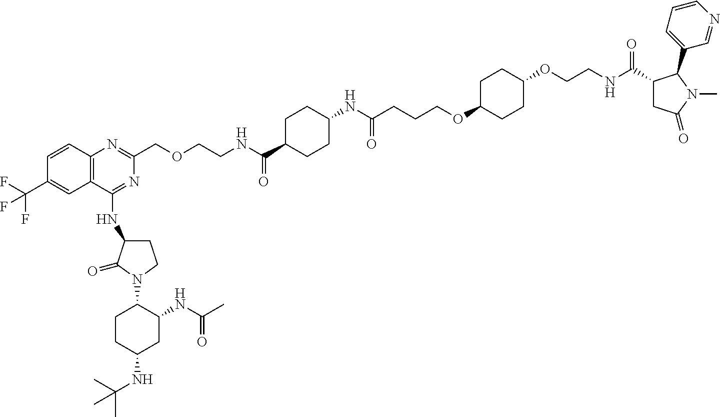 [SMILES: CC(=O)N[C@@H]1C[C@H](NC(C)(C)C)CC[C@@H]1N1CC[C@H](Nc2nc(COCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] |

### Definitions

As used herein and in the claims, the singular forms “a” and “the” include plural reference unless the context clearly dictates otherwise.

As used herein and in the claims, the term “comprising” encompasses “including” or “consisting” e.g. a composition “comprising” X may consist exclusively of X or may include something additional, e.g., X+Y.

The term “consisting essentially of” limits the scope of the feature to the specified materials or steps and those that do not materially affect the basic characteristic(s) of the claimed feature.

The term “consisting of” excludes the presence of any additional component(s).

The term “pathogenic cells” includes a cell subset that causes or is capable of causing disease. Examples of pathogenic cells include, but are not limited to, pathogenic immune cells, cancer or tumor cells, and stromal cells. A pathogenic cell can also be a pathogenic agent capable of causing an infection, such as a virus or a bacterial cell.

The term “pathogenic immune cells” includes a particular immune cell subset that causes or is capable of causing disease. These cellular subsets are resident cells or are recruited to particular locations and secrete cytokines, chemokines and other mediators and contribute to the persistence and progression of disease such as cancer in the case of a tumor microenvironment or chronic inflammation of the lung in the case of asthma. Examples of pathogenic immune cells include, but are not limited to myeloid-derived suppressor cells (MDSCs), T regulatory cells (Tregs), neutrophils, macrophages, B regulatory cells (Bregs), CD8 regulatory cells, (CD8regs), and exhausted T cells.

The term “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

The terms “effective amount” and “therapeutically effective amount” refer to an amount of a compound, or antibody, or antigen-binding portion thereof, according to the invention, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the invention which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the invention, and the age, body weight, general health, sex and diet of the patient. Such a therapeutically effective amount can be determined routinely by one of ordinary skill in the art having regard to their own knowledge, the state of the art, and this disclosure.

The term “alkyl” represents a saturated, linear or branched hydrocarbon moiety having the specified number of carbon atoms. The term “C₁₋₃ alkyl” refers to an unsubstituted alkyl moiety containing 1, 2 or 3 carbon atoms; exemplary alkyls include methyl, ethyl and propyl.

The term “alkylene” represents a saturated, linear or branched hydrocarbon moiety having the specified number of carbon atoms, with two points of attachment. The two points of attachment can be from the same or different carbon atoms. The term “C₁₋₃ alkylene” refers to an unsubstituted alkyl moiety containing 1, 2 or 3 carbon atoms with two points of attachment; exemplary C₁₋₃ alkylene groups include methylene, ethylene and propylene.

The term “alkenyl” represents an unsaturated, linear or branched hydrocarbon moiety having the specified number of carbon atoms. The term “C₂₋₆ alkenyl” refers to an unsubstituted alkenyl moiety containing 2, 3, 4, 5, or 6 carbon atoms; exemplary alkenyls include propenyl, butenyl, pentenyl and hexenyl.

The term “alkenylene” represents an unsaturated, linear or branched hydrocarbon moiety having the specified number of carbon atoms, with two points of attachment. The two points of attachment can be from the same or different carbon atoms. The term “C₂₋₆ alkenylene” refers to an unsubstituted alkenyl moiety containing 2, 3, 4, 5, or 6 carbon atoms with two points of attachment; exemplary C₂₋₆ alkenylene groups include propenylene, butenylene, pentenylene and hexenylene.

The term “cycloalkyl” represents a saturated cyclic hydrocarbon moiety having the specified number of carbon atoms. The term “C₃₋₆ cycloalkyl” refers to an unsubstituted cycloalkyl moiety containing 3, 4, 5 or 6 carbon atoms; exemplary cycloalkyls include cyclopropyl, cyclobutyl, cyclopentyl, and cyclohexyl.

The term “cycloalkylene” represents a saturated cyclic hydrocarbon moiety having the specified number of carbon atoms, with two points of attachment. The two points of attachment can be from the same or different carbon atoms. The term “C₄₋₆ cycloalkylene” refers to an unsubstituted cycloalkylene moiety containing 4, 5, or 6 carbon atoms with two points of attachment. Exemplary cycloalkylene groups include cyclobutane-1,3-diyl, cyclopentane-1,3-diyl, cyclohexane-1,3-diyl, or cyclohexane-1,4-diyl.

The term “cycloalkenylene” represents an unsaturated cyclic hydrocarbon moiety having the specified number of carbon atoms, with two points of attachment. The two points of attachment can be from the same or different carbon atoms. The term “C₃₋₆ cycloalkenylene” refers to an unsubstituted cycloalkenylene moiety containing 3, 4, 5, or 6 carbon atoms with two points of attachment.

The term “heterocycloalkylene” refers to a saturated cyclic hydrocarbon moiety containing 1 or 2 heteroatoms independently selected from oxygen, sulphur or nitrogen atoms, with two points of attachment. The two points of attachment can be from the same or different carbon atoms. The term “3- to 6-membered heterocycloalkylene” refers to a 3- to 6-membered saturated cyclic moiety containing 2, 3, 4 or 5 carbon atoms in addition to 1 or 2 oxygen, sulphur or nitrogen atoms, with two points of attachment. Suitably, the 3- to 6-membered heterocycloalkylene group contains 1 oxygen or nitrogen atom. Suitably such group contains 3 carbon atoms and 1 oxygen or nitrogen atom, such as azetidindiyl or oxetandiyl. Suitably such group contains 4 or 5 carbon atoms and 1 oxygen or nitrogen atom, such as tetrahydrofurandiyl, tetrahydropyrandiyl, pyrrolidindiyl or piperidindiyl.

The term “bridged bicyclic cycloalkylene” refers to a saturated bicyclic hydrocarbon moiety having at least one bridge, with two points of attachment. A “bridge” is an unbranched chain of atoms or an atom or a valence bond connecting two bridgeheads, where a “bridgehead” is any skeletal atom of the ring system which is bonded to three or more skeletal atoms (excluding hydrogen). The two points of attachment can be from the same or different carbon atoms. The term “C₇₋₉ bridged bicyclic cycloalkylene” refers to an unsubstituted bridged bicyclic cycloalkylene moiety containing 7, 8, or 9 carbon atoms with two points of attachment.

The term “arylene” refers to a monocyclic or bicyclic ring system wherein at least one ring in the system is aromatic, with two points of attachment. Exemplary arylene groups include phenylene, biphenylene, naphthylene, and anthracylene.

The term “heteroarylene” refers to a monocyclic or bicyclic ring system wherein at least one ring in the system is aromatic, and having, in addition to carbon atoms, from one to five heteroatoms independently selected from oxygen, sulphur or nitrogen atoms, with two points of attachment. The term “5- to 6-membered heteroarylene” refers to a 5- to 6-membered cyclic aromatic moiety containing 2, 3, 4 or 5 carbon atoms in addition to 1, 2, or 3 heteroatoms independently selected from oxygen, sulphur or nitrogen atoms, with two points of attachment.

The skilled artisan will appreciate that salts, including pharmaceutically acceptable salts, of the compounds according to Formula (I) may be prepared. Indeed, in certain embodiments of the invention, salts including pharmaceutically-acceptable salts of the compounds according to Formula (I) may be preferred over the respective free or unsalted compound. Accordingly, the invention is further directed to salts, including pharmaceutically-acceptable salts, of the compounds according to Formula (I). The invention is further directed to free or unsalted compounds of Formula (I).

The salts, including pharmaceutically acceptable salts, of the compounds of the invention are readily prepared by those of skill in the art.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, trifluoroacetate, undecanoate, undecylenate, and valerate.

Representative pharmaceutically acceptable base addition salts include, but are not limited to, aluminium, 2-amino-2-(hydroxymethyl)-1,3-propanediol (TRIS, tromethamine), arginine, benethamine (N-benzylphenethylamine), benzathine (N,N′-dibenzylethylenediamine), b/s-(2-hydroxyethyl)amine, bismuth, calcium, chloroprocaine, choline, clemizole (1-p chlorobenzyl-2-pyrrolidine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

The compounds according to Formula (I) may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may be present in a substituent such as an alkyl group. Where the stereochemistry of a chiral center present in a compound of Formula (I), or in any chemical structure illustrated herein, if not specified the structure is intended to encompass all individual stereoisomers and all mixtures thereof. Thus, compounds according to Formula (I) containing one or more chiral centers may be used as racemic mixtures, enantiomerically enriched mixtures, or as enantiomerically pure individual stereoisomers.

A mixture of stereoisomers in which the relative configuration of all of the stereocenters is known may be depicted using the symbol “&” together with an index number (e.g., “&1”). For example, a group of two stereogenic centers labeled with the symbol “&1” represents a mixture of two possible stereoisomers in which the two stereogenic centers have a relative configuration as depicted.

Divalent groups are groups having two points of attachment. For all divalent groups, unless otherwise specified, the orientation of the group is implied by the direction in which the formula or structure of the group is written.

Unless defined otherwise, all technical and scientific terms used herein have the same meaning as commonly understood by one of ordinary skill in the art to which this invention belongs. Although any compositions and methods similar or equivalent to those described herein can be used in the practice or testing of the methods of the disclosure, exemplary compositions and methods are described herein. Any of the aspects and embodiments of the disclosure described herein may also be combined. For example, the subject matter of any dependent or independent claim disclosed herein may be multiply combined (e.g., one or more recitations from each dependent claim may be combined into a single claim based on the independent claim on which they depend).

Ranges provided herein include all values within a particular range described and values about an endpoint for a particular range.

Concentrations described herein are determined at ambient temperature and pressure. This may be, for example, the temperature and pressure at room temperature or in a particular portion of a process stream. Preferably, concentrations are determined at a standard state of 25° C. and 1 bar of pressure.

### CCR2 Target and CCR2-Binding Moieties

The compounds of Formula (I) as disclosed herein are heterobifunctional synthetic agents designed such that one terminus interacts with a cell surface CCR2 target, while the other terminus binds a specific antibody. More specifically, the ARM simultaneously binds the cell surface CCR2 target as well as the specific antibody. This ternary complex directs immune surveillance to CCR2-expressing tissue/cells and unites the mechanisms of antibody function with the dose-control of small molecules. This mechanism may include antibody dependent cellular cytotoxicity (ADCC), antibody dependent cellular phagocytosis (ADCP), or complement dependant cytotoxicity (CDC), and preferably includes ADCC. The same Fc receptor expressing immune cells that initiate destruction of the ARM/antibody tagged cells also participate in presentation of endogenous antigens for the potential for long term cellular immunity.

The compounds of Formula (I) as disclosed herein include a CCR2-binding moiety that is capable of binding CCR2 present on the surface of a cell. In one embodiment, the CCR2 is expressed on a pathogenic cell.

In a further embodiment, the pathogenic cell is a pathogenic immune cell, a tumor cell or cancer cell, or a stromal cell (including stromal cells present in a tumor microenvironment).

In a further embodiment, the CCR2 target is present on the surface of a pathogenic agent selected from a virus or a bacterial cell. Examples of a virus expressing cell surface targets include, but are not limited to, influenza.

In a further embodiment, the pathogenic immune cells are monocytes, myeloid derived suppressor cells (MDSC), such as monocytic MDSCs (mMDSCs) and polymorphonuclear MDSCs (PMN_MDSCs), T regulatory cells (Tregs), neutrophils (e.g., N2 neutrophils), macrophages (e.g., M2 macrophages), B regulatory cells (Bregs, memory B cells), plasma cells, CD8 cells (e.g., CD8 regulatory cells (CD8regs), memory CD8 cells, effector CD8 cells, naïve CD8 Tcells, TEMRA), exhausted T cells, eosinophils, basophils, mast cells, dendritic cells, natural killer (NK cells), innate lymphoid cells, NK T cells (NKT), or γδT cells.

In a further embodiment, the pathogenic immune cells are myeloid derived suppressor cells (MDSC), such as monocytic MDSCs (mMDSCs) and polymorphonuclear MDSCs (PMN_MDSCs), T regulatory cells (Tregs), neutrophils (e.g., N2 neutrophils), macrophages (e.g., M2 macrophages), B regulatory cells (Bregs), CD8 regulatory cells (CD8regs), exhausted T cells.

In a further embodiment, the pathogenic immune cells expressing CCR2 are myeloid derived suppressor cells (MDSCs). In a further embodiment, the pathogenic immune cells expressing CCR2 are selected from monocytic MDSCs (mMDSCs) and polymorphonuclear MDSCs (PMN_MDSCs).

In a further embodiment, the tumor cells or cancer cells are solid tumor cells.

In a further embodiment, the tumor cells or cancer cells are lung cancer cells (e.g., non-small cell lung cancer (NSCLC) cells), hepatocellular carcinoma (HCC) cells, colorectal cancer (CRC) cells, cervical cancer cells (e.g., cervical squamous cell carcinoma (CESC) cells), head and neck cancer cells (e.g., head and neck squamous cell carcinoma (HNSC) cells), pancreatic cancer cells, prostate cancer cells (e.g., metastatic castration-resistant prostate cancer (mCRPC) cells), ovarian cancer cells, endometrial cancer cells, bladder cancer cells, or breast cancer cells, preferably NSCLC cells, HCC cells, or CRC cells.

In a further embodiment, the stromal cells are cancer associated fibroblasts (CAFs).

The present disclosure also provides a pharmaceutical composition comprising a compound of Formula (I) as disclosed herein, and a pharmaceutically acceptable excipient, carrier, or diluent.

### Anti-Cotinine Antibodies

The present disclosure provides an antibody, or antigen-binding fragment thereof, that binds to a cotinine moiety. As used herein, the term “anti-cotinine antibody or antigen-binding fragment thereof” refers to an antibody, or antigen binding fragment thereof that binds to a cotinine moiety. Cotinine has the following structure:

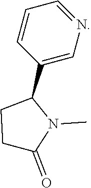

As used herein, the term “cotinine moiety” refers to cotinine or an analog of cotinine. Compounds of Formula (I) described herein comprise a cotinine moiety linked via a linker to a CCR2-binding moiety. In one embodiment, the cotinine moiety has the following structure:

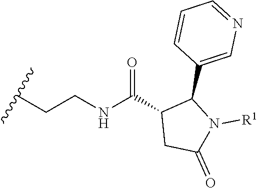

wherein R¹ is C₁₋₄ alkyl or C₃₋₆ cycloalkyl. In another embodiment, R¹ is methyl, ethyl, 1-propyl, 2-propyl, 1-butyl, 2-butyl, or t-butyl. In another embodiment, R¹ is methyl. In another embodiment, R¹ is ethyl. In another embodiment, R¹ is cyclopropyl, cyclobutyl, cyclopentyl, or cyclohexyl.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanised, multispecific antibodies, including bispecific antibodies, and heteroconjugate antibodies; a single variable domain (e.g., a domain antibody (DAB)), antigen binding antibody fragments, Fab, F(ab′)2, Fv, disulphide linked Fv, single chain Fv, disulphide-linked scFv, diabodies, TANDABS, etc. and modified versions of any of the foregoing (for a summary of alternative “antibody” formats see Holliger and Hudson, Nature Biotechnology, 2005, 23(9): 1126-1136).

The term, full, whole or intact antibody, used interchangeably herein, refers to a heterotetrameric glycoprotein with an approximate molecular weight of 150,000 daltons. An intact antibody is composed of two identical heavy chains (HCs) and two identical light chains (LCs) linked by covalent disulphide bonds. This H2L2 structure folds to form three functional domains comprising two antigen-binding fragments, known as ‘Fab’ fragments, and a ‘Fc’ crystallisable fragment. The Fab fragment is composed of the variable domain at the amino-terminus, variable heavy (VH) or variable light (VL), and the constant domain at the carboxyl terminus, CH1 (heavy) and CL (light). The Fc fragment is composed of two domains formed by dimerization of paired CH2 and CH3 regions. The Fc may elicit effector functions by binding to receptors on immune cells or by binding C1q, the first component of the classical complement pathway. The five classes of antibodies IgM, IgA, IgG, IgE and IgD are defined by distinct heavy chain amino acid sequences, which are called μ, α, γ, ε and δ respectively, each heavy chain can pair with either a K or λ light chain. The majority of antibodies in the serum belong to the IgG class, there are four isotypes of human IgG (IgG1, IgG2, IgG3 and IgG4), the sequences of which differ mainly in their hinge region.

“CDRs” are defined as the complementarity determining region amino acid sequences of an antibody or antigen binding fragment thereof. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and variable domain regions within full-length antigen binding sequences, e.g. within an antibody heavy chain sequence or antibody light chain sequence, are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 4th Ed., U.S. Department of Health and Human Services, National Institutes of Health (1987).

It will be apparent to those skilled in the art that there are alternative numbering conventions for amino acid residues in variable domain sequences and full-length antibody sequences. There are also alternative numbering conventions for CDR sequences, for example those set out in Chothia et al., Nature, 1989, 342: 877-883. The structure and protein folding of the antigen binding protein may mean that other residues are considered part of the CDR sequence and would be understood to be so by a skilled person.

Other numbering conventions for CDR sequences available to a skilled person include “AbM” (University of Bath) and “contact” (University College London) methods.

Table 2 below represents one definition using each numbering convention for each CDR or binding unit. It should be noted that some of the CDR definitions may vary depending on the individual publication used.

TABLE 2

| | Kabat CDR | Chothia CDR | AbM CDR | Contact CDR |
|---|---|---|---|---|
| H1 | 31-35/35A/35B | 26-32/33/34 | 26-35/35A/35B | 30-35/35A/35B |
| H2 | 50-65 | 52-56 | 50-58 | 47-58 |
| H3 | 95-102 | 95-102 | 95-102 | 93-101 |
| L1 | 24-34 | 24-34 | 24-34 | 30-36 |
| L2 | 50-56 | 50-56 | 50-56 | 46-55 |
| L3 | 89-97 | 89-97 | 89-97 | 89-96 |

In a further embodiment, the anti-cotinine antibody is humanized. In a further embodiment, the Fc region of the anti-cotinine antibody is modified to increase ADCC activity, ADCP activity, and/or CDC activity, suitable modifications of which are provided below. In a further embodiment, the Fc region of the anti-cotinine antibody is modified to increase ADCC activity.

Fc engineering methods can be applied to modify the functional or pharmacokinetics properties of an antibody. Effector function may be altered by making mutations in the Fc region that increase or decrease binding to C1q or Fcγ receptors and modify CDC or ADCC activity respectively. Modifications to the glycosylation pattern of an antibody can also be made to change the effector function. The in vivo half-life of an antibody can be altered by making mutations that affect binding of the Fc to the FcRn (neonatal Fc receptor).

The term “effector function” as used herein refers to one or more of antibody-mediated effects including antibody-dependent cell-mediated cytotoxicity (ADCC), antibody-mediated complement activation including complement-dependent cytotoxicity (CDC), complement-dependent cell-mediated phagocytosis (CDCP), antibody dependent complement-mediated cell lysis (ADCML), and Fc-mediated phagocytosis or antibody-dependent cellular phagocytosis (ADCP).

The interaction between the Fc region of an antigen binding protein or antibody and various Fc receptors (FcR), including FcγRI (CD64), FcγRII (CD32), FcγRIII (CD16), FcRn, C1q, and type II Fc receptors is believed to mediate the effector functions of the antigen binding protein or antibody. Significant biological effects can be a consequence of effector functionality. Usually, the ability to mediate effector function requires binding of the antigen binding protein or antibody to an antigen and not all antigen binding proteins or antibodies will mediate every effector function.

Effector function can be assessed in a number of ways including, for example, evaluating ADCC effector function of antibody coated to target cells mediated by Natural Killer (NK) cells via FcγRIII, or monocytes/macrophages via FcγRI, or evaluating CDC effector function of antibody coated to target cells mediated by complement cascade via C1q. For example, an antibody, or antigen binding fragment thereof, of the present invention can be assessed for ADCC effector function in a Natural Killer cell assay. Examples of such assays can be found in Shields et al., The Journal of Biological Chemistry, 2001, 276: 6591-6604; Chappel et al., The Journal of Biological Chemistry, 1993, 268: 25124-25131; Lazar et al., PNAS, 2006, 103: 4005-4010.

Examples of assays to determine CDC function include those described in J Imm Meth, 1995, 184: 29-38.

The effects of mutations on effector functions (e.g., FcRn binding, FcγRs and C1q binding, CDC, ADCML, ADCC, ADCP) can be assessed, e.g., as described in Grevys et al., J Immunol., 2015,194(11): 5497-5508; Tam et al., Antibodies, 2017, 6(3): 12; or Monnet et al., mAbs, 2014, 6(2): 422-436.

Throughout this specification, amino acid residues in Fc regions, in antibody sequences or full-length antigen binding protein sequences, are numbered according to the EU index numbering convention.

Human IgG1 constant regions containing specific mutations have been shown to enhance binding to Fc receptors. In some cases these mutations have also been shown to enhance effector functions, such as ADCC and CDC, as described below. Antibodies, or antigen binding fragments thereof, of the present invention may include any of the following mutations.

Enhanced CDC: Fc engineering can be used to enhance complement-based effector function. For example (with reference to IgG1), K326W/E333S; S267E/H268F/S324T; and IgG1/IgG3 cross subclass can increase C1q binding; E345R (Diebolder et al., Science, 2014, 343: 1260-1293) and E345R/E430G/S440Y results in preformed IgG hexamers (Wang et al., Protein Cell, 2018, 9(1): 63-73).

Enhanced ADCC: Fc engineering can be used to enhance ADCC. For example (with reference to IgG1), F243L/R292P/Y300L/V3051/P396L; S239D/I332E; and S298A/E333A/K334A increase FcγRIIIa binding; S239D/I332E/A330L increases FcγRIIIa binding and decreases FcγRIIb binding; G236A/S239D/I332E improves binding to FcγRIIa, improves the FcγRIIa/FcγRIIb binding ratio (activating/inhibitory ratio), and enhances phagocytosis of antibody-coated target cells by macrophages. An asymmetric Fc in which one heavy chain contains L234Y/L235Q/G236W/S239M/H268D/D270E/S298A mutations and D270E/K326D/A330M/K334E in the opposing heavy chain, increases affinity for FcγRIIIa F158 (a lower-affinity allele) and FcγRIIIa V158 (a higher-affinity allele) with no increased binding affinity to inhibitory FcγRIIb (Mimoto et al., mAbs, 2013, 5(2): 229-236).

Enhanced ADCP: Fc engineering can be used to enhance ADCP. For example (with reference to IgG1), G236A/S239D/I332E increases FcγRIIa binding and increases FcγRIIIa binding (Richards, J. et al., Mol. Cancer Ther., 2008, 7: 2517-2527).

Increased co-engagement: Fc engineering can be used to increase co-engagement with FcRs. For example (with reference to IgG1), S267E/L328F increases FcγRIIb binding; N325S/L328F increases FcγRIIa binding and decreases FcγRIIIa binding Wang et al., Protein Cell, 2018, 9(1): 63-73).

In a further embodiment, an antibody, or antigen binding fragment thereof, of the present invention may comprise a heavy chain constant region with an altered glycosylation profile, such that the antibody, or antigen binding fragment thereof, has an enhanced effector function, e.g., enhanced ADCC, enhanced CDC, or both enhanced ADCC and CDC. Examples of suitable methodologies to produce an antibody, or antigen binding fragment thereof, with an altered glycosylation profile are described in WO 2003/011878, WO 2006/014679 and EP1229125.

The absence of the α1,6 innermost fucose residues on the Fc glycan moiety on N297 of IgG1 antibodies enhances affinity for FcγRIIIA. As such, afucosylated or low fucosylated monoclonal antibodies may have increased therapeutic efficacy (Shields et al., J Biol Chem., 2002, 277(30): 26733-40 and Monnet et al., mAbs, 2014, 6(2): 422-436).

In one embodiment there is provided an antibody, or antigen binding fragment thereof, comprising a chimeric heavy chain constant region. In an embodiment, the antibody, or antigen binding fragment thereof, comprises an IgG1/IgG3 chimeric heavy chain constant region, such that the antibody, or antigen binding fragment thereof, has an enhanced effector function, for example enhanced ADCC or enhanced CDC, or enhanced ADCC and CDC functions. For example, a chimeric antibody, or antigen binding fragment thereof, of the invention may comprise at least one CH2 domain from IgG3. In one such embodiment, the antibody, or antigen binding fragment thereof, comprises one CH2 domain from IgG3 or both CH2 domains may be from IgG3. In a further embodiment, the chimeric antibody, or antigen binding fragment thereof, comprises an IgG1 CH1 domain, an IgG3 CH2 domain, and an IgG3 CH3 domain. In a further embodiment, the chimeric antibody, or antigen binding fragment thereof, comprises an IgG1 CH1 domain, an IgG3 CH2 domain, and an IgG3 CH3 domain except for position 435 that is histidine.

In a further embodiment, the chimeric antibody, or antigen binding fragment thereof, comprises an IgG1 CH1 domain and at least one CH2 domain from IgG3. In an embodiment, the chimeric antibody, or antigen binding fragment thereof, comprises an IgG1 CH1 domain and the following residues, which correspond to IgG3 residues, in a CH2 domain: 274Q, 276K, 296F, 300F and 339T. In an embodiment, the chimeric antibody, or antigen binding fragment thereof, also comprises 356E, which corresponds to an IgG3 residue, within a CH3 domain. In an embodiment, the antibody, or antigen binding fragment thereof, also comprises one or more of the following residues, which correspond to IgG3 residues within a CH3 domain: 358M, 384S, 392N, 397M, 422I, 435R, and 436F.

Also provided is a method of producing an antibody, or antigen binding fragment thereof, according to the invention comprising the steps of:
a) culturing a recombinant host cell comprising an expression vector comprising a nucleic acid sequence encoding a chimeric Fc region having both IgG1 and IgG3 Fc region amino acid residues (e.g. as described above); and
b) recovering the antibody, or antigen binding fragment thereof.

Such methods for the production of antibody, or antigen binding fragment thereof, with chimeric heavy chain constant regions can be performed, for example, using the COMPLEGENT technology system available from BioWa, Inc. (Princeton, NJ) and Kyowa Hakko Kirin Co., Ltd. The COMPLEGENT system comprises a recombinant host cell comprising an expression vector in which a nucleic acid sequence encoding a chimeric Fc region having both IgG1 and IgG3 Fc region amino acid residues is expressed to produce an antibody, or antigen binding fragment thereof, having enhanced CDC activity, i.e. CDC activity is increased relative to an otherwise identical antibody, or antigen binding fragment thereof, lacking such a chimeric Fc region, as described in WO 2007/011041 and US 2007/0148165, each of which are incorporated herein by reference. In an alternative embodiment, CDC activity may be increased by introducing sequence specific mutations into the Fc region of an IgG chain. Those of ordinary skill in the art will also recognize other appropriate systems.

The present invention also provides a method of producing an antibody, or antigen binding fragment thereof, according to the invention comprising the steps of:
a) culturing a recombinant host cell comprising an expression vector comprising a nucleic acid encoding the antibody, or antigen binding fragment thereof, optionally wherein the FUT8 gene encoding alpha-1,6-fucosyltransferase has been inactivated in the recombinant host cell; and
b) recovering the antibody, or antigen binding fragment thereof.

Such methods for the production of an antibody, or antigen binding fragment thereof, can be performed, for example, using the POTELLIGENT technology system available from BioWa, Inc. (Princeton, NJ) in which CHOK1SV cells lacking a functional copy of the FUT8 gene produce monoclonal antibodies having enhanced ADCC activity that is increased relative to an identical monoclonal antibody produced in a cell with a functional FUT8 gene as described in U.S. Pat. Nos. 7,214,775, 6,946,292, WO 00/61739 and WO 02/31240, all of which are incorporated herein by reference. Those of ordinary skill in the art will also recognize other appropriate systems.

In one embodiment, the antibody, or antigen binding fragment thereof, is produced in a host cell in which the FUT8 gene has been inactivated. In a further embodiment, the antibody, or antigen binding fragment thereof, is produced in a −/−FUT8 host cell. In a further embodiment, the antibody, or antigen binding fragment thereof, is afucosylated at Asn297 (IgG1).

It will be apparent to those skilled in the art that such modifications may not only be used alone but may be used in combination with each other in order to further enhance effector function.

In one such embodiment, there is provided an antibody, or antigen binding fragment thereof, comprising a heavy chain constant region that comprises a both a mutated and chimeric heavy chain constant region, individually described above. For example, an antibody, or antigen binding fragment thereof, comprising at least one CH2 domain from IgG3 and one CH2 domain from IgG1, and wherein the IgG1 CH2 domain has one or more mutations at positions selected from 239, 332 and 330 (for example the mutations may be selected from S239D, I332E and A330L), such that the antibody, or antigen binding fragment thereof, has enhanced effector function, e.g. enhanced ADCC or enhanced CDC, or enhanced ADCC and enhanced CDC in comparison to an equivalent antibody, or antigen binding fragment thereof, with an IgG1 heavy chain constant region lacking said mutations. In one embodiment, the IgG1 CH2 domain has the mutations S239D and I332E. In another embodiment, the IgG1 CH2 domain has the mutations S239D, A330L, and I332E.

In an alternative embodiment, there is provided an antibody, or antigen binding fragment thereof, comprising both a chimeric heavy chain constant region and an altered glycosylation profile, as individually described above. In an embodiment, the antibody, or antigen binding fragment thereof, comprises an altered glycosylation profile such that the ratio of fucose to mannose is 0.8:3 or less. In one such embodiment, the heavy chain constant region comprises at least one CH2 domain from IgG3 and one CH2 domain from IgG1 and has an altered glycosylation profile such that the ratio of fucose to mannose is 0.8:3 or less, for example wherein the antibody, or antigen binding fragment thereof, is defucosylated. Said antibody, or antigen binding fragment thereof, has an enhanced effector function, e.g. enhanced ADCC or enhanced CDC, or enhanced ADCC and enhanced CDC, in comparison to an equivalent antibody, or antigen binding fragment thereof, with an IgG1 heavy chain constant region lacking said glycosylation profile.

In an alternative embodiment, the antibody, or antigen binding fragment thereof, has at least one IgG3 heavy chain CH2 domain and at least one heavy chain constant domain from IgG1 wherein both IgG CH2 domains are mutated in accordance with the limitations described herein.

In one aspect, there is provided a method of producing an antibody, or antigen binding fragment thereof, according to the invention described herein comprising the steps of:
a) culturing a recombinant host cell containing an expression vector comprising a nucleic acid sequence encoding a chimeric Fc domain having both IgG1 and IgG3 Fc domain amino acid residues (e.g. as described above); and wherein the FUT8 gene encoding alpha-1,6-fucosyltransferase has been inactivated in the recombinant host cell; and
b) recovering the antibody, or antigen binding fragment thereof.

Such methods for the production of an antibody, or antigen binding fragment thereof, can be performed, for example, using the ACCRETAMAB technology system available from BioWa, Inc. (Princeton, NJ) that combines the POTELLIGENT and COMPLEGENT technology systems to produce an antibody, or antigen binding fragment thereof, having both enhanced ADCC and CDC activity relative to an otherwise identical monoclonal antibody that lacks a chimeric Fc domain and that is fucosylated.

In another embodiment, there is provided an antibody, or antigen binding fragment thereof, comprising a mutated and chimeric heavy chain constant region wherein said antibody, or antigen binding fragment thereof, has an altered glycosylation profile such that the antibody, or antigen binding fragment thereof, has enhanced effector function, e.g. enhanced ADCC or enhanced CDC, or both enhanced ADCC and CDC. In one embodiment the mutations are selected from positions 239, 332 and 330, e.g. S239D, I332E and A330L. In a further embodiment the heavy chain constant region comprises at least one CH2 domain from IgG3 and one CH1 domain from IgG1. In one embodiment the heavy chain constant region has an altered glycosylation profile such that the ratio of fucose to mannose is 0.8:3 or less, e.g. the antibody, or antigen binding fragment thereof, is defucosylated, such that said antibody, or antigen binding fragment thereof, has an enhanced effector function in comparison with an equivalent non-chimeric antibody, or antigen binding fragment thereof, lacking said mutations and lacking said altered glycosylation profile.

In a further embodiment, the anti-cotinine antibody, or antigen binding fragment thereof, comprises a heavy chain CDR1 having SEQ ID NO: 1, a heavy chain CDR2 having SEQ ID NO: 2, a heavy chain CDR3 having SEQ ID NO: 3, a light chain CDR1 having SEQ ID NO: 4, a light chain CDR2 having SEQ ID NO: 5, and a light chain CDR3 having SEQ ID NO: 6. In a further embodiment, the anti-cotinine antibody has a heavy chain and a light chain, the heavy chain comprising a CDR1 having SEQ ID NO: 1, a CDR2 having SEQ ID NO: 2, and a CDR3 having SEQ ID NO: 3, and the light chain comprising a CDR1 having SEQ ID NO: 4, a CDR2 having SEQ ID NO: 5, and a CDR3 having SEQ ID NO: 6. In a further embodiment, the anti-cotinine antibody is of IgG1 isotype. In a further embodiment, the anti-cotinine antibody is of IgG1 isotype comprising a substitution in an Fc region to increase or enhance ADCC activity. In a further embodiment, the anti-cotinine antibody is of IgG1 isotype comprising a substitution in an Fc region to increase or enhance ADCC activity, wherein the substitution is S239D/I332E or S239D/I332E/A330L, wherein residue numbering is according to the EU Index. In a further embodiment, the anti-cotinine antibody is of IgG1 isotype comprising a substitution in an Fc region to increase or enhance ADCC activity, wherein the substitution is S239D/I332E, wherein residue numbering is according to the EU Index.

In a further embodiment, the anti-cotinine antibody, or antigen binding fragment thereof, comprises a heavy chain variable region (VH) having SEQ ID NO: 7, a light chain variable region (VL) having SEQ ID NO: 8. In a further embodiment, the anti-cotinine antibody has a heavy chain and a light chain, the heavy chain comprising a heavy chain variable region (VH) having SEQ ID NO: 7, and the light chain comprising a light chain variable region (VL) having SEQ ID NO: 8. In a further embodiment, the anti-cotinine antibody is of IgG1 isotype. In a further embodiment, the anti-cotinine antibody is of IgG1 isotype comprising a substitution in an Fc region to increase or enhance ADCC activity. In a further embodiment, the anti-cotinine antibody is of IgG1 isotype comprising a substitution in an Fc region to increase or enhance ADCC activity, wherein the substitution is S239D/I332E or S239D/I332E/A330L, wherein residue numbering is according to the EU Index. In a further embodiment, the anti-cotinine antibody is of IgG1 isotype comprising a substitution in an Fc region to increase or enhance ADCC activity, wherein the substitution is S239D/I332E, wherein residue numbering is according to the EU Index.

In a further embodiment, the anti-cotinine antibody has a heavy chain comprising SEQ ID NO: 9 and a light chain comprising SEQ ID NO: 10.

The present disclosure also provides a pharmaceutical composition comprising an anti-cotinine antibody, or antigen binding fragment thereof as disclosed herein, and a pharmaceutically acceptable excipient, carrier, or diluent.

The present disclosure also provides a combination comprising the compound of Formula (I) as disclosed herein, and an anti-cotinine antibody, or antigen-binding fragment thereof as disclosed herein. The compound of Formula (I) and anti-cotinine antibody, or antigen binding fragment thereof can be present in the same composition or in separate compositions. In one embodiment, a combination comprises a pharmaceutical composition comprising the compound of Formula (I) as disclosed herein and an anti-cotinine antibody, or antigen binding fragment thereof as disclosed herein, and a pharmaceutically acceptable carrier, diluent, or excipient. In another embodiment, a combination comprises a first pharmaceutical composition comprising a compound of Formula (I) as disclosed herein and a pharmaceutically acceptable carrier, diluent, or excipient; and a second pharmaceutical composition comprising an anti-cotinine antibody or antigen binding fragment thereof as disclosed herein, and a pharmaceutically acceptable carrier, excipient, or diluent.

### Statement of Use

The compounds of Formula (I) and pharmaceutically acceptable salts thereof are capable of simultaneously binding a cell surface-expressed CCR2 and an anti-cotinine antibody, or antigen binding fragment thereof to form a ternary complex for the treatment and/or prevention of diseases or disorders associated with CCR2-expressing cells.

In one embodiment, the present disclosure provides a method of treating and/or preventing a disease or disorder in a patient in need thereof comprising administering to the patient a therapeutically effective amount of the compound of Formula (I), or a pharmaceutically acceptable salt thereof, and an anti-cotinine antibody, or antigen-binding fragment thereof, wherein the disease or disorder is selected from a cancer, an inflammatory disease, an autoimmune disease, a viral infection, or a bacterial infection.

In a further embodiment, the compound and the antibody, or antigen-binding fragment thereof, are administered simultaneously. In a further embodiment, the compound and the antibody, or antigen-binding fragment thereof, are administered simultaneously from a single composition, including as a fixed-dose composition or by pre-mixing the compound and the antibody, or antigen-binding fragment thereof, prior to administration. For example, the compound and the antibody, or antigen-binding fragment thereof, can be pre-mixed about 2 seconds to about 30 seconds, about 30 seconds to about 2 minutes, about 2 minutes to about 10 minutes, about 10 minutes to about 30 minutes, or about 30 minutes to about 2 hours prior to administration. In a further embodiment, the compound and the antibody, or antigen-binding fragment thereof, are administered simultaneously from two separate compositions.

In a further embodiment, the compound and the antibody, or antigen-binding fragment thereof, are administered sequentially.

In certain embodiments, the compound and the antibody, or antigen-binding fragment thereof, whether administered simultaneously or sequentially, may be administered by the same route or may be administered by different routes. In one embodiment, the compound and the antibody, or antigen-binding fragment thereof, are both administered intraveneously or subcutaneously, in the same composition or in separate compositions. In another embodiment, the compound is administered orally and the antibody or antigen-binding fragment thereof is administered intravenously or subcutaneously.

In a further embodiment, the compound and the antibody, or antigen-binding fragment thereof, are administered in a molar ratio of compound to antibody, or antigen-binding fragment thereof, of about 2:1, about 1.8:1, about 1.6:1, about 1.5:1, about 1.4:1, about 1.3:1, about 1.2:1, about 1:1, about 1:1.2, about 1:1.3, about 1:1.4, about 1:1.5, about 1:1.6, about 1:1.8, about 1:2, about 2:1 to about 1.5:1, about 1.5:1 to about 1.2:1, about 1.2:1 to about 1:1, about 1:1 to about 1:1.2, about 1:1.2 to about 1:1.5, or about 1:1.5 to about 1:2.

In a further embodiment, the compound and the antibody, or antigen-binding fragment thereof, are present as a combination in a molar ratio of compound to antibody, or antigen-binding fragment thereof, of about 2:1, about 1.8:1, about 1.6:1, about 1.5:1, about 1.4:1, about 1.3:1, about 1.2:1, about 1:1, about 1:1.2, about 1:1.3, about 1:1.4, about 1:1.5, about 1:1.6, about 1:1.8, about 1:2, about 2:1 to about 1.5:1, about 1.5:1 to about 1.2:1, about 1.2:1 to about 1:1, about 1:1 to about 1:1.2, about 1:1.2 to about 1:1.5, or about 1:1.5 to about 1:2.

In a further embodiment, the compound and the antibody, or antigen-binding fragment thereof, are administered at a dosage of compound of 0.0001 mg/kg to 1 mg/kg and antibody of 0.01 mg/kg to 100 mg/kg. For example, in a further embodiment, the compound is administered at a dosage of about 0.0001 mg/kg to about 0.0002 mg/kg, about 0.0002 mg/kg to about 0.0003 mg/kg, about 0.0003 mg/kg to about 0.0004 mg/kg, about 0.0004 mg/kg to about 0.0005 mg/kg, about 0.0005 mg/kg to about 0.001 mg/kg, about 0.001 mg/kg to about 0.002 mg/kg, about 0.002 mg/kg to about 0.003 mg/kg, about 0.003 mg/kg to about 0.004 mg/kg, about 0.004 mg/kg to about 0.005 mg/kg, about 0.005 mg/kg to about 0.01 mg/kg, about 0.01 mg/kg to about 0.02 mg/kg, about 0.02 mg/kg to about 0.03 mg/kg, about 0.03 mg/kg to about 0.04 mg/kg, about 0.04 mg/kg to about 0.05 mg/kg, about 0.05 mg/kg to about 0.1 mg/kg, about 0.1 mg/kg to about 0.2 mg/kg, about 0.2 mg/kg to about 0.3 mg/kg, about 0.3 mg/kg to about 0.4 mg/kg, about 0.4 mg/kg to about 0.5 mg/kg, and/or about 0.5 mg/kg to about 1 mg/kg, and the antibody, or antigen-binding fragment thereof, is administered at a dosage of about 0.01 mg/kg to about 0.02 mg/kg, about 0.02 mg/kg to about 0.03 mg/kg, about 0.03 mg/kg to about 0.04 mg/kg, about 0.04 mg/kg to about 0.05 mg/kg, about 0.05 mg/kg to about 0.1 mg/kg, about 0.1 mg/kg to about 0.2 mg/kg, about 0.2 mg/kg to about 0.3 mg/kg, about 0.3 mg/kg to about 0.4 mg/kg, about 0.4 mg/kg to about 0.5 mg/kg, about 0.5 mg/kg to about 1 mg/kg, about 1 mg/kg to about 2 mg/kg, about 2 mg/kg to about 3 mg/kg, about 3 mg/kg to about 4 mg/kg, about 4 mg/kg to about 5 mg/kg, about 5 mg/kg to about 10 mg/kg, about 10 mg/kg to about 15 mg/kg, about 15 mg/kg to about 20 mg/kg, about 20 mg/kg to about 25 mg/kg, about 25 mg/kg to about 30 mg/kg, about 30 mg/kg to about 35 mg/kg, about 35 mg/kg to about 40 mg/kg, about 40 mg/kg to about 45 mg/kg, about 45 mg/kg to about 50 mg/kg, about 50 mg/kg to about 60 mg/kg, about 60 mg/kg to about 70 mg/kg, about 70 mg/kg to about 80 mg/kg, about 80 mg/kg to about 90 mg/kg, and/or about 90 mg/kg to about 100 mg/kg.

In a further embodiment, the compound and the antibody, or antigen-binding fragment thereof, are administered at a dosage of compound of 0.007 mg to 70 mg and antibody of 0.7 mg to 7000 mg. For example, in a further embodiment, the compound is administered at a dosage of about 0.007 mg to about 0.01 mg, about 0.01 mg to about 0.02 mg, about 0.02 mg to about 0.03 mg, about 0.03 mg to about 0.04 mg, about 0.04 mg to about 0.05 mg, about 0.05 mg to about 0.1 mg, about 0.1 mg to about 0.2 mg, about 0.2 mg to about 0.3 mg, about 0.3 mg to about 0.4 mg, about 0.4 mg to about 0.5 mg, about 0.5 mg to about 1 mg, about 1 mg to about 2 mg, about 2 mg to about 3 mg, about 3 mg to about 4 mg, about 4 mg to about 5 mg, about 5 mg to about 10 mg, about 10 mg to about 20 mg, about 20 mg to about 30 mg, about 30 mg to about 40 mg, about 40 mg to about 50 mg, about 50 mg to about 60 mg, and/or about 60 mg to about 70 mg, and the antibody, or antigen-binding fragment thereof, is administered at a dosage of about 0.7 mg to about 1 mg, about 1 mg to about 2 mg, about 2 mg to about 3 mg, about 3 mg to about 4 mg, about 4 mg to about 5 mg, about 5 mg to about 10 mg, about 10 mg to about 20 mg, about 20 mg to about 30 mg, about 30 mg to about 40 mg, about 40 mg to about 50 mg, about 50 mg to about 100 mg, about 100 mg to about 500 mg, about 500 mg to about 1000 mg, about 1000 mg to about 1500 mg, about 1500 mg to about 2000 mg, about 2000 mg to about 2500 mg, about 2500 mg to about 3000 mg, about 3000 mg to about 3500 mg, about 3500 mg to about 4000 mg, about 4000 mg to about 4500 mg, about 4500 mg to about 5000 mg, about 5000 mg to about 5500 mg, about 5500 mg to about 6000 mg, about 6000 mg to about 6500 mg, and/or about 6500 mg to about 7000 mg.

In a further embodiment, the compound and the antibody, or antigen-binding fragment thereof, are administered in a molar ratio and/or dosage as described herein once every week, once every two weeks, once every three weeks, once every four weeks, once every five weeks, or once every six weeks for a period of one week to one year, such as a period of one week, one month, two months, three months, four months, five months, six months, seven months, eight months, nine months, ten months, eleven months, or twelve months.

In a further embodiment, the present disclosure provides a therapeutically effective amount of the compound of Formula (I), or a pharmaceutically acceptable salt thereof, and an anti-cotinine antibody, or antigen-binding fragment thereof for use in therapy. The compound of Formula (I), or a pharmaceutically acceptable salt thereof, and anti-cotinine antibody, or antigen-binding fragment thereof can be used in treating or preventing a disease or disorder selected from a cancer, an inflammatory disease, an autoimmune disease, a viral infection, or a bacterial infection.

In a further embodiment, the present disclosure provides a therapeutically effective amount of the compound of Formula (I), or a pharmaceutically acceptable salt thereof, and an anti-cotinine antibody, or antigen-binding fragment thereof for the manufacture of a medicament. The medicament can be used in treating or preventing a disease or disorder selected from a cancer, an inflammatory disease, an autoimmune disease, a viral infection, or a bacterial infection.

In a further embodiment, the disease or disorder is mediated by chemokine receptor 2 (CCR2) and/or is associated with CCR2-positive pathogenic cells. In a further embodiment, CCR-positive cell types are identified by testing for expression of CCR by immunohistochemistry or flow cytometry.

In a further embodiment, the disease or disorder is a cancer selected from lung cancer (e.g., non-small cell lung cancer (NSCLC)), hepatocellular carcinoma (HCC), colorectal cancer (CRC), cervical cancer (e.g., cervical squamous cell carcinoma (CESC)), head and neck cancer (e.g., head and neck squamous cell carcinoma (HNSC)), pancreatic cancer, prostate cancer (e.g., metastatic castration-resistant prostate cancer (mCRPC)), ovarian cancer, endometrial cancer, bladder cancer, or breast cancer, preferably a cancer selected from NSCLC, HCC, or CRC.

In a further embodiment, the disease or disorder is a solid tumor. In a further embodiment, the disease or disorder is a solid tumor selected from lung cancer (e.g., NSCLC), HCC, CRC, cervical cancer (e.g., CESC), head and neck cancer (e.g., HNSC), pancreatic cancer, prostate cancer (e.g., mCRPC), ovarian cancer, endometrial cancer, bladder cancer, or breast cancer, preferably a solid tumor selected from NSCLC, HCC, or CRC.

In a further embodiment, the disease or disorder is a PD-1 relapsed or refractory cancer, such as a PD-1 relapsed or refractory lung cancer (e.g., NSCLC), HCC, CRC, cervical cancer (e.g., CESC), head and neck cancer (e.g., HNSC), pancreatic cancer, prostate cancer (e.g., mCRPC), ovarian cancer, endometrial cancer, bladder cancer, or breast cancer, preferably a PD-1 relapsed or refractory NSCLC, HCC, or CRC.

In a further embodiment, the disease or disorder is a non-solid cancer. In a further embodiment, the disease or disorder is a leukemia, a lymphoma, or a myeloma.

In a further embodiment, the disease or disorder is a viral infection. In a further embodiment, the viral infection is caused by an influenza virus, a coronavirus (e.g., COVID-19), or a hepatitis B virus.

In a further embodiment, the disease or disorder is a bacterial infection. In a further embodiment, the bacterial infection is a chronic bacterial infection.

In one embodiment, the present disclosure provides a method of increasing antibody-dependent cell cytotoxicity (ADCC) of CCR2-expressing cells comprising contacting the cells with an effective amount of the compound of Formula (I), or pharmaceutically acceptable salt thereof, and an anti-cotinine antibody, or antigen-binding fragment thereof, wherein the CCR2-binding moiety of the compound binds the CCR2 expressed on the cells.

In one embodiment, the present disclosure provides a method of increasing antibody dependent cellular phagocytosis (ADCP) of CCR2-expressing cells comprising contacting the cells with an effective amount of the compound of Formula (I), or pharmaceutically acceptable salt thereof, and an anti-cotinine antibody, or antigen-binding fragment thereof, wherein the CCR2-binding moiety of the compound binds the CCR2 expressed on the cells.

In one embodiment, the present disclosure provides a method of increasing complement dependant cytotoxicity (CDC) of CCR2-expressing cells comprising contacting the cells with an effective amount of the compound of Formula (I), or pharmaceutically acceptable salt thereof, and an anti-cotinine antibody, or antigen-binding fragment thereof, wherein the CCR2-binding moiety of the compound binds the CCR2 expressed on the cells.

In one embodiment, the present disclosure provides a method of conditioning a patient for therapy with a chimeric antigen receptor (CAR) T cell therapy, comprising administering to a patient an effective amount of the compound of Formula (I), or pharmaceutically acceptable salt thereof, and an anti-cotinine antibody, or antigen-binding fragment thereof. In some embodiments, the compound of Formula (I), or pharmaceutically acceptable salt thereof, and an anti-cotinine antibody, or antigen-binding fragment thereof are administered in combination with the CAR-T cell therapy. A compound of Formula (I), or pharmaceutically acceptable salt thereof, and an anti-cotinine antibody, or antigen-binding fragment thereof may be administered as a conditioning therapy or combination therapy to improve efficacy in treatment of solid tumor cancers. In other embodiments, a compound of Formula (I), or pharmaceutically acceptable salt thereof, and an anti-cotinine antibody, or antigen-binding fragment thereof may be administered as a neoadjuvant treatment for other therapies, including but not limited to immunotherapy, surgical resection, radiation, and/or chemotherapy.

In one embodiment, the present disclosure provides a method of depleting CCR2-expressing cells comprising contacting the cells with the compound of Formula (I), or pharmaceutically acceptable salt thereof, and an anti-cotinine antibody, or antigen-binding fragment thereof, wherein the CCR2-binding moiety of the compound binds the CCR2 expressed on the cells.

In a further embodiment, the CCR2-expressing cells are pathogenic cells.

In a further embodiment, the pathogenic cell is a pathogenic immune cell, a tumor cell or cancer cell, or a stromal cell.

In a further embodiment, the pathogenic immune cells are monocytes, myeloid derived suppressor cells (MDSC), such as monocytic MDSCs (mMDSCs) and polymorphonuclear MDSCs (PMN_MDSCs), T regulatory cells (Tregs), neutrophils (e.g., N2 neutrophils), macrophages (e.g., M2 macrophages), B regulatory cells (Bregs, memory B cells), plasma cells, CD8 cells (e.g., CD8 regulatory cells (CD8regs), memory CD8 cells, effector CD8 cells, naïve CD8 Tcells, TEMRA), exhausted T cells, eosinophils, basophils, mast cells, dendritic cells, natural killer (NK cells), innate lymphoid cells, NK T cells (NKT), or γδT cells.

In a further embodiment, the pathogenic immune cells are myeloid derived suppressor cells (MDSC), such as monocytic MDSCs (mMDSCs) and polymorphonuclear MDSCs (PMN_MDSCs), T regulatory cells (Tregs), neutrophils (e.g., N2 neutrophils), macrophages (e.g., M2 macrophages), B regulatory cells (Bregs), CD8 regulatory cells (CD8regs), exhausted T cells.

In a further embodiment, the tumor cells or cancer cells are lung cancer cells (e.g., non-small cell lung cancer (NSCLC) cells), hepatocellular carcinoma (HCC) cells, colorectal cancer (CRC) cells, cervical cancer cells (e.g., cervical squamous cell carcinoma (CESC) cells), head and neck cancer cells (e.g., head and neck squamous cell carcinoma (HNSC) cells), pancreatic cancer cells, prostate cancer cells (e.g., metastatic castration-resistant prostate cancer (mCRPC) cells), ovarian cancer cells, endometrial cancer cells, bladder cancer cells, or breast cancer cells, preferably NSCLC cells, HCC cells, or CRC cells.

In a further embodiment, the stromal cells are cancer associated fibroblasts (CAFs).

### Combination Therapies

The compounds of the invention may be employed alone or in combination with other therapeutic agents. Combination therapies according to the present invention thus comprise the administration of at least one compound of Formula (I) or a pharmaceutically acceptable salt thereof, and the use of at least one other pharmaceutically active agent. The compounds of the invention and the other pharmaceutically active agents may be administered together in a single pharmaceutical composition or separately and, when administered separately this may occur simultaneously or sequentially in any order. The amounts of the compounds of the invention and the other pharmaceutically active agents and the relative timings of administration will be selected in order to achieve the desired combined therapeutic effect.

It will be appreciated that when the compound of the present invention is administered in combination with one or more other therapeutically active agents normally administered by the inhaled, intravenous, oral, intranasal, ocular topical or other route, that the resultant pharmaceutical composition may be administered by the same route. Alternatively, the individual components of the composition may be administered by different routes.

In one embodiment, the compounds and pharmaceutical composition disclosed herein are used in combination with, or include, one or more additional therapeutic agents. In a further embodiment, the additional therapeutic agent is a checkpoint inhibitor or an immune modulator.

In a further embodiment, the checkpoint inhibitor is selected from a PD-1 inhibitor (e.g., an anti-PD-1 antibody including, but not limited to, pembrolizumab, nivolumab, cemiplimab, or dostarlimab), a PD-L1 inhibitor (e.g., an anti-PD-L1 antibody including, but not limited to, atezolizumab, avelumab, or durvalumab), or a CTLA-4 inhibitor (e.g., an anti-CTLA-4 antibody including, but not limited to, ipilimumab or tremilumumab).

In a further embodiment, the checkpoint inhibitor is selected from a CD226 axis inhibitor, including but not limited to a TIGIT inhibitor (e.g., an anti-TIGIT antibody), a CD96 inhibitor (e.g., an anti-CD96 antibody), and/or a PVRIG inhibitor (e.g., an anti-PVRIG antibody).

In a further embodiment, the immune modulator is an ICOS agonist (e.g., an anti-ICOS antibody including, but not limited to feladilimab), a PARP inhibitor (e.g., niraparib, olaparib), or a STING agonist.

### Pharmaceutical Compositions, Dosages, and Dosage Forms

For the purposes of administration, in certain embodiments, the ARMs described herein are administered as a raw chemical or are formulated as pharmaceutical compositions. Pharmaceutical compositions disclosed herein include an ARM and one or more of: a pharmaceutically acceptable carrier, diluent or excipient. An ARM is present in the composition in an amount which is effective to treat a particular disease, disorder or condition of interest. The activity of the ARM can be determined by one skilled in the art, for example, as described in the biological assays described below. Appropriate concentrations and dosages can be readily determined by one skilled in the art. In certain embodiments, the ARM is present in the pharmaceutical composition in an amount from about 25 mg to about 500 mg. In certain embodiments, the ARM is present in the pharmaceutical composition in an amount of about 0.01 mg to about 300 mg. In certain embodiments, ARM is present in the pharmaceutical composition in an amount of about 0.01 mg, 0.1 mg, 1 mg, 5 mg, 10 mg, 25 mg, 50 mg, 100 mg, 200 mg, 300 mg, 400 mg or about 500 mg.

Administration of the compounds of the invention, or their pharmaceutically acceptable salts, in pure form or in an appropriate pharmaceutical composition, is carried out via any of the accepted modes of administration of agents for serving similar utilities. The pharmaceutical compositions of the invention are prepared by combining a compound of the invention with an appropriate pharmaceutically acceptable carrier, diluent or excipient, and in specific embodiments are formulated into preparations in solid, semi-solid, liquid or gaseous forms, such as tablets, capsules, powders, granules, ointments, solutions, suppositories, injections, inhalants, gels, microspheres, and aerosols. Exemplary routes of administering such pharmaceutical compositions include, without limitation, oral, topical, transdermal, inhalation, parenteral (e.g., intramuscular, subcutaneous, intravenous, or intradermal), sublingual, buccal, rectal, vaginal, and intranasal. Pharmaceutical compositions of the invention are formulated so as to allow the active ingredients contained therein to be bioavailable upon administration of the composition to a patient.

Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington: The Science and Practice of Pharmacy, 20th Edition (Philadelphia. College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings described herein.

The pharmaceutical compositions disclosed herein are prepared by methodologies well known in the pharmaceutical art. For example, in certain embodiments, a pharmaceutical composition intended to be administered by injection is prepared by combining a compound of the invention with sterile, distilled water so as to form a solution. In some embodiments, a surfactant is added to facilitate the formation of a homogeneous solution or suspension. Surfactants are compounds that non-covalently interact with the compound of the invention so as to facilitate dissolution or homogeneous suspension of the compound in the aqueous delivery system.

Traditional antibody therapeutics have several disadvantages that are addressed by the ARMs approach described herein including difficulties in managing adverse events via adjusting dose and dose frequency of administration, challenges in generating antibodies to certain classes of drug targets (e.g., GPCRs, ion channels, and enzymes), and a new cell line for development is required for each new antibody which can be slow and costly. Moreover, different formats of biologics (e.g., bispecifics) can be challenging to manufacture. In contrast, the ARMs approach provides the following advantages: uniting the pharmacology of antibodies with the dose-control of small molecules, dose controlled PK/PD allowing temporal cell depletion, simpler multimerization, and rapid reversal of cell depletion through dosing of the antibody-binding component (e.g., cotinine hapten) which can uncouple therapeutic effects from potential adverse events.

### EXAMPLES

The following examples illustrate the invention. These Examples are not intended to limit the scope of the invention, but rather to provide guidance to the skilled artisan to prepare and use the compounds, compositions, and methods of the invention. While particular embodiments of the invention are described, the skilled artisan will appreciate that various changes and modifications can be made. References to preparations carried out in a similar manner to, or by the general method of, other preparations, may encompass variations in routine parameters such as time, temperature, workup conditions, minor changes in reagent amounts etc. Chemical names for all title compounds were generated using ChemDraw Plug-in version 16.0.1.13c (90) or ChemDraw desktop version 16.0.1.13 (90). A person of ordinary skill in the art will recognize that compounds of the invention may have alternative names when different naming software is used.

### Compound Synthesis

The compounds according to Formula (I) are prepared using conventional organic synthetic methods. A suitable synthetic route is depicted below in the following general reaction schemes. All the starting materials are commercially available or are readily prepared from commercially available starting materials by those of skill in the art.

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Organic Synthesis (4th ed.), John Wiley & Sons, NY (2006). In some instances, a substituent may be specifically selected to be reactive under the reaction conditions used. Under these circumstances, the reaction conditions convert the selected substituent into another substituent that is either useful as an intermediate compound or is a desired substituent in a target compound.

### Intermediate 1—2,4-Dichloro-6-(trifluoromethyl)quinazoline

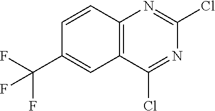 [SMILES: FC(F)(F)c1ccc2nc(Cl)nc(Cl)c2c1]

### Step 1: 2 6-(Trifluoromethyl)quinazoline-2,4(1H,3H)-dione

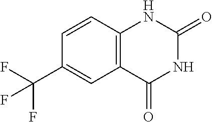 [SMILES: O=c1[nH]c(=O)c2cc(C(F)(F)F)ccc2[nH]1]

To a stirred suspension of 2-amino-5-(trifluoromethyl)benzoic acid (5 g, 24.37 mmol) in water (150 mL) was added acetic acid (4.19 mL, 73.1 mmol) and the mixture was stirred at 50° C. for 30 min. A solution of potassium cyanate (4.94 g, 60.9 mmol) in water (50 mL) was added dropwise and the mixture was stirred at 50° C. for 1 h, was cooled to rt, and a solution of sodium hydroxide (43.9 g, 1097 mmol) in water (100 mL) was added. The mixture was stirred for 30 min and the pH was adjusted to 4 using concentrated HCl, which was slowly added. The obtained solid was filtered, was washed with ice-cold water, and was dried under reduced pressure to provide the title compound as an off-white solid (2.6 g, 10.67 mmol, 43.8% yield. LC-MS m/z 228.9 (M+H)⁺.

### Step 2: 2,4-Dichloro-6-(trifluoromethyl)quinazoline

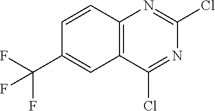 [SMILES: FC(F)(F)c1ccc2nc(Cl)nc(Cl)c2c1]

To 6-(trifluoromethyl)quinazoline-2,4(1H,3H)-dione (2.6 g, 11.30 mmol) was added N,N-dimethylaniline (0.143 mL, 1.130 mmol) and phosphoryl chloride (26.3 mL, 282 mmol) at rt under an atmosphere of nitrogen. The mixture was stirred at 120° C. for 16 h. Phosphoryl chloride was distilled from the reaction and the pH of the was adjusted to ˜8 using aqueous NaHCO₃. The mixture was extracted with ethyl acetate (2×60 mL), was dried out with Na₂SO₄, was filtered, and the filtrate was concentrated under reduced pressure. Purification by normal-phase chromatography (silica gel 60-120 mesh) eluting with ethyl acetate in hexane (5%) provided the title compound as a brown solid (2.1 g, 6.31 mmol, 55.8% yield). The material was combined with two other batches and was purified by normal-phase chromatography (silica gel 60-120 mesh) eluting with ethyl acetate in hexane (5%) to provide the title compound as a pale-yellow solid (7.2 g, 6.5 mmol, 54.4% yield). LC-MS m/z 267.3 (M+H)⁺ and 269.3 (M+H)⁺.

### Intermediate 2—2-(3-(4-Chloro-6-(trifluoromethyl)quinazolin-2-yl)propyl) isoindoline-1,3-dione

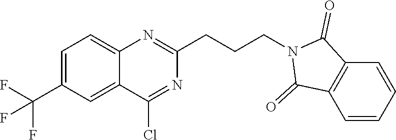 [SMILES: O=C1c2ccccc2C(=O)N1CCCc1nc(Cl)c2cc(C(F)(F)F)ccc2n1]

### Step 1: 4-(1,3-Dioxoisoindolin-2-yl)butanenitrile

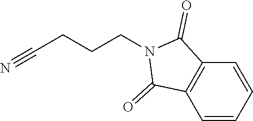 [SMILES: N#CCCCN1C(=O)c2ccccc2C1=O]

A mixture of 4-bromobutanenitrile (5.05 g, 34.1 mmol), phthalimide potassium salt (7.02 g, 37.9 mmol) and potassium iodide (0.085 g, 0.512 mmol) in N,N-dimethylformamide (DMF) (40 mL) was heated at 80 to 90° C. for 5 h, was cooled, and was poured into water (200 g). The mixture was extracted with CHCl₃ (3×60 mL). The combined organic extracts were washed with sodium hydroxide (0.5%), with brine, were dried over MgSO₄, and were concentrated under reduced pressure to provide the title compound as yellow liquid (8.803 g, 32.9 mmol, 96% yield). LC-MS m/z 215.1 (M+H)⁺.

### Step 2: 2-(3-(4-Oxo-6-(trifluoromethyl)-3,4-dihydroquinazolin-2-yl)propyl) isoindoline-1,3-dione

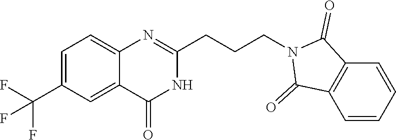 [SMILES: O=C1c2ccccc2C(=O)N1CCCc1nc2ccc(C(F)(F)F)cc2c(=O)[nH]1]

To a mixture of methyl 2-amino-5-(trifluoromethyl)benzoate (3.615 g, 16.49 mmol) in 4 M HCl in 1,4-dioxane (41.2 mL, 165 mmol) was added 4-(1,3-dioxoisoindolin-2-yl)butanenitrile (8.8 g, 32.9 mmol). The mixture was stirred at 60 to 65° C. overnight in a sealed tube and was cooled to rt. The white precipitate was collected by filtration, washed with ethanol and 1,4-dioxane, and was dried to provide the title compound (4.641 g, 10.99 mmol, 66.6% yield). LC-MS m/z 402.0 (M+H)⁺.

### Step 3: 2-(3-(4-Chloro-6-(trifluoromethyl)quinazolin-2-yl)propyl)isoindoline-1,3-dione

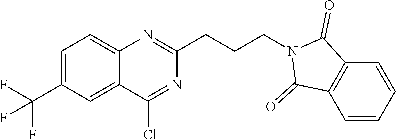 [SMILES: O=C1c2ccccc2C(=O)N1CCCc1nc(Cl)c2cc(C(F)(F)F)ccc2n1]

To a mixture of 2-(3-(4-oxo-6-(trifluoromethyl)-1,4-dihydroquinazolin-2-yl)propyl) isoindoline-1,3-dione (2.32 g, 5.78 mmol) and N,N-diisopropylethylamine (6.06 mL, 34.7 mmol) in toluene (50 mL) was added phosphoryl chloride (3.04 mL, 32.6 mmol). The mixture was stirred at 100 to 110° C. overnight. Phosphoryl chloride (3 mL, 32.6 mmol) was added and the mixture was stirred at 100 to 110° C. over the weekend. The mixture was cooled to rt and was concentrated under reduced pressure. The residue was partitioned between dichloromethane (DCM) (150 mL) and NaHCO₃ (2%) (100 mL). The organic phase was washed with brine, was dried over MgSO₄, and was concentrated. Purification by ISCO CombiFlash® chromatography (80 g ISCO Gold silica column, 65 mL/min) eluting with a gradient of 10 to 50% ethyl acetate in heptane provided the title compound as a pale-yellow solid (1.233 g, 2.88 mmol, 49.8% yield). LC-MS m/z 420.0 (M+H)⁺.

The following intermediates were or could be prepared using procedures analogous to those described for Intermediate 2:

| | | | LC-MS |
|---|---|---|---|
| Intermediate | Compound | Structure | m/z |
| 3 | 2-(2-(4-Chloro-6- (trifluoromethyl)- quinazolin-2-yl)- ethyl)isoindoline- 1,3-dione | 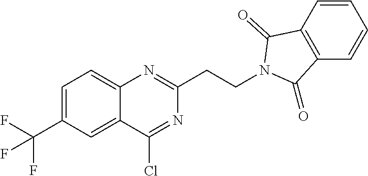 [SMILES: O=C1c2ccccc2C(=O)N1CCc1nc(Cl)c2cc(C(F)(F)F)ccc2n1] | 406.3 (M + H)⁺ |
| 4 | 2-(4-(4-Chloro-6- (trifluoromethyl)- quinazolin-2-yl)- butyl)isoindoline- 1,3-dione | 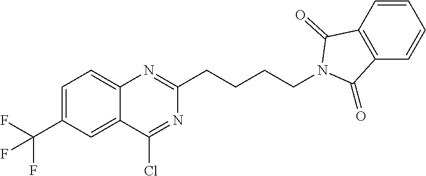 [SMILES: O=C1c2ccccc2C(=O)N1CCCCc1nc(Cl)c2cc(C(F)(F)F)ccc2n1] | 434.4 (M + H)⁺ |
| 5 | 2-(2-((4-Chloro-6- (trifluoromethyl)- quinazolin-2-yl)- methoxy)ethyl)- isoindoline-1,3- dione | 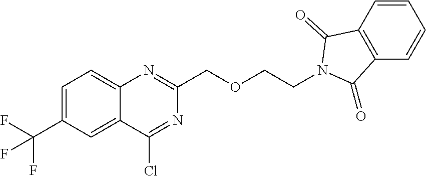 [SMILES: O=C1c2ccccc2C(=O)N1CCOCc1nc(Cl)c2cc(C(F)(F)F)ccc2n1] | 436.2 (M + H)⁺ |

### Intermediate 6—tert-Butyl 4-((4-(butylsulfinyl)-6-(trifluoromethyl)quinazolin-2-yl)oxy)piperidine-1-carboxylate

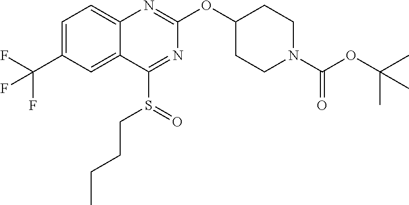 [SMILES: CCCCS(=O)c1nc(OC2CCN(C(=O)OC(C)(C)C)CC2)nc2ccc(C(F)(F)F)cc12]

### Step 1: 4-(Butylthio)-2-chloro-6-(trifluoromethyl)quinazoline

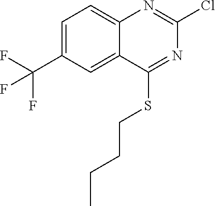 [SMILES: CCCCSc1nc(Cl)nc2ccc(C(F)(F)F)cc12]

To solution of 2,4-dichloro-6-(trifluoromethyl)quinazoline (0.9895 g, 3.71 mmol) in tetrahydrofuran (THF) (20 mL) at 0° C. was added sodium butane-1-thiolate (0.499 g, 4.45 mmol) and the mixture was stirred at rt overnight. 1 N NaOH was added and the mixture was extracted with ethyl acetate. The combined organic extracts were washed with brine, were dried over MgSO₄, and were filtered, ISOLUTE® absorbent was added, and the filtrate was concentrated under reduced pressure. Purification by normal-phase chromatography (40 g silica) eluting with a gradient of from 0 to 20% ethyl acetate in heptane provided the title compound as a white solid (942.0 mg, 2.94 mmol, 79% yield). LC-MS m/z 321.2 (M+H)⁺.

### Step 2: tert-Butyl 4-((4-(butylthio)-6-(trifluoromethyl)quinazolin-2-yl)oxy) piperidine-1-carboxylate

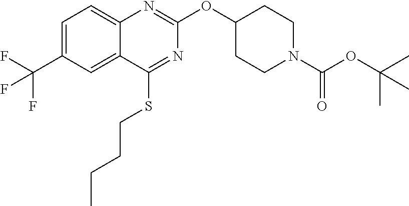 [SMILES: CCCCSc1nc(OC2CCN(C(=O)OC(C)(C)C)CC2)nc2ccc(C(F)(F)F)cc12]

To a solution of tert-butyl 4-hydroxypiperidine-1-carboxylate (31.4 mg, 0.156 mmol) in N,N-dimethylformamide (DMF) (1 mL) at 0° C. was added 60% sodium hydride in mineral oil (6.23 mg, 0.156 mmol) and the mixture was stirred for 15 min. 4-(Butylthio)-2-chloro-6-(trifluoromethyl)quinazoline (25 mg, 0.078 mmol) was added, the mixture was stirred for 20 min, and was warmed to rt. Water was added and the mixture was combined with the crude product from a similar batch. The aqueous phase was extracted with ethyl acetate (2×). The combined organic phases were washed with brine, were dried over MgSO₄, were filtered, ISOLUTE® absorbent was added, and the filtrate was concentrated under reduced pressure. Purification by normal-phase chromatography (12 g silica) eluting with a gradient of from 0 to 20% ethyl acetate in heptane provided the title compound as a clear, colorless oil (36.0 mg, 0.074 mmol, 95% yield). LC-MS m/z 486.6 (M+H)⁺.

### Step 3: tert-Butyl 4-((4-(butylsulfinyl)-6-(trifluoromethyl)quinazolin-2-yl)oxy) piperidine-1-carboxylate

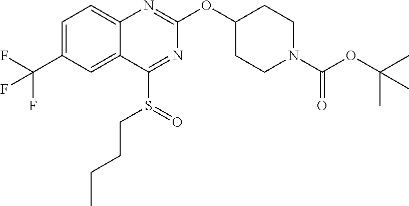 [SMILES: CCCCS(=O)c1nc(OC2CCN(C(=O)OC(C)(C)C)CC2)nc2ccc(C(F)(F)F)cc12]

To a solution of tert-butyl 4-((4-(butylthio)-6-(trifluoromethyl)quinazolin-2-yl)oxy)piperidine-1-carboxylate (766.1 mg, 1.578 mmol) in ethanol (15 mL) was added magnesium monoperoxyphthalate (MMPP) (468 mg, 0.947 mmol). The mixture was stirred at rt for 1 h and was concentrated under reduced pressure. The residue was partitioned between water and ethyl acetate. The organic phase was dried over MgSO₄, was filtered, ISOLUTE® absorbent was added, and the filtrate was concentrated under reduced pressure. Purification by normal-phase chromatography (40 g silica) eluting with a gradient of from 0 to 100% ethyl acetate in heptane provided the title compound as a clear oil (600 mg, 1.196 mmol, 76% yield). LC-MS m/z 502.4 (M+H)⁺.

The following intermediates were or could be prepared using procedures analogous to those described for Intermediate 6:

| | | | LC-MS |
|---|---|---|---|
| Intermediate | Compound | Structure | m/z |
| 7 | tert-Butyl 4-(((4-(butyl- sulfinyl)-6-(trifluoro- methyl)quinazolin-2- yl)oxy)methyl)piperi- dine-1-carboxylate | 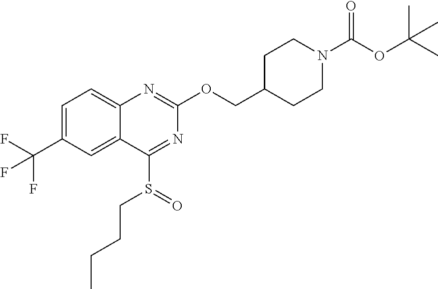 [SMILES: CCCCS(=O)c1nc(OCC2CCN(C(=O)OC(C)(C)C)CC2)nc2ccc(C(F)(F)F)cc12] | 516.4 (M + H)⁺ |

### Intermediate 8—N-((1R,2S,5R)-2-((S)-3-Amino-2-oxopyrrolidin-1-yl)-5-(tert-butylamino) cyclohexyl)acetamide

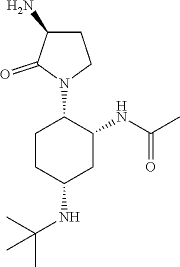 [SMILES: CC(=O)N[C@@H]1C[C@H](NC(C)(C)C)CC[C@@H]1N1CC[C@H](N)C1=O]

### Step 1: Ethyl 8-oxo-1,4-dioxaspiro[4.5]decane-7-carboxylate

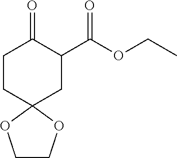 [SMILES: CCOC(=O)C1CC2(CCC1=O)OCCO2]

To a suspension of sodium hydride (366 g, 9156 mmol) in tetrahydrofuran (THF) (5000 mL) stirred under nitrogen at rt was added diethyl carbonate (983 g, 8324 mmol) and the mixture was heated to 60° C. 1,4-Dioxaspiro[4.5]decan-8-one (650 g, 4162 mmol) in tetrahydrofuran (THF) (3000 mL) solution was added in dropwise over 1 h and the mixture was stirred for 4 h. The mixture was cooled to 0° C., ice cold water (10 L) and acetic acid (850 mL) were added, and the aqueous phase was extracted with n-heptane (3×1500 mL). The combined organic phases were washed with saturated NaHCO₃ (1000 mL), with brine (1000 mL), were dried over the Na₂SO₄, and were concentrated under the reduced pressure. The residue was triturated with n-hexane (100 mL) and maintained at 0° C. for 6 h, was collected by filtration, and was washed with n-hexane (50 mL) to provide the title compound as an off-white solid (580 g, 2493 mmol, 59.9% yield). LC-MS m/z 229.0 (M+H)⁺.

### Step 2: Ethyl 8-oxo-1,4-dioxaspiro[4.5]decane-7-carboxylate

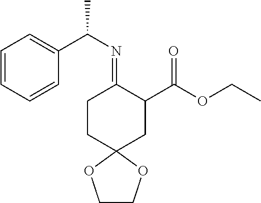 [SMILES: CCOC(=O)C1CC2(CC/C1=N\[C@@H](C)c1ccccc1)OCCO2]

To a stirred solution of ethyl 8-oxo-1,4-dioxaspiro[4.5]decane-7-carboxylate (1255 g, 5499 mmol) and acetic acid (15.74 mL, 275 mmol) in methanol (10000 mL) under an atmosphere of nitrogen at rt was added commercially available (S)-1-phenylethan-1-amine (851 mL, 6598 mmol) and the mixture heated at 55° C. overnight. The mixture was concentrated under reduced pressure. The residue was washed with petroleum ether (2000 mL), with water (4×1000 mL), and was dried under reduced pressure to provide the title compound as an off-white solid (1330 g, 3973 mmol, 72.3% yield). LC-MS m/z 332.2 (M+H)⁺.

### Step 3: Ethyl (7R,8S)-8-(((S)-1-phenylethyl)amino)-1,4-dioxaspiro[4.5]decane-7-carboxylate

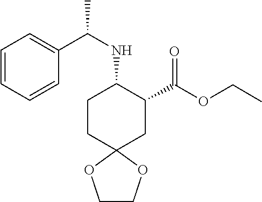 [SMILES: CCOC(=O)[C@@H]1CC2(CC[C@@H]1N[C@@H](C)c1ccccc1)OCCO2]

To a solution of ethyl (E)-8-(((S)-1-phenylethyl)imino)-1,4-dioxaspiro[4.5]decane-7-carboxylate (300 g, 905 mmol) in ethanol (2000 mL) and ethyl acetate (1250 mL) were added 10% Pt/C (25 g, 12.82 mmol) and acetic acid (119 mL, 2082 mmol) under an atmosphere of nitrogen at rt. The mixture was stirred in autoclave at 40° C. under 50 psi of hydrogen for 16 h. The mixture was filtered through Celite®, the catalyst was washed with ethyl acetate (2×500 mL), and the filtrate was concentrated under reduced pressure. The residue was dissolved in ethyl acetate, was washed with saturated NaHCO₃, and with brine. The organic phase was concentrated under the reduced pressure to provide the title compound as a yellow gum (230 g, 643 mmol, 71.0% yield) as yellow gum. LC-MS m/z 334.2 (M+H)⁺.

### Step 4: (7R,8S)-7-(Ethoxycarbonyl)-N—((S)-1-phenylethyl)-1,4-dioxaspiro[4.5]decan-8-aminium 4-methylbenzenesulfonate

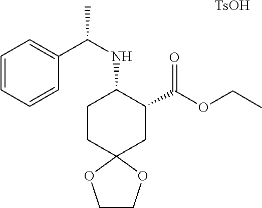 [SMILES: CCOC(=O)[C@@H]1CC2(CC[C@@H]1N[C@@H](C)c1ccccc1)OCCO2 ; "TsOH"]

To a stirred solution of ethyl (7R,8S)-8-(((S)-1-phenylethyl)amino)-1,4-dioxaspiro[4.5]decane-7-carboxylate (900 g, 2699 mmol) in tetrahydrofuran (THF) (4000 mL) under an atmosphere of nitrogen at 0° C. was added dropwise a solution of p-toluenesulfonic acid monohydrate (565 g, 2969 mmol) in tetrahydrofuran (THF) (1000.0 mL) over 1 h. The resultant slurry was stirred at rt for 16 h. The precipitate was collected by filtration, was rinsed with methyl t-butyl ether (1000.0 mL) and was dried under reduced pressure to provide the title compound (480 g, 947 mmol, 35.1% yield). The filtrate was concentrated under reduced pressure and the residue was stirred in diethyl ether (1000 mL) for 15 min. The solvent was decanted, ethyl acetate (1000 mL) was added, the mixture was stirred for 30 min. The solid was collected by filtration and was rinsed with diethyl ether (1000 mL) to provide the title compound as a white solid (480 g, 947 mmol, 35.1% yield). LC-MS m/z 334.2 (M+H)⁺. The peak for p-toluenesulfonic acid was also observed, LC-MS m/z 171.0 (M−H)⁺.

### Step 5: (7R,8S)-7-(Ethoxycarbonyl)-1,4-dioxaspiro[4.5]decan-8-aminium 4-methylbenzenesulfonate

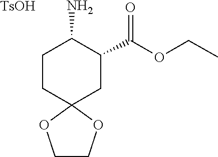 [SMILES: "TsOH" ; CCOC(=O)[C@@H]1CC2(CC[C@@H]1N)OCCO2]

To the solution of (7R,8S)-7-(ethoxycarbonyl)-N—((S)-1-phenylethyl)-1,4-dioxaspiro[4.5]decan-8-aminium 4-methylbenzenesulfonate (240 g, 475 mmol) in ethanol (2 L) and ethyl acetate (1 L) was added 10% Pd—C(50 g, 47.0 mmol) under an atmosphere of nitrogen and the mixture was stirred under hydrogen (8 Kg/cm2) at 50° C. for 20 h. The mixture was filtered through Celite® and the catalyst was washed with ethyl acetate (1 L). The filtrate was concentrated under reduced pressure to provide the title compound as a pale-yellow, sticky oil (180 g, 436 mmol, 92% yield). LC-MS m/z 230.1 (M+H)⁺.

### Step 6: Ethyl (7R,8S)-8-((S)-2-(((benzyloxy)carbonyl)amino)-4-(methylthio) butanamido)-1,4-dioxaspiro[4.5]decane-7-carboxylate

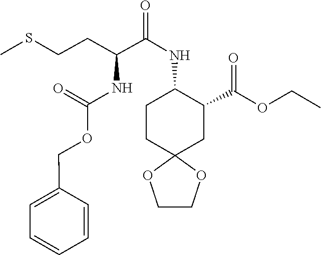 [SMILES: CCOC(=O)[C@@H]1CC2(CC[C@@H]1NC(=O)[C@H](CCSC)NC(=O)OCc1ccccc1)OCCO2]

To a solution of (7R,8S)-7-(ethoxycarbonyl)-1,4-dioxaspiro[4.5]decan-8-aminium 4-methylbenzenesulfonate (360 g, 897 mmol) in acetonitrile (3.6 L) was added ((benzyloxy)carbonyl)-L-methionine (267 g, 942 mmol), EDC (189 g, 986 mmol) and HOBt (165 g, 1076 mmol) under an atmosphere of nitrogen at 0° C. DIPEA (0.345 L, 1973 mmol) was added dropwise over 60 min and the reaction was stirred at rt for 16 h. Ice-cold water (2000 mL) was added and the aqueous phase was extracted with ethyl acetate (3×2000 mL). The combined organic phases were washed with saturated NaHCO₃ (1000 mL), with 1.5 M HCl (1000 mL), and with brine (1000 mL), were dried over Na₂SO₄ (500 g), and were concentrated under reduced pressure. The residue was triturated with petroleum ether (2×500 mL). The solid was collected by filtration, was washed with petroleum ether, and was dried under reduced pressure to provide the title compound as an off-white solid (360 g, 645 mmol, 71.9% yield). LC-MS m/z 495.2 (M+H)⁺.

### Step 7: ((S)-3-(((Benzyloxy)carbonyl)amino)-4-(((7R,8S)-7-(ethoxycarbonyl)-1,4-dioxaspiro[4.5]decan-8-yl)amino)-4-oxobutyl)dimethylsulfonium iodide

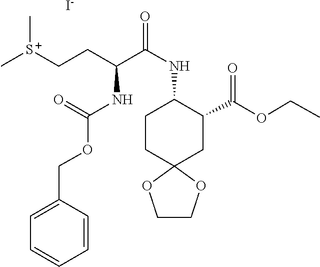 [SMILES: [I-] ; CCOC(=O)[C@@H]1CC2(CC[C@@H]1NC(=O)[C@H](CC[S+](C)C)NC(=O)OCc1ccccc1)OCCO2]

To a stirred solution of ethyl (7R,8S)-8-((S)-2-(((benzyloxy)carbonyl)amino)-4-(methylthio)butanamido)-1,4-dioxaspiro[4.5]decane-7-carboxylate (360 g, 728 mmol) in ethyl acetate (3.6 L) under an atmosphere of nitrogen at 0° C. was added dropwise iodomethane (0.683 L, 10900 mmol) over 30 min and reaction was stirred at rt for 18 h. Methyl t-butyl ether (2500 mL) was added and the mixture was stirred for 1 h. The solid was collected by filtration, was washed with methyl t-butyl ether (1500 mL), and was dried to provide the title compound as an off-white solid. LC-MS m/z 447.2 (M+H)⁺¹H NMR (400 MHz, CHLOROFORM-d) δ ppm 7.26-7.44 (m, 5H) 5.00-5.20 (s, 2H) 4.69 (d, J=4.6 Hz, 1H) 4.41 (br s, 1H) 3.46-4.17 (m, 6H) 3.01-3.27 (m, 5H) 2.75-2.94 (m, 1H) 2.44 (br s, 1H) 1.49-2.32 (m, 6H) 1.11-1.31 (m, 8H).

### Step 8: Ethyl (7R,8S)-8-((S)-3-(((benzyloxy)carbonyl)amino)-2-oxopyrrolidin-1-yl)-1,4-dioxaspiro[4.5]decane-7-carboxylate

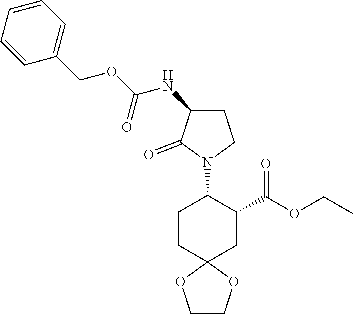 [SMILES: CCOC(=O)[C@@H]1CC2(CC[C@@H]1N1CC[C@H](NC(=O)OCc3ccccc3)C1=O)OCCO2]

To the stirred solution of ((S)-3-(((benzyloxy)carbonyl)amino)-4-(((7R,8S)-7-(ethoxycarbonyl)-1,4-dioxaspiro[4.5]decan-8-yl)amino)-4-oxobutyl)dimethylsulfonium iodide (360 g, 566 mmol) in dimethyl sulfoxide (DMSO) (3.6 L) was added cesium carbonate (203 g, 622 mmol) at 0° C. and the mixture was stirred at 20° C. for 18 h. Ice-cold water (5 L) was added and the mixture was extracted with ethyl acetate (3×3 L). The combined organic extracts were washed with water (3×3 L), with brine (1×2 L), were dried over Na₂SO₄ (200 g), and were concentrated under reduced pressure. The residue was recrystallized with diethyl ether (1 L) and was collected by filtration. The solid was washed with diethyl ether (500 mL) and was dried under reduced pressure to provide the title compound as an off-white solid (155 g, 347 mmol, 61.4% yield). LC-MS m/z 447.2 (M+H)⁺.

### Step 9: Ethyl (1R,2S)-2-((S)-3-(((benzyloxy)carbonyl)amino)-2-oxopyrrolidin-1-yl)-5-oxocyclohexane-1-carboxylate

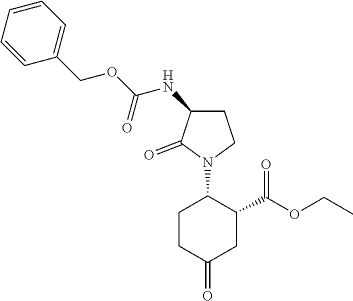 [SMILES: CCOC(=O)[C@@H]1CC(=O)CC[C@@H]1N1CC[C@H](NC(=O)OCc2ccccc2)C1=O]

5 M HCl (104 mL, 521 mmol) was added dropwise over 20 min to a stirred solution of ethyl (7R,8S)-8-((S)-3-(((benzyloxy)carbonyl)amino)-2-oxopyrrolidin-1-yl)-1,4-dioxaspiro[4.5]decane-7-carboxylate (155 g, 347 mmol) in acetone (1550 mL) under an atmosphere of nitrogen at 55° C. and the mixture was stirred at 55° C. for 4 h. The mixture was cooled to rt and the solvent was evaporated under reduced pressure. Water (500 mL) was added, the solid was collected by filtration, was washed with water (50 mL), and was dried under reduced pressure to provide the title compound as an off-white solid (130 g, 317 mmol, 91% yield). LC-MS m/z 403.2 (M+H)⁺.

### Step 10: (1R,2S)-2-((S)-3-(((Benzyloxy)carbonyl)amino)-2-oxopyrrolidin-1-yl)-5-oxocyclohexane-1-carboxylic acid

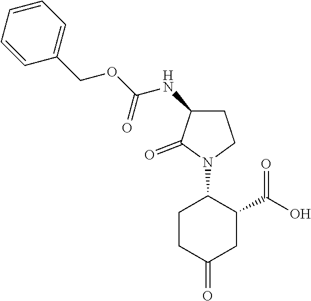 [SMILES: O=C1CC[C@H](N2CC[C@H](NC(=O)OCc3ccccc3)C2=O)[C@H](C(=O)O)C1]

To a stirred solution of ethyl (1R,2S)-2-((S)-3-(((benzyloxy)carbonyl)amino)-2-oxopyrrolidin-1-yl)-5-oxocyclohexane-1-carboxylate (130 g, 323 mmol) in tetrahydrofuran (THF) (900 mL) at 0° C. was added dropwise a solution of sodium hydroxide (25.8 g, 646 mmol) in water (450 mL) over 10 min and the mixture was stirred at 0° C. for 3 h. Water (250 mL) and ethyl acetate (250 mL) were added and the mixture was stirred for 10 min. The aqueous phase was cooled to 0° C. and the pH was adjusted to 4 to 5 by slowing adding 5 N HCl (70 mL). The aqueous phase was extracted with dichloromethane (DCM) (3×200 mL). The combined organic phases were washed with brine (100 mL), were dried over Na₂SO₄, and were concentrated under reduced pressure. The residue was triturated with diethyl ether (100 mL) and the solid was dried under reduced pressure to provide the title compound as an off-white, sticky solid (110 g, 291 mmol, 90% yield). LC-MS m/z 375.2 (M+H)⁺.

### Step 11: (7R,8S)-8-((S)-3-(((Benzyloxy)carbonyl)amino)-2-oxopyrrolidin-1-yl)-1,4-dioxaspiro[4.5]decane-7-carboxylic acid

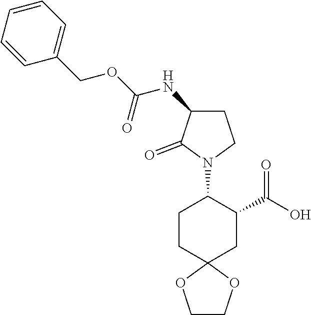 [SMILES: O=C(N[C@H]1CCN([C@H]2CCC3(C[C@H]2C(=O)O)OCCO3)C1=O)OCc1ccccc1]

To a stirred solution of (1R,2S)-2-((S)-3-(((benzyloxy)carbonyl)amino)-2-oxopyrrolidin-1-yl)-5-oxocyclohexane-1-carboxylic acid (300 g, 801 mmol) in toluene (3000 mL) under an atmosphere of nitrogen at rt was added ethylene glycol (268 mL, 4808 mmol). The mixture was stirred at 50° C. for 3 h and was concentrated under reduced pressure. The residue was dissolved in dichloromethane (DCM) (2000 mL). The organic phase was washed with water (500 mL), with brine (500 mL), was dried over Na₂SO₄, and was concentrated under reduced pressure to provide the title compound as a gum (280 g, 404 mmol, 50.4% yield). LC-MS m/z 419.1 (M+H)⁺.

### Step 12: Benzyl ((S)-1-((7R,8S)-7-acetamido-1,4-dioxaspiro[4.5]decan-8-yl)-2-oxopyrrolidin-3-yl)carbamate

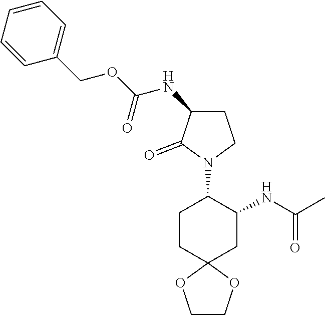 [SMILES: CC(=O)N[C@@H]1CC2(CC[C@@H]1N1CC[C@H](NC(=O)OCc3ccccc3)C1=O)OCCO2]

To a stirred solution of (7R,8S)-8-((S)-3-(((benzyloxy)carbonyl)amino)-2-oxopyrrolidin-1-yl)-1,4-dioxaspiro[4.5]decane-7-carboxylic acid (100 g, 239 mmol) in toluene (187 mL) under an atmosphere of nitrogen at rt was added triethylamine (24.18 g, 239 mmol). The mixture was cooled to −10° C. and isobutyl carbonochloridate (32.6 g, 239 mmol) was added. The mixture was stirred at 0° C. to 10° C. for 30 min, a solution of sodium azide (28.0 g, 430 mmol) and tetrabutylammonium bromide (3.85 g, 11.95 mmol) in water (310 mL) was added, and the mixture was stirred for at 0° C. to 10° C. for 2 h. Water (230 mL) and toluene (935 mL) were added and the mixture was stirred for 10 min. The organic phase was dried over molecular sieves (4 Å) (50 g). Acetic anhydride (53.7 g, 526 mmol) and acetic acid (14.35 g, 239 mmol) were added and the mixture was stirred at 90° C. for 4 h. The mixture was cooled to rt and was concentrated under reduced pressure. The residue was triturated with n-hexane (2×250 mL) and was dried under reduced pressure to provide the title compound as a pale-yellow gum (80 g, 132 mmol, 55.4% yield). LC-MS m/z 432.1 (M+H)⁺.

### Step 13: Benzyl ((S)-1-((1S,2R)-2-acetamido-4-oxocyclohexyl)-2-oxopyrrolidin-3-yl)carbamate

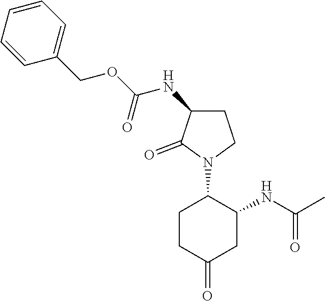 [SMILES: CC(=O)N[C@@H]1CC(=O)CC[C@@H]1N1CC[C@H](NC(=O)OCc2ccccc2)C1=O]

To a stirred solution of benzyl ((S)-1-((7R,8S)-7-acetamido-1,4-dioxaspiro[4.5]decan-8-yl)-2-oxopyrrolidin-3-yl)carbamate (157 g, 364 mmol) in acetone (1200 mL) under an atmosphere of nitrogen at rt was added dropwise 1.5 N HCl (1213 mL, 1819 mmol) over 30 min. The mixture was stirred at 55° C. for 3 h and was concentrated under reduced pressure to remove the acetone. Dichloromethane (DCM) was added. The organic phase was washed with brine (1000 mL), was dried over Na₂SO₄, and was concentrated under reduced pressure. The residue was triturated with ethyl acetate in hexane (10%) (2×500 mL) to provide the title compound as an off-white solid (121 g, 287 mmol, 79% yield). LC-MS m/z 388.2 (M+H)⁺.

### Step 14: Benzyl ((S)-1-((1S,2R,4R)-2-acetamido-4-(tert-butylamino)cyclohexyl)-2-oxopyrrolidin-3-yl)carbamate

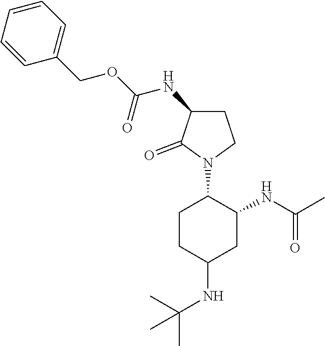 [SMILES: CC(=O)N[C@@H]1CC(NC(C)(C)C)CC[C@@H]1N1CC[C@H](NC(=O)OCc2ccccc2)C1=O]

To a stirred solution of 1 M titanium tetrachloride in dichloromethane (DCM) (96 mL, 96 mmol) in dichloromethane (DCM) (200 mL) under an atmosphere of nitrogen at 0° C. was added dropwise titanium(IV) isopropoxide (28.1 mL, 96 mmol) over 15 min and the mixture was stirred at 0° C. for 10 min. The prepared TiCl2(i-OPr)2 reagent was added dropwise to a stirred solution of benzyl ((S)-1-((1S,2R)-2-acetamido-4-oxocyclohexyl)-2-oxopyrrolidin-3-yl)carbamate (60 g, 155 mmol) and tert-butylamine (85 mL, 802 mmol) in dichloromethane (DCM) (600 mL) at −50° C. over 30 min. The mixture was warmed slowly to rt and was stirred for 2 h. Borane-methyl sulfide complex (15.29 mL, 161 mmol) was added dropwise at 0° C. over 15 min and the mixture was slowly warmed to rt and was stirred for 16 h. Saturated NaHCO₃ (900 mL) and dichloromethane (DCM) (600 mL) were added and the mixture was stirred for 60 min. The slurry was filtered through Celite®, washing with dichloromethane (DCM) (1000 mL). The aqueous phase was extracted with dichloromethane (DCM) (2×500 mL). 1 N HCl (500 mL) was added to the combined organic phases and the mixture was stirred for 10 min. The aqueous phase was washed with dichloromethane (DCM) (500 mL), the pH was adjusted to 8 to 9 by slowly adding ammonium hydroxide solution (30 mL) over 15 min, and the mixture was extracted with dichloromethane (DCM) (2×500 mL) The combined organic extracts were washed with saturated NH₄Cl (3×300 mL), were dried over Na₂SO₄, and were concentrated under reduced pressure. The solid residue was stirred in diethyl ether (50 mL) for 10 min and was collected by filtration to provide the title compound as an off-white solid (26.2 g, 58.5 mmol, 37.8% yield). LC-MS m/z 445.0 (M+H)⁺.

### Step 15: N-((1R,2S)-2-((S)-3-Amino-2-oxopyrrolidin-1-yl)-5-(tert-butylamino) cyclohexyl)acetamide

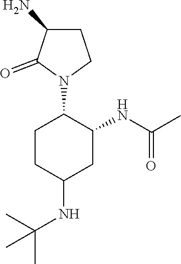 [SMILES: CC(=O)N[C@@H]1CC(NC(C)(C)C)CC[C@@H]1N1CC[C@H](N)C1=O]

A solution of benzyl ((S)-1-((1S,2R,4R)-2-acetamido-4-(tert-butylamino)cyclohexyl)-2-oxopyrrolidin-3-yl)carbamate (8.0 g, 17.99 mmol) in methanol (90 mL) was flushed with nitrogen. 10% Pd—C(1.915 g, 1.799 mmol) was added and the flask was flushed with nitrogen. The mixture was stirred under balloon atmosphere of hydrogen overnight. The mixture was flushed with nitrogen and was filtered through Celite® (2×). The filtrate was concentrated under reduced pressure to provide the title compound as a gray solid (5.5877 g, 18.00 mmol, 100% yield). LC-MS m/z 311.3 (M+H)⁺.

### Intermediate 9—N-((1R,2S,5R)-2-((S)-3-Amino-2-oxopyrrolidin-1-yl)-5-(isopropyl(methyl)amino) cyclohexyl)acetamide

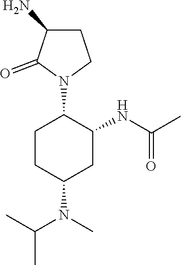 [SMILES: CC(=O)N[C@@H]1C[C@H](N(C)C(C)C)CC[C@@H]1N1CC[C@H](N)C1=O]

### Step 1: Ethyl (1R,2S,5R)-2-((S)-3-(((benzyloxy)carbonyl)amino)-2-oxopyrrolidin-1-yl)-5-(isopropyl(methyl)amino)cyclohexane-1-carboxylate

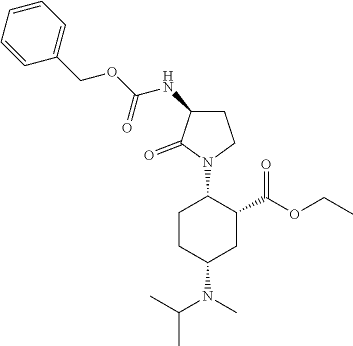 [SMILES: CCOC(=O)[C@@H]1C[C@H](N(C)C(C)C)CC[C@@H]1N1CC[C@H](NC(=O)OCc2ccccc2)C1=O]

To a stirred solution of ethyl (1R,2S)-2-((S)-3-(((benzyloxy)carbonyl)amino)-2-oxopyrrolidin-1-yl)-5-oxocyclohexane-1-carboxylate (Intermediate 8, Step 13) (10 g, 24.85 mmol) in 1,2-dichloroethane (DCE) (160 mL) was added N-methylpropan-2-amine (4.41 mL, 42.2 mmol) and titanium(IV) isopropoxide (10.92 mL, 37.3 mmol) under an atmosphere of nitrogen at rt and the mixture was stirred for 24 h. PtS/C (1.456 g, 24.85 mmol) was added and the mixture was stirred under a balloon atmosphere of hydrogen atmosphere for 48 h. The mixture was filtered through Celite® and the catalyst was washed with dichloromethane (DCM). The filtrate was concentrated under reduced pressure. Water (200 mL) and ethyl acetate (300 mL) were added, the mixture was filtered through Celite®, and the aqueous phase was extracted with ethyl acetate (2×100 mL). The combined organic phases were washed with brine (100 mL), were dried over Na₂SO₄, and were concentrated to provide the title compound as a brown solid (10 g, 20.19 mmol, 81% yield). LC-MS m/z 460.3 (M+H)⁺.

### Step 2: (1R,2S,5R)-2-((S)-3-(((Benzyloxy)carbonyl)amino)-2-oxopyrrolidin-1-yl)-5-(isopropyl(methyl)amino)cyclohexane-1-carboxylic acid

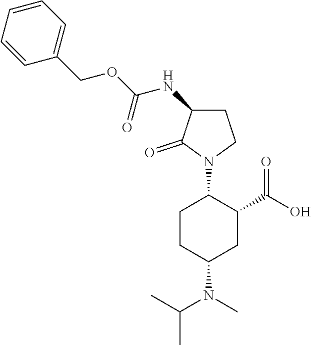 [SMILES: CC(C)N(C)[C@@H]1CC[C@H](N2CC[C@H](NC(=O)OCc3ccccc3)C2=O)[C@H](C(=O)O)C1]

Ethyl (1R,2S,5R)-2-((S)-3-(((benzyloxy)carbonyl)amino)-2-oxopyrrolidin-1-yl)-5-(isopropyl(methyl)amino)cyclohexane-1-carboxylate (10 g, 21.76 mmol) in 15% HCl in water (13.22 mL, 65.3 mmol) was stirred at 45° C. for 24 h. The mixture was cooled to rt, the pH was adjusted to 9 to 10 using 30% wt sodium hydroxide, and the mixture was washed with toluene (3×30 mL). The pH of the aqueous phase was adjusted to 6.5 to 7.5 using 5% wt HCl and the mixture was extracted with dichloromethane (DCM) (2×50 mL). The combined organic extracts were washed with brine (2×30 mL), were dried over Na₂SO₄, were filtered, and the filtrate was concentrated under reduced pressure to obtain the title compound as a light-brown solid (4.4 g, 8.79 mmol, 40.4% yield). LC-MS m/z 432.3 (M+H)⁺.

### Step 3: Benzyl ((S)-1-((1S,2R,4R)-2-((tert-butoxycarbonyl)amino)-4-(isopropyl (methyl)amino)cyclohexyl)-2-oxopyrrolidin-3-yl)carbamate

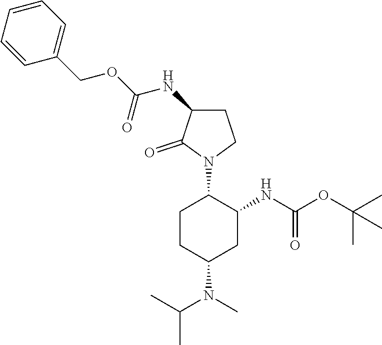 [SMILES: CC(C)N(C)[C@@H]1CC[C@H](N2CC[C@H](NC(=O)OCc3ccccc3)C2=O)[C@H](NC(=O)OC(C)(C)C)C1]

To a mixture of (1R,2S,5R)-2-((S)-3-(((benzyloxy)carbonyl)amino)-2-oxopyrrolidin-1-yl)-5-(isopropyl(methyl)amino)cyclohexanecarboxylic acid (1.6 g, 3.71 mmol) in tert-butanol (25 mL) was added triethylamine (1.793 mL, 12.87 mmol), followed by diphenyl phosphorazidate (0.879 mL, 4.08 mmol) and the mixture was stirred at 80° C. for 16 h. The tert-butanol was removed under reduced pressure and saturated Na₂CO₃ was added. The mixture was stirred for 10 min and was extracted with ethyl acetate (2×35 mL). The combined organic extracts were washed with water (3×30 mL), with brine, were dried over Na₂SO₄, were filtered, and the filtrate was concentrated. Purification by normal-phase chromatography (silica column, 100 to 200 mesh) eluting with a gradient of 3 to 4% methanol in dichloromethane (DCM) provided the title compound as an off-white solid (800 mg, 1.582 mmol, 42.7% yield). LC-MS m/z 503.4 (M+H)⁺.

### Step 4: Benzyl ((S)-1-((1S,2R,4R)-2-amino-4-(isopropyl(methyl)amino)cyclohexyl)-2-oxopyrrolidin-3-yl)carbamate, 2Hydrochloric acid salt

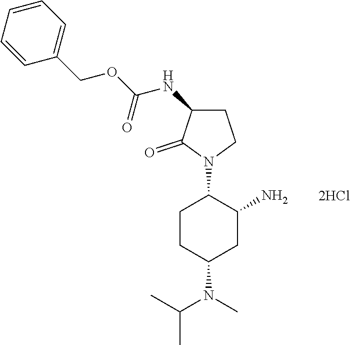 [SMILES: CC(C)N(C)[C@@H]1CC[C@H](N2CC[C@H](NC(=O)OCc3ccccc3)C2=O)[C@H](N)C1]

A mixture of 4 M HCl in 1, 4 dioxane (7.96 mL, 31.8 mmol) and benzyl ((S)-1-((1S,2R,4R)-2-((tert-butoxycarbonyl)amino)-4-(isopropyl(methyl)amino)cyclohexyl)-2-oxopyrrolidin-3-yl)carbamat (800 mg, 1.592 mmol) in 1,4-dioxane (7 mL) was stirred at rt for 3 h. The mixture was concentrated under reduced pressure to provide the title compound as an off-white solid (650 mg, 1.367 mmol, 86% yield). LC-MS m/z 403.2 (M+H)⁺.

### Step 5: Benzyl ((S)-1-((1S,2R,4R)-2-acetamido-4-(isopropyl(methyl)amino) cyclohexyl)-2-oxopyrrolidin-3-yl)carbamate

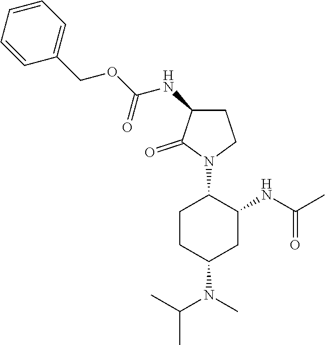 [SMILES: CC(=O)N[C@@H]1C[C@H](N(C)C(C)C)CC[C@@H]1N1CC[C@H](NC(=O)OCc2ccccc2)C1=O]

To a solution of benzyl ((S)-1-((1S,2R,4R)-2-amino-4-(isopropyl(methyl)amino) cyclohexyl)-2-oxopyrrolidin-3-yl)carbamate, 2hydrochloric acid salt (650 mg, 1.367 mmol) in dichloromethane (DCM) (15 mL) was added triethylamine (1.032 mL, 7.40 mmol) and acetic anhydride (0.210 mL, 2.221 mmol) at 0° C. The mixture was stirred at rt for 16 h and was concentrated under reduced pressure. Water (15 mL) was added and the mixture was extracted with methyl t-butyl ether (3×15 mL). The combined organic extracts were dried over anhydrous Na₂SO₄, were filtered, and the filtrate was concentrated under reduced pressure to provide the title compound as a gummy, brown solid (500 mg, 1.120 mmol, 81.9% yield). LC-MS m/z 445.5 (M+H)⁺.

### Step 6: N-((1R,2S,5R)-2-((S)-3-Amino-2-oxopyrrolidin-1-yl)-5-(isopropyl(methyl)amino) cyclohexyl)acetamide

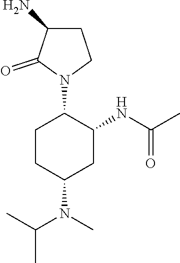 [SMILES: CC(=O)N[C@@H]1C[C@H](N(C)C(C)C)CC[C@@H]1N1CC[C@H](N)C1=O]

To a stirred solution of benzyl ((S)-1-((1S,2R,4R)-2-acetamido-4-(isopropyl(methyl)amino)cyclohexyl)-2-oxopyrrolidin-3-yl)carbamate (500 mg, 1.125 mmol) in ethyl acetate (5 mL) at rt was added 10% Pd—C(1197 mg, 1.125 mmol). The mixture was stirred under a balloon atmosphere of hydrogen at rt for 6 h. Ethyl acetate (20 mL) was added and the mixture was filtered through Celite®. The catalyst was washed with ethyl acetate (20 mL) and the combined filtrates were concentrated to provide the title compound as an off-white, sticky solid (300 mg, 0.948 mmol, 84% yield). LC-MS m/z 311.3 (M+H)⁺.

### Intermediate 10—N-((1R,2S,5R)-5-(tert-Butylamino)-2-((S)-3-((2-chloro-6-(trifluoromethyl) quinazolin-4-yl)amino)-2-oxopyrrolidin-1-yl)cyclohexyl)acetamide

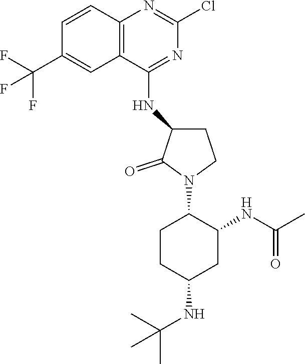 [SMILES: CC(=O)N[C@@H]1C[C@H](NC(C)(C)C)CC[C@@H]1N1CC[C@H](Nc2nc(Cl)nc3ccc(C(F)(F)F)cc23)C1=O]

To a stirred solution of N-((1R,2S,5R)-2-((S)-3-amino-2-oxopyrrolidin-1-yl)-5-(tert-butylamino)cyclohexyl)acetamide (Intermediate 8) (3.5 g, 11.27 mmol) under an atmosphere of nitrogen in dichloromethane (DCM) (50 mL) was added DIPEA (5.91 mL, 33.8 mmol), followed by 2,4-dichloro-6-(trifluoromethyl)quinazoline (3.01 g, 11.27 mmol) (Intermediate 1) and the mixture was stirred at rt for 16 h. Water (100 mL) was added and the mixture was extracted with dichloromethane (DCM) (3×50 mL). The combined organic extracts were dried over Na₂SO₄, were filtered, and the filtrate was concentrated. Purification by normal-phase chromatography (silica column, 40 g) eluting with 1% methanol in dichloromethane (DCM) provided the title compound as an off-white solid (2.8 g, 5.08 mmol, 45.0% yield). LC-MS m/z 541.2 (M+H)⁺.

The following intermediates were or could be prepared using procedures analogous to those described for Intermediate 10:

| | | | LC-MS |
|---|---|---|---|
| Intermediate | Compound | Structure | m/z |
| 11 | N-((1R,2S,5R)-2-((S)-3- ((2-Chloro-6-(trifluoro- methyl)quinazolin-4-yl)- amino)-2-oxopyrrolidin- 1-yl)-5-(isopropyl(meth- yl)amino)cyclohexyl)- acetamide | 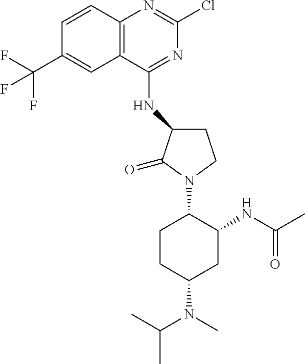 | 311.3 (M + H)⁺ |

### Intermediate 12—N-((1R,2S,5R)-2-((S)-3-((2-((1s,4R)-4-Aminocyclohexyl)-6-(trifluoromethyl)quinazolin-4-yl)amino)-2-oxopyrrolidin-1-yl)-5-(tert-butylamino) cyclohexyl)acetamide, 2Hydrochloric acid salt

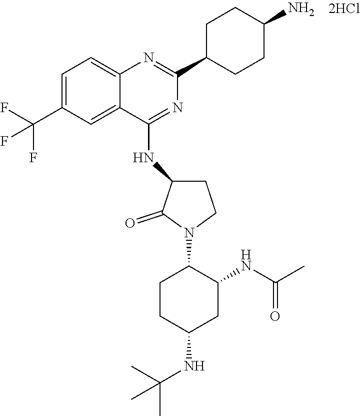 [SMILES: CC(=O)N[C@@H]1C[C@H](NC(C)(C)C)CC[C@@H]1N1CC[C@H](Nc2nc([C@H]3CC[C@@H](N)CC3)nc3ccc(C(F)(F)F)cc23)C1=O]

### Step 1: tert-Butyl (4-(4-(((S)-1-((1S,2R,4R)-2-acetamido-4-(tert-butylamino) cyclohexyl)-2-oxopyrrolidin-3-yl)amino)-6-(trifluoromethyl)quinazolin-2-yl)cyclohex-3-en-1-yl)carbamate

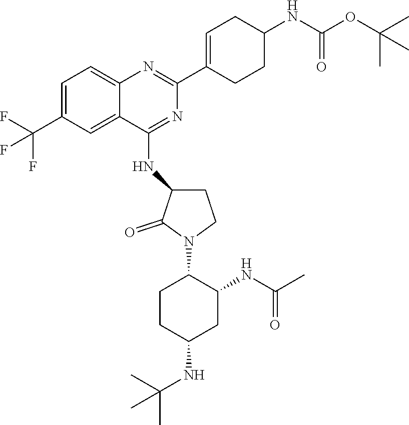 [SMILES: CC(=O)N[C@@H]1C[C@H](NC(C)(C)C)CC[C@@H]1N1CC[C@H](Nc2nc(C3=CCC(NC(=O)OC(C)(C)C)CC3)nc3ccc(C(F)(F)F)cc23)C1=O]

A mixture of N-((1R,2S,5R)-5-(tert-butylamino)-2-((S)-3-((2-chloro-6-(trifluoromethyl) quinazolin-4-yl)amino)-2-oxopyrrolidin-1-yl)cyclohexyl)acetamide (Intermediate 10) (2 g, 3.70 mmol), tert-butyl (4-(4,4,5,5-tetramethyl-1,3,2-dioxaborolan-2-yl)cyclohex-3-en-1-yl)carbamate (1.792 g, 5.55 mmol), P(t-Bu)₃ Pd G4 (0.216 g, 0.370 mmol) and potassium phosphate (1.569 g, 7.39 mmol) in tert-butanol (13.86 mL) and water (4.62 mL) was degassed for 1 h. The mixture was stirred at 80° C. under a balloon atmosphere of nitrogen for 3 h. The mixture was partitioned between dichloromethane (DCM) and water and the aqueous phase was extracted with dichloromethane (DCM). The combined organic phases were dried over Na₂SO₄, were filtered, and the filtrate was concentrated. Purification by ISCO CombiFlash® chromatography (80 g RediSep Rf Gold® column, 60 mL/min) eluting with a gradient of 0 to 15% methanol containing ammonium hydroxide (10%) in dichloromethane (DCM) provided the title compound (2.6 g, 3.70 mmol, 100% yield). LC-MS m/z 702.5 (M+H)⁺.

### Step 2: tert-Butyl ((1R,4s)-4-(4-(((S)-1-((1S,2R,4R)-2-acetamido-4-(tert-butylamino) cyclohexyl)-2-oxopyrrolidin-3-yl)amino)-6-(trifluoromethyl)quinazolin-2-yl)cyclohexyl) carbamate

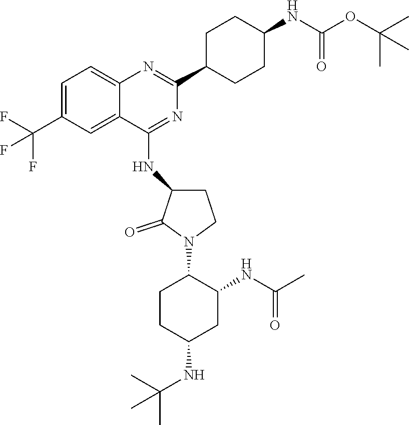 [SMILES: CC(=O)N[C@@H]1C[C@H](NC(C)(C)C)CC[C@@H]1N1CC[C@H](Nc2nc([C@H]3CC[C@@H](NC(=O)OC(C)(C)C)CC3)nc3ccc(C(F)(F)F)cc23)C1=O]

A mixture of tert-butyl (4-(4-(((S)-1-((1S,2R,4R)-2-acetamido-4-(tert-butylamino) cyclohexyl)-2-oxopyrrolidin-3-yl)amino)-6-(trifluoromethyl)quinazolin-2-yl)cyclohex-3-en-1-yl)carbamate (1.56 g, 2.223 mmol) and Pd—C(0.237 g, 0.222 mmol) in methanol (20 mL) and ethyl acetate (10.00 mL) was stirred under a balloon atmosphere of hydrogen at rt overnight. Pd—C (0.024 g, 0.222 mmol) was added and the mixture was stirred under a balloon atmosphere of hydrogen at rt overnight. The catalyst was removed by filtration and was washed with methanol. The filtrate was concentrated and the residue was dissolved in acetonitrile (23 mL) and methanol (3 mL) and was purified by Agilent chiral HPLC (ChromegaChiral™ CC4, 30×250 mm, 5 μm column, 45 mL/min, 2 mL injections) eluting with 2% methanol in acetonitrile to provide the title compound as a white solid (711 mg, 1.010 mmol, 45.4% yield). LC-MS m/z 704.5 (M+H)⁺.

### Step 3: N-((1R,2S,5R)-2-((S)-3-((2-((1s,4R)-4-Aminocyclohexyl)-6-(trifluoromethyl) quinazolin-4-yl)amino)-2-oxopyrrolidin-1-yl)-5-(tert-butylamino)cyclohexyl)acetamide, 2Hydrochloric acid salt

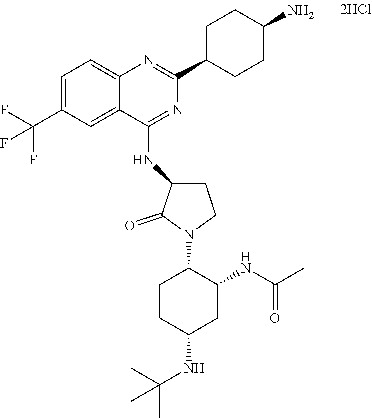 [SMILES: CC(=O)N[C@@H]1C[C@H](NC(C)(C)C)CC[C@@H]1N1CC[C@H](Nc2nc([C@H]3CC[C@@H](N)CC3)nc3ccc(C(F)(F)F)cc23)C1=O]

A mixture of 4 M HCl in 1,4-dioxane (1907 μL, 7.63 mmol) and tert-butyl ((1R,4s)-4-(4-(((S)-1-((1S,2R,4R)-2-acetamido-4-(tert-butylamino)cyclohexyl)-2-oxopyrrolidin-3-yl)amino)-6-(trifluoromethyl)quinazolin-2-yl)cyclohexyl)carbamate (537 mg, 0.763 mmol) was stirred at rt for 1.5 h. The mixture was concentrated to dryness to provide the title compound as a white solid (525 mg, 0.776 mmol, 102% yield). LC-MS m/z 604.4 (M+H)⁺.

The following intermediates were or could be prepared using procedures analogous to those described for Intermediate 12:

| | | | LC-MS |
|---|---|---|---|
| Intermediate | Compound | Structure | m/z |
| 13 | N-((1R,2S,5R)-2-((S)- 3-((2-((1r,4S)-4- Aminocyclohexyl)-6- (trifluoromethyl)quin- azolin-4-yl)amino)-2- oxopyrrolidin-1-yl)-5- (tert-butylamino)- cyclohexyl)acetamide, 2Hydrochloric acid salt | 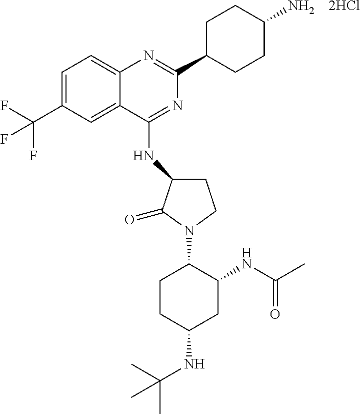 [SMILES: CC(=O)N[C@@H]1C[C@H](NC(C)(C)C)CC[C@@H]1N1CC[C@H](Nc2nc([C@H]3CC[C@H](N)CC3)nc3ccc(C(F)(F)F)cc23)C1=O ; "2HCl"] | 604.4 (M + H)⁺ |

### Intermediate 14—N-((1R,2S,5R)-5-(tert-Butylamino)-2-((S)-2-oxo-3-((2-(piperidin-4-yl)-6-(trifluoromethyl)quinazolin-4-yl)amino)pyrrolidin-1-yl)cyclohexyl)acetamide, 2Hydrochloric acid salt

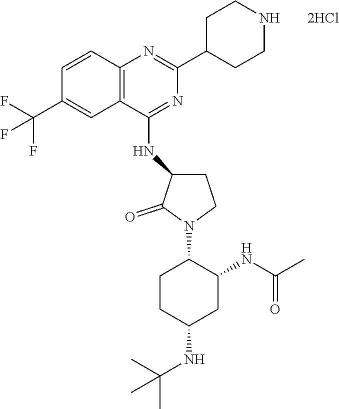 [SMILES: CC(=O)N[C@@H]1C[C@H](NC(C)(C)C)CC[C@@H]1N1CC[C@H](Nc2nc(C3CCNCC3)nc3ccc(C(F)(F)F)cc23)C1=O]

### Step 1: tert-Butyl 4-(4-(((S)-1-((1S,2R,4R)-2-acetamido-4-(tert-butylamino) cyclohexyl)-2-oxopyrrolidin-3-yl)amino)-6-(trifluoromethyl)quinazolin-2-yl)-3,6-dihydropyridine-1(2H)-carboxylate

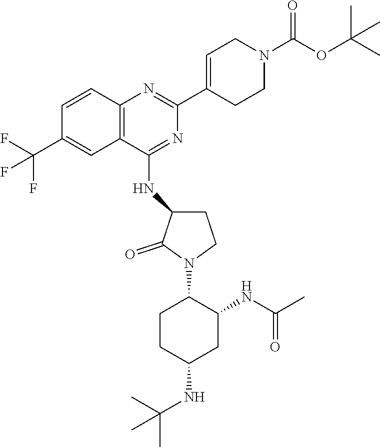 [SMILES: CC(=O)N[C@@H]1C[C@H](NC(C)(C)C)CC[C@@H]1N1CC[C@H](Nc2nc(C3=CCN(C(=O)OC(C)(C)C)CC3)nc3ccc(C(F)(F)F)cc23)C1=O]

N-((1R,2S,5R)-5-(tert-butylamino)-2-((S)-3-((2-chloro-6-(trifluoromethyl)quinazolin-4-yl)amino)-2-oxopyrrolidin-1-yl)cyclohexyl)acetamide (Intermediate 10) (508 mg, 0.939 mmol), tert-butyl 4-(4,4,5,5-tetramethyl-1,3,2-dioxaborolan-2-yl)-3,6-dihydropyridine-1(2H)-carboxylate (436 mg, 1.408 mmol), potassium phosphate (399 mg, 1.878 mmol) and P(t-Bu)₃ Pd Gen 4 (54.9 mg, 0.094 mmol) were placed in tert-butanol (3521 μL) and water (1174 μL) which was degassed. The mixture was stirred at 80° C. for 4 h. The mixture was partitioned between dichloromethane (DCM) and water, and the aqueous phase was extracted with dichloromethane (DCM). The combined organic phases were dried over Na₂SO₄, were filtered, and the filtrate was concentrated. Purification by ISCO CombiFlash® chromatography (40 g RediSep Rf Gold® column, 35 mL/min) eluting with a gradient of 0 to 15% methanol containing ammonium hydroxide (10%) in dichloromethane (DCM) provided the title compound (570 mg, 0.829 mmol, 88% yield). LC-MS m/z 688.4 (M+H)⁺.

### Step 2: tert-Butyl 4-(4-(((S)-1-((1S,2R,4R)-2-acetamido-4-(tert-butylamino) cyclohexyl)-2-oxopyrrolidin-3-yl)amino)-6-(trifluoromethyl)quinazolin-2-yl)piperidine-1-carboxylate

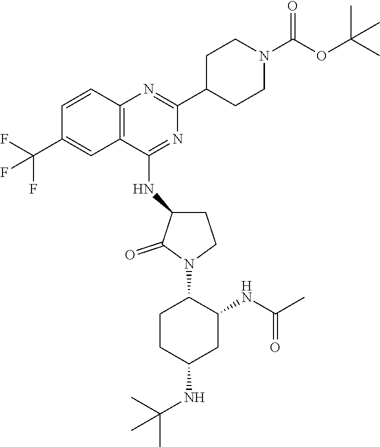 [SMILES: CC(=O)N[C@@H]1C[C@H](NC(C)(C)C)CC[C@@H]1N1CC[C@H](Nc2nc(C3CCN(C(=O)OC(C)(C)C)CC3)nc3ccc(C(F)(F)F)cc23)C1=O]

A mixture of tert-butyl 4-(4-(((S)-1-((1S,2R,4R)-2-acetamido-4-(tert-butylamino) cyclohexyl)-2-oxopyrrolidin-3-yl)amino)-6-(trifluoromethyl)quinazolin-2-yl)-3,6-dihydropyridine-1(2H)-carboxylate (430 mg, 0.625 mmol) and Pd—C(66.5 mg, 0.063 mmol) in methanol (5 mL) was stirred under a balloon at rt under hydrogen balloon overnight. The catalyst was removed by filtration and was washed with methanol. The filtrate was concentrated to provide the title compound (390 mg, 0.565 mmol, 90% yield). LC-MS m/z 690.4 (M+H)⁺.

### Step 3: N-((1R,2S,5R)-5-(tert-Butylamino)-2-((S)-2-oxo-3-((2-(piperidin-4-yl)-6-(trifluoromethyl)quinazolin-4-yl)amino)pyrrolidin-1-yl)cyclohexyl)acetamide, 2 Hydrochloric acid salt

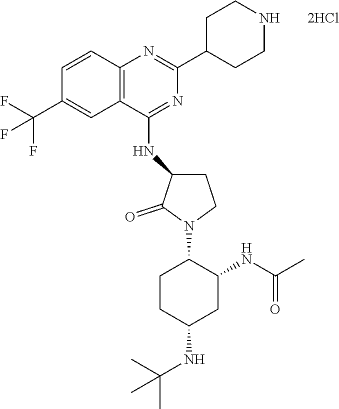 [SMILES: CC(=O)N[C@@H]1C[C@H](NC(C)(C)C)CC[C@@H]1N1CC[C@H](Nc2nc(C3CCNCC3)nc3ccc(C(F)(F)F)cc23)C1=O]

A mixture of 4 N HCl in 1,4-dioxane (1413 μL, 5.65 mmol) and tert-butyl 4-(4-(((S)-1-((1S,2R,4R)-2-acetamido-4-(tert-butylamino)cyclohexyl)-2-oxopyrrolidin-3-yl)amino)-6-(trifluoromethyl)quinazolin-2-yl)piperidine-1-carboxylate (390 mg, 0.565 mmol) was stirred at rt for 2.5 h and was concentrated to dryness to provide the title compound as a white solid (420 mg, 0.634 mmol, 112% yield). LC-MS m/z 590.4 (M+H)⁺.

### Intermediate 15—N-((1R,2S,5R)-5-(tert-Butylamino)-2-((S)-2-oxo-3-((2-(piperidin-4-ylamino)-6-(trifluoromethyl)quinazolin-4-yl)amino)pyrrolidin-1-yl)cyclohexyl)acetamide, 2Hydrochloric acid salt

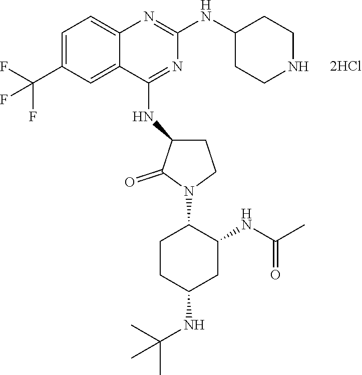 [SMILES: CC(=O)N[C@@H]1C[C@H](NC(C)(C)C)CC[C@@H]1N1CC[C@H](Nc2nc(NC3CCNCC3)nc3ccc(C(F)(F)F)cc23)C1=O]

### Step 1: tert-butyl 4-((4-(((S)-1-((1S,2R,4R)-2-acetamido-4-(tert-butylamino) cyclohexyl)-2-oxopyrrolidin-3-yl)amino)-6-(trifluoromethyl)quinazolin-2-yl)amino)piperidine-1-carboxylate

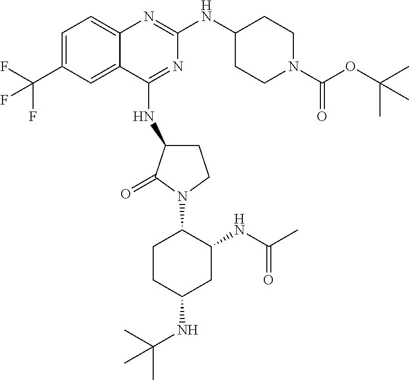 [SMILES: CC(=O)N[C@@H]1C[C@H](NC(C)(C)C)CC[C@@H]1N1CC[C@H](Nc2nc(NC3CCN(C(=O)OC(C)(C)C)CC3)nc3ccc(C(F)(F)F)cc23)C1=O]

N-((1R,2S,5R)-5-(tert-butylamino)-2-((S)-3-((2-chloro-6-(trifluoromethyl)quinazolin-4-yl)amino)-2-oxopyrrolidin-1-yl)cyclohexyl)acetamide (Intermediate 10) (250 mg, 0.462 mmol), DIEA (0.323 mL, 1.848 mmol) and tert-butyl 4-aminopiperidine-1-carboxylate (370 mg, 1.848 mmol) were dissolved in Ethanol (5 mL). The mixture was heated in a microwave vial to 125° C. for 18 h. The reaction was cooled and LCMS indicated ˜80% product. The ethanol was evaporated. The residue was dissolved in EtOAc and was washed with NaHCO₃ sat. Aq. The organic was dried with sodium sulfate then was filtered and evaporated to give tert-butyl 4-((4-(((S)-1-((1S,2R,4R)-2-acetamido-4-(tert-butylamino)cyclohexyl)-2-oxopyrrolidin-3-yl)amino)-6-(trifluoromethyl)quinazolin-2-yl)amino)piperidine-1-carboxylate (700 mg, 0.497 mmol, minimum purity: 50%). The crude product was carried over to the next step without purification. LC-MS m/z 705.4 (M+H)⁺.

### Step 2: N-((1R,2S,5R)-5-(tert-butylamino)-2-((S)-2-oxo-3-((2-(piperidin-4-ylamino)-6-(trifluoromethyl)quinazolin-4-yl)amino)pyrrolidin-1-yl)cyclohexyl)acetamide, 2Hydrochloride

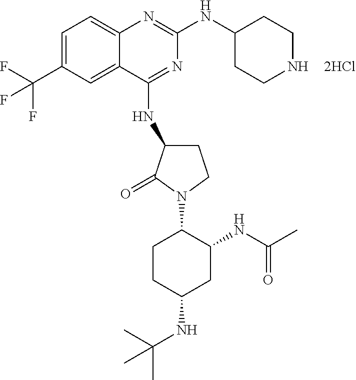 [SMILES: CC(=O)N[C@@H]1C[C@H](NC(C)(C)C)CC[C@@H]1N1CC[C@H](Nc2nc(NC3CCNCC3)nc3ccc(C(F)(F)F)cc23)C1=O ; "2HCl"]

The crude tert-butyl 4-((4-(((S)-1-((1S,2R,4R)-2-acetamido-4-(tert-butylamino) cyclohexyl)-2-oxopyrrolidin-3-yl)amino)-6-(trifluoromethyl)quinazolin-2-yl)amino)piperidine-1-carboxylate (700 mg, 0.497 mmol, minimum purity: 50%) in DCM was treated with 4M HCl (0.462 mL, 1.848 mmol) in dioxane and stirred at rt for 18 h, no reaction. The reaction mixture was evaporated and the residue was suspended in 15 mL 4 M HCl in dioxane. The reaction was heated to 45° C. for 2 h. LCMS m/z 605.4 (M+H)⁺ showed that the rxn was complete. The reaction mixture was cooled to rt and evaporated to give 720 mg crude product as an off white solid. The product was first purified with MDAP (TFA method), gave an oil (385 mg). LCMS looks OK, >90% clean, but not clean by NMR. It was assumed a mixture of TFA and HCl salts. It was re-purified by MDAP extended run (TFA method, material was dissolved in water). Desired fractions were combined and evaporated, and then evaporated from 1 mL 0.5 N HCl aq to provide N-((1R,2S,5R)-5-(tert-butylamino)-2-((S)-2-oxo-3-((2-(piperidin-4-ylamino)-6-(trifluoromethyl)quinazolin-4-yl)amino)pyrrolidin-1-yl)cyclohexyl)acetamide, 2Hydrochloride (280 mg, 0.393 mmol, 85% yield) as a white foam solid. LCMS m/z 605.4 (M+H)⁺.

The following intermediates were or could be prepared using procedures analogous to those described for Intermediate 15:

| | | | LC-MS |
|---|---|---|---|
| Intermediate | Compound | Structure | m/z |
| 16 | N-((1R,2S,5R)-5-(tert- Butylamino)-2-((S)-2- oxo-3-((2-((piperidin- 4-ylmethyl)amino)-6- (trifluoromethyl)quin- azolin-4-yl)amino)- pyrrolidin-1-yl)cyclo- hexyl)acetamide, 2Hydrochloric acid salt | 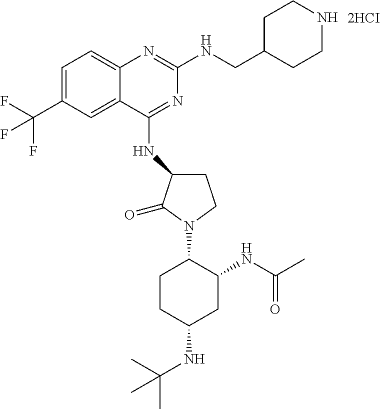 | 619.4 (M + H)⁺ |
| 17 | N-((1R,2S,5R)-2-((S)- 3-((2-((2-aminoethyl)- amino)-6-trifluoro- methyl)quinazolin-4- yl)amino)-2-oxopyr- rolidin-1-yl)-5-(tert- butylamino)cyclohex- yl)acetamide hydro- chloride | 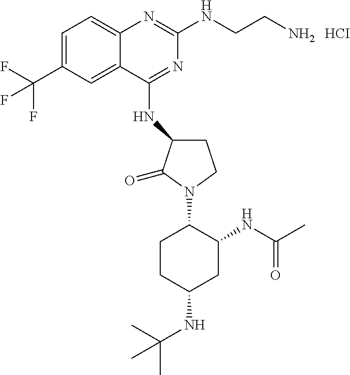 | 565.4 (M + H)⁺ |

### Intermediate 18—N-((1R,2S,5R)-2-((S)-3-((2-(3-Aminopropyl)-6-(trifluoromethyl) quinazolin-4-yl)amino)-2-oxopyrrolidin-1-yl)-5-(tert-butylamino)cyclohexyl)acetamide

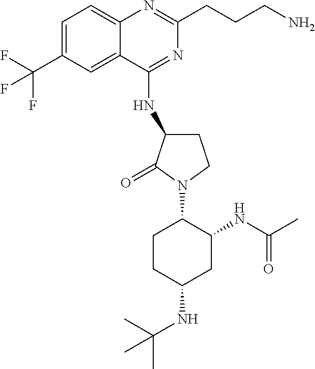 [SMILES: CC(=O)N[C@@H]1C[C@H](NC(C)(C)C)CC[C@@H]1N1CC[C@H](Nc2nc(CCCN)nc3ccc(C(F)(F)F)cc23)C1=O]

### Step 1: N-((1R,2S,5R)-5-(tert-Butylamino)-2-((S)-3-((2-(3-(1,3-dioxoisoindolin-2-yl)propyl)-6-(trifluoromethyl)quinazolin-4-yl)amino)-2-oxopyrrolidin-1-yl)cyclohexyl)acetamide

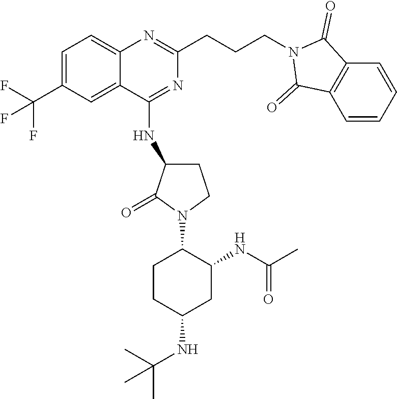 [SMILES: CC(=O)N[C@@H]1C[C@H](NC(C)(C)C)CC[C@@H]1N1CC[C@H](Nc2nc(CCCN3C(=O)c4ccccc4C3=O)nc3ccc(C(F)(F)F)cc23)C1=O]

A suspension of N-((1R,2S,5R)-2-((S)-3-amino-2-oxopyrrolidin-1-yl)-5-(tert-butylamino)cyclohexyl)acetamide (520 mg, 1.675 mmol), 2-(3-(4-chloro-6-(trifluoromethyl)quinazolin-2-yl)propyl)isoindoline-1,3-dione (713 mg, 1.698 mmol) and DIPEA (0.9 mL, 5.15 mmol) in acetonitrile (40 mL) was stirred at rt overnight. N,N-Dimethylformamide (DMF) (3 mL) was added to help with solubility and the mixture was stirred for 5 h. N,N-Dimethylformamide (DMF) (2 mL) and acetonitrile (5 mL) were added, the mixture was stirred overnight and was heated at 65° C. for 2 h. The mixture was concentrated, the residue was partitioned between ethyl acetate (120 mL) and water (75 mL), and the aqueous phase was extracted with ethyl acetate (2×50 mL). The combined organic phases were washed with brine, were dried over MgSO₄, and the filtrate was concentrated under reduced pressure. The residue was purified by SGCC (40 g ISCO gold silica gel column, 40 mL/min flow rate, 0-10% MeOH/DCM for 10CVs and 10% MeOH/DCM for 5CVs) to afford the title compound as a pale yellow solid (496 mg). More product was present in the aqeuous phase. Evaporation to remove water gave the residue that was washed with MeCN, filtered and concentrated. The residue was dissolved in water and MeCN, and purified by MDAP (LPH_MethA-2: 5-35% Acetonitrile in Water both containing 0.1% v/v solution of Formic Acid) to yield more product (179 mg). Both batches were combined to given the title compound (675 mg, 0.924 mmol, 55.2% yield). LC-MS m/z 694.0 (M+H)⁺.

### Step 2: N-((1R,2S,5R)-2-((S)-3-((2-(3-Aminopropyl)-6-(trifluoromethyl)quinazolin-4-yl)amino)-2-oxopyrrolidin-1-yl)-5-(tert-butylamino)cyclohexyl)acetamide

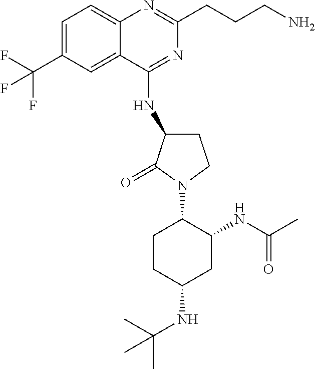 [SMILES: CC(=O)N[C@@H]1C[C@H](NC(C)(C)C)CC[C@@H]1N1CC[C@H](Nc2nc(CCCN)nc3ccc(C(F)(F)F)cc23)C1=O]

To a mixture of N-((1R,2S,5R)-5-(tert-butylamino)-2-((S)-3-((2-(3-(1,3-dioxoisoindolin-2-yl)propyl)-6-(trifluoromethyl)quinazolin-4-yl)amino)-2-oxopyrrolidin-1-yl)cyclohexyl)acetamide (500 mg, 0.721 mmol) in ethanol (5 mL) was added hydrazine monohydrate (0.106 mL, 2.162 mmol). The mixture was stirred at rt overnight and ethanol was removed under reduced pressure. The residue was suspended in dichloromethane (DCM) (30 mL) and sonicated. The solid was removed by filtration and was washed with dichloromethane (DCM). The filtrated was concentrated to provide the title compound as a yellow film (414 mg, 0.676 mmol, 94% yield). LC-MS m/z 564.1 (M+H)⁺.

The following intermediates were or could be prepared using procedures analogous to those described for Intermediate 18:

| | | | LC-MS |
|---|---|---|---|
| Intermediate | Compound | Structure | m/z |
| 19 | N-((1R,2S,5R)-2-((S)- 3-((2-(2-Aminoethyl)- 6-(trifluoromethyl)quin- azolin-4-yl)amino)-2- oxopyrrolidin-1-yl)-5- (tert-butylamino)cyclo- hexyl)acetamide | 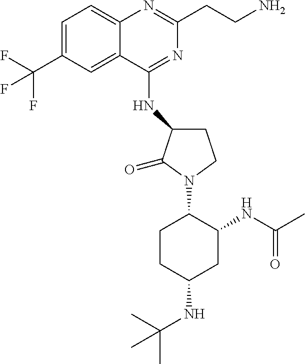 [SMILES: CC(=O)N[C@@H]1C[C@H](NC(C)(C)C)CC[C@@H]1N1CC[C@H](Nc2nc(CCN)nc3ccc(C(F)(F)F)cc23)C1=O] | 550.5 (M + H)⁺ |
| 20 | N-((1R,2S,5R)-2-((S)- 3-((2-(4-aminobutyl)- 6-(trifluoromethyl)quin- azolin-4-yl)amino)-2- oxopyrrolidin-1-yl)-5- (tert-butylamino)cyclo- hexyl)acetamide | 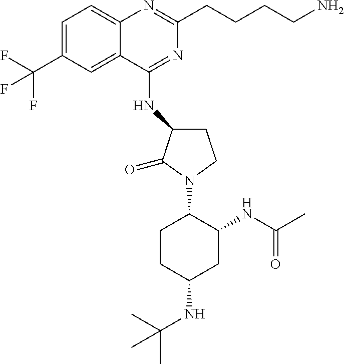 [SMILES: CC(=O)N[C@@H]1C[C@H](NC(C)(C)C)CC[C@@H]1N1CC[C@H](Nc2nc(CCCCN)nc3ccc(C(F)(F)F)cc23)C1=O] | 578.06 (M + H)⁺ |
| 21 | N-((1R,2S,5R)-2-((S)- 3-((2-((2-Aminoeth- oxy)methyl)-6-(tri- fluoromethyl)quinazo- lin-4-yl)amino)-2-oxo- pyrrolidin-1-yl)-5-(tert- butylamino)cyclohex- yl)acetamide | 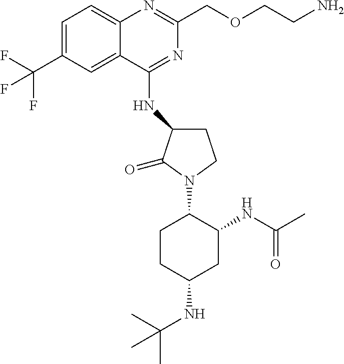 [SMILES: CC(=O)N[C@@H]1C[C@H](NC(C)(C)C)CC[C@@H]1N1CC[C@H](Nc2nc(COCCN)nc3ccc(C(F)(F)F)cc23)C1=O] | 580.5 (M + H)⁺ |
| 22 | N-((1R,2S,5R)-2-((S)- 3-((2-(3-Aminopropyl)- 6-(trifluoromethyl)quin- azolin-4-yl)amino)-2- oxopyrrolidin-1-yl)-5- (isopropyl(methyl)- amino)cyclohexyl)acet- amide | 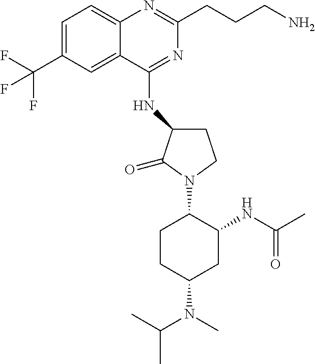 [SMILES: CC(=O)N[C@@H]1C[C@H](N(C)C(C)C)CC[C@@H]1N1CC[C@H](Nc2nc(CCCN)nc3ccc(C(F)(F)F)cc23)C1=O] | 564.2 (M + H)⁺ |

### Intermediate 23—N-((1R,2S,5R)-5-(tert-Butylamino)-2-((S)-2-oxo-3-((2-(piperidin-4-yloxy)-6-(trifluoromethyl)quinazolin-4-yl)amino)pyrrolidin-1-yl)cyclohexyl)acetamide, 2Hydrochloric acid salt

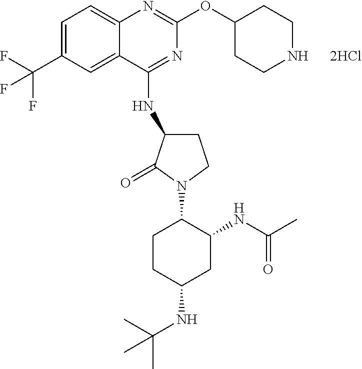 [SMILES: CC(=O)N[C@@H]1C[C@H](NC(C)(C)C)CC[C@@H]1N1CC[C@H](Nc2nc(OC3CCNCC3)nc3ccc(C(F)(F)F)cc23)C1=O ; "2HCl"]

### Step 1: tert-Butyl 4-((4-(((S)-1-((1S,2R,4R)-2-acetamido-4-(tert-butylamino) cyclohexyl)-2-oxopyrrolidin-3-yl)amino)-6-(trifluoromethyl)quinazolin-2-yl)oxy)piperidine-1-carboxylate

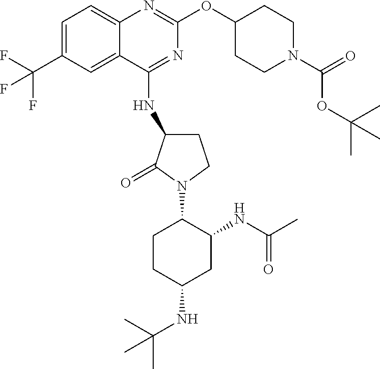 [SMILES: CC(=O)N[C@@H]1C[C@H](NC(C)(C)C)CC[C@@H]1N1CC[C@H](Nc2nc(OC3CCN(C(=O)OC(C)(C)C)CC3)nc3ccc(C(F)(F)F)cc23)C1=O]

To a solution of tert-butyl 4-((4-(butylsulfinyl)-6-(trifluoromethyl)quinazolin-2-yl)oxy)piperidine-1-carboxylate (intermediate 6) (713 mg, 1.422 mmol) in acetonitrile (15 mL) was added DIPEA (0.993 mL, 5.69 mmol) followed by N-((1R,2S,5R)-2-((S)-3-amino-2-oxopyrrolidin-1-yl)-5-(tert-butylamino)cyclohexyl)acetamide (Intermediate 8) (530 mg, 1.706 mmol) and the mixture was stirred at 60° C. for 5 days. The mixture was cooled to rt, ethyl acetate and ISOLUTE® absorbent were added, and the mixture was concentrated under reduced pressure. Purification by normal-phase chromatography (40 g silica) eluting with a stepwise gradient of 0 to 100% ethyl acetate in heptane and then 0 to 100% methanol/dichloromethane (DCM) (20%) in dichloromethane provided the title compound as a clear colorless oil (555.8 mg, 0.787 mmol, 55.4% yield). LC-MS m/z 706.7 (M+H)⁺.

### Step 2: N-((1R,2S,5R)-5-(tert-Butylamino)-2-((S)-2-oxo-3-((2-(piperidin-4-yloxy)-6-(trifluoromethyl)quinazolin-4-yl)amino)pyrrolidin-1-yl)cyclohexyl)acetamide, 2 Hydrochloric acid salt

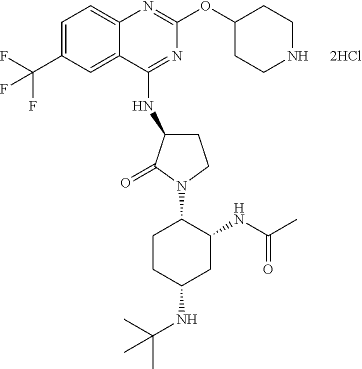 [SMILES: CC(=O)N[C@@H]1C[C@H](NC(C)(C)C)CC[C@@H]1N1CC[C@H](Nc2nc(OC3CCNCC3)nc3ccc(C(F)(F)F)cc23)C1=O ; "2HCl"]

A mixture of 4 M HCl in 1,4-dioxane (0.903 mL, 3.61 mmol) and tert-butyl 4-((4-(((S)-1-((1S,2R,4R)-2-acetamido-4-(tert-butylamino)cyclohexyl)-2-oxopyrrolidin-3-yl)amino)-6-(trifluoromethyl)quinazolin-2-yl)oxy)piperidine-1-carboxylate (509.9 mg, 0.722 mmol) in dichloromethane (DCM) (7.0 mL) was stirred at rt overnight. The mixture was concentrated under reduced pressure to provide the title compound as a white solid (526.8 mg, 0.820 mmol, 114% yield). LC-MS m/z 606.6 (M+H)⁺.

The following intermediates were or could be prepared using procedures analogous to those described for Intermediate 23:

| | | | LC-MS |
|---|---|---|---|
| Intermediate | Compound | Structure | m/z |
| 24 | N-((1R,2S,5R)-5-(tert- Butylamino)-2-((S)-2- oxo-3-((2-(piperidin- 4-ylmethoxy)-6- (trifluoromethyl)quin- azolin-4-yl)amino)pyr- rolidin-1-yl)cyclohex- yl)acetamide, 2Hydro- chloric acid salt | 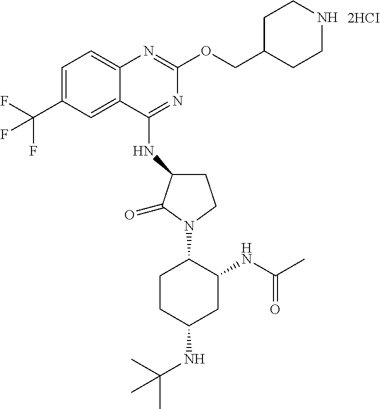 | 620.6 (M + H)⁺ |

### Intermediate 25—(2S,3S)-1-Methyl-5-oxo-2-(pyridin-3-yl)pyrrolidine-3-carboxylic acid

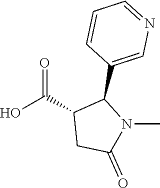 [SMILES: CN1C(=O)C[C@H](C(=O)O)[C@H]1c1cccnc1]

Total of racemic-trans-1-methyl-5-oxo-2-(pyridin-3-yl)pyrrolidine-3-carboxylic acid (500 g, 2.27 mmol) was separated into 50 g batches. 50 g batch of material was dissolved in boiling methanol (750 mL). It was significant insolubles. Acetonitrile (750 mL) was added. It was filtered through glass fiber paper. Same procedure was repeated for the remaining batches. Total 440 g dissolved material and 60 g of insoluble filtered material. The dissolved racemate was separated by Agilent 1100 chiral HPLC (Chiralpak IA 101×210 mm 20 μm column, 500 mL/min, 59×7.5 g injections) eluting with acetonitrile/methanol/formic acid (50:50:0.1) to provide (2S,3S)-1-methyl-5-oxo-2-(pyridin-3-yl)pyrrolidine-3-carboxylic acid as a white solid (192.1 g, 829 mmol, 36.5% yield). LC-MS m/z 221.1 (M+H)⁺. Chiral HPLC: 98% at RT=3.372 min,

The following intermediates were or could be prepared using procedures analogous to those described for Intermediate 25:

| | | | LC-MS |
|---|---|---|---|
| Intermediate | Compound | Structure | m/z |
| 26 | (2S,3S)-1-ethyl-5-oxo-2- (pyridin-3-yl)pyrrolidine- 3-carboxylic acid | 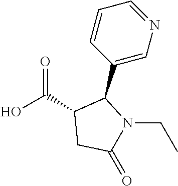 [SMILES: CCN1C(=O)C[C@H](C(=O)O)[C@H]1c1cccnc1] | 235.2 (M + H)⁺ |

### Example 1—(2S,3S)—N-(2-(((1R,4S)-4-(4-(((1S,4R)-4-((3-(4-(((S)-1-((1S,2R,4R)-2-Acetamido-4-(tert-butylamino)cyclohexyl)-2-oxopyrrolidin-3-yl)amino)-6-(trifluoromethyl)quinazolin-2-yl)propyl)carbamoyl)cyclohexyl)amino)-4-oxobutoxy)cyclohexyl)oxy)ethyl)-1-methyl-5-oxo-2-(pyridin-3-yl)pyrrolidine-3-carboxamide

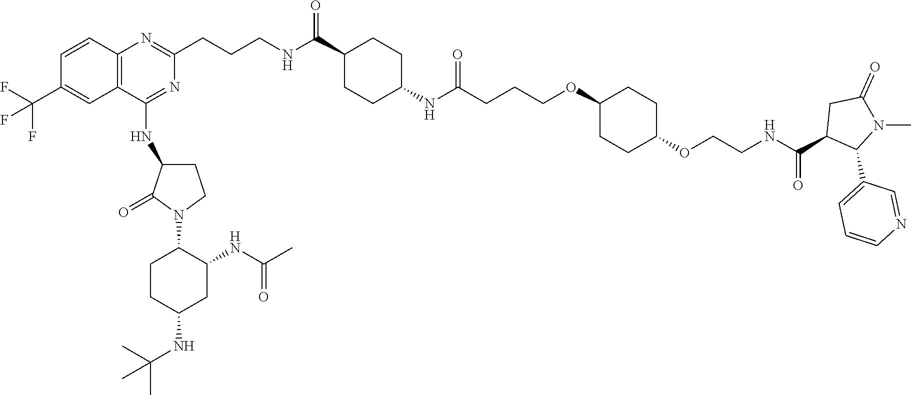 [SMILES: CC(=O)N[C@@H]1C[C@H](NC(C)(C)C)CC[C@@H]1N1CC[C@H](Nc2nc(CCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O]

### Step 1: N,N-Dibenzyl-2-chloroethan-1-amine

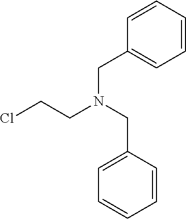 [SMILES: ClCCN(Cc1ccccc1)Cc1ccccc1]

Saturated NaHCO₃ was added to N,N-dibenzyl-2-chloroethan-1-amine, Hydrochloride (21 g, 70.9 mmol) (21 g, 70.9 mmol) at 0° C. and the mixture was extracted with dichloromethane (DCM). The combined organic extracts were dried over Na₂SO₄, were filtered, and the filtrate was concentrated to provide the title compound (18 g, 69.3 mmol, 98% yield). LC-MS m/z 260.3 (M+H)⁺.

### Step 2: (1r,4r)-4-(2-(Dibenzylamino)ethoxy)cyclohexan-1-ol

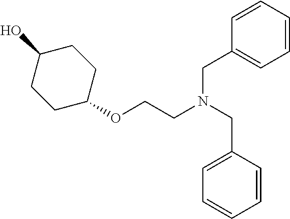 [SMILES: O[C@H]1CC[C@H](OCCN(Cc2ccccc2)Cc2ccccc2)CC1]

To a mixture of (1r,4r)-cyclohexane-1,4-diol (2 g, 17.22 mmol) in N,N-dimethylformamide (DMF) (20 mL) at 0° C. was added 60% sodium hydride (2.4 g, 60.0 mmol) and the mixture was stirred at rt for 30 min. The mixture was cooled to 0° C., N,N-dibenzyl-2-chloroethan-1-amine (5 g, 19.25 mmol) was added, and the mixture was stirred at 80° C. for 2 days. The mixture was cooled to rt, was poured into ice, and was extracted with ethyl acetate. The combined organic extracts were washed with water, with brine, were dried over Na₂SO₄, were filtered, and the filtrate was concentrated. Purification by reverse-phase HPLC (C18 Aq Gold 275 g column, 150 mL/min) eluting with a gradient of 40 to 63% acetonitrile containing ammonium hydroxide (1%) in water containing ammonium bicarbonate (10 mM) and ammonium hydroxide (0.075%) provided the title compound (961 mg, 2.83 mmol, 16.44% yield). LC-MS m/z 340.3 (M+H)⁺.

### Step 3: Methyl (E)-4-(((1r,4r)-4-(2-(dibenzylamino)ethoxy)cyclohexyl)oxy)but-2-enoate

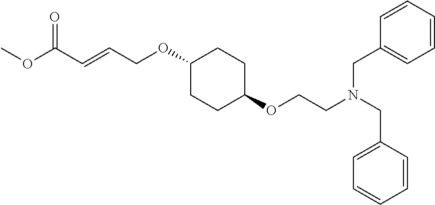 [SMILES: COC(=O)/C=C/CO[C@H]1CC[C@H](OCCN(Cc2ccccc2)Cc2ccccc2)CC1]

A mixture of (1r,4r)-4-(2-(dibenzylamino)ethoxy)cyclohexan-1-ol (8.86 g, 26.1 mmol), methyl but-2-ynoate (5.12 g, 52.2 mmol), triphenylphosphine (0.685 g, 2.61 mmol), and acetic acid (0.598 mL, 10.44 mmol) in dry toluene (100 mL) was heated at reflux with stirring under an atmosphere of nitrogen for 21 h. The mixture was concentrated under reduced pressure. The residue was dissolved in ethyl acetate (200 mL) and was washed with saturated NaCl. The aqueous layer was extracted with ethyl acetate (100 mL). The combined organic phases were washed with saturated NaCl, were dried over Na₂SO₄, were filtered, and the filtrate was concentrated under reduced pressure. Purification by ISCO CombiFlash® chromatography (330 g RediSep Rf Gold® column, 150 mL/min) eluting with a gradient of 0 to 20% ethyl acetate in heptane provided the title compound as an orange oil (8.84 g, 20.20 mmol, 77% yield). LC-MS m/z 438.4 (M+H)⁺. ¹H NMR (400 MHz, DMSO-d₆) δ ppm 7.29-7.42 (m, 8H) 7.19-7.27 (m, 2H) 6.91 (dt, J=15.7, 4.2 Hz, 1H) 6.00 (dt, J=15.7, 2.2 Hz, 1H) 4.15 (dd, J=4.2, 2.2 Hz, 2H) 3.66 (t, J=7.4 Hz, 3H) 3.49 (t, J=6.1 Hz, 2H) 3.33 (s, 3H) 3.19-3.32 (m, 2H) 2.52-2.57 (m, 2H) 1.81-1.91 (m, 4H) 1.15-1.29 (m, 4H). NMR indicated about a 9:1 E:Z mixture.

### Step 4: (E)-4-(((1r,4r)-4-(2-(Dibenzylamino)ethoxy)cyclohexyl)oxy)but-2-enoic acid, Sodium salt

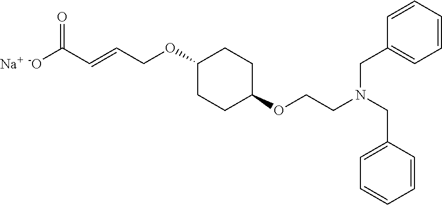

To a solution of methyl (E)-4-(((1,4-trans)-4-(2-(dibenzylamino)ethoxy)cyclohexyl) oxy)but-2-enoate (8.83 g, 20.18 mmol) tetrahydrofuran (THF) (20 mL) was added 5.09 M sodium hydroxide (4.00 mL, 20.38 mmol) and the mixture was heated at reflux for 22 h. 5.09 M Sodium hydroxide (0.793 mL, 4.04 mmol) was added. The mixture was refluxed for 5 h and was concentrated under reduced pressure. The residue was azeotroped with toluene and was placed under high vacuum to provide the title compound as a sticky, pale-yellow solid (8.99 g, 20.13 mmol, 100% yield). LC-MS m/z 424.4 (M+H)⁺.

### Step 5: tert-Butyl (1R,4r)-4-((E)-4-(((1r,4R)-4-(2-(dibenzylamino)ethoxy)cyclohexyl) oxy)but-2-enamido)cyclohexane-1-carboxylate

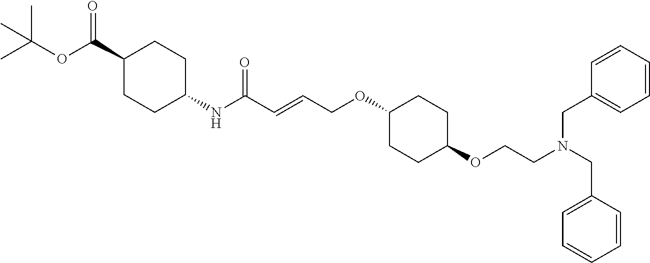 [SMILES: CC(C)(C)OC(=O)[C@H]1CC[C@H](NC(=O)/C=C/CO[C@H]2CC[C@H](OCCN(Cc3ccccc3)Cc3ccccc3)CC2)CC1]

To a stirred suspension of (E)-4-(((1,4-trans)-4-(2-(dibenzylamino)ethoxy) cyclohexyl)oxy)but-2-enoic acid, sodium salt (8.97 g, 20.09 mmol) in dry N,N-dimethylformamide (DMF) (40 mL) were added HATU (8.40 g, 22.10 mmol), tert-butyl (1r,4r)-4-aminocyclohexane-1-carboxylate (4.00 g, 20.09 mmol) in N,N-dimethylformamide (DMF) (10 mL) was added, followed by DIPEA (10.53 mL, 60.3 mmol), and the mixture was stirred at rt for 25 h. Ethyl acetate (200 mL) and water (200 mL) were added and the mixture was stirred for 10 min. The aqueous phase was extracted with ethyl acetate (2×150 mL). The combined organic phases were washed with saturated NaCl, were dried over Na₂SO₄, were filtered, and the filtrate was concentrated in under reduced pressure. Purification by ISCO CombiFlash® chromatography (330 g RediSep Rf Gold® column, 150 mL/min) eluting with a gradient of 0 to 80% ethyl acetate in heptane provided the title compound as a white solid (6.72 g, 11.11 mmol, 55.3% yield). LC-MS m/z 605.5 (M+H)⁺.

### Step 6: tert-Butyl (1R,4r)-4-(4-(((1r,4R)-4-(2-aminoethoxy)cyclohexyl)oxy) butanamido) cyclohexane-1-carboxylate

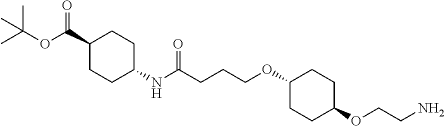 [SMILES: CC(C)(C)OC(=O)[C@H]1CC[C@H](NC(=O)CCCO[C@H]2CC[C@H](OCCN)CC2)CC1]

To a stirred solution of tert-butyl (1R,4r)-4-((E)-4-(((1r,4R)-4-(2-(dibenzylamino)ethoxy)cyclohexyl)oxy)but-2-enamido)cyclohexane-1-carboxylate (6.72 g, 11.11 mmol) in isopropanol (130 mL) was added 10% wet Pd/C (0.672 g, 6.31 mmol) and the flask was evacuated. The mixture was stirred under a balloon atmosphere of hydrogen at rt for 16 h. The mixture was degassed and was filtered through Celite® (2×). The filtrate was concentrated under reduced pressure to provide the title compound as a waxy, gray solid (4.8 g, 11.25 mmol, 101% yield). LC-MS m/z 427.5 (M+H)⁺.

### Step 7: tert-Butyl (1R,4r)-4-(4-(((1S,4R)-4-(2-((2S,3S)-1-methyl-5-oxo-2-(pyridin-3-yl)pyrrolidine-3-carboxamido)ethoxy)cyclohexyl)oxy)butanamido)cyclohexane-1-carboxylate

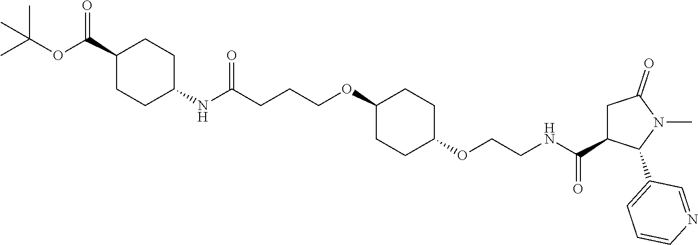 [SMILES: CN1C(=O)C[C@H](C(=O)NCCO[C@H]2CC[C@H](OCCCC(=O)N[C@H]3CC[C@H](C(=O)OC(C)(C)C)CC3)CC2)[C@H]1c1cccnc1]

To a stirred suspension of (2S,3S)-1-methyl-5-oxo-2-(pyridin-3-yl)pyrrolidine-3-carboxylic acid (2.323 g, 10.55 mmol) dichloromethane (DCM) (30 mL) was added HATU (4.41 g, 11.60 mmol) and the mixture was stirred for 15 min. A solution of tert-butyl (1R,4r)-4-(4-(((1r,4R)-4-(2-aminoethoxy)cyclohexyl)oxy)butanamido)cyclohexane-1-carboxylate (4.5 g, 10.55 mmol) in dichloromethane (DCM) (20 mL) was added dropwise over 15 min. A solution of DIPEA (5.53 mL, 31.6 mmol) in dichloromethane (DCM) 120 mL) was added dropwise over 10 min. The mixture was stirred rt for 90 min was concentrated under reduced pressure. The residue was dissolved in dichloromethane (DCM) (100 mL), saturated NaHCO₃ was added, and the mixture was filtered. The organic phase was washed with saturated NaCl, was dried over Na₂SO₄, was filtered, and the filtrate was concentrated under reduced pressure. Purification by ISCO CombiFlash® chromatography (330 g RediSep Rf Gold® column, 150 mL/min) eluting with a gradient of 0 to 10% methanol in dichloromethane (DCM) provided the title compound as a light-pink solid (4.31 g, 6.85 mmol, 65% yield). LC-MS m/z 629.4 (M+H)⁺.

### Step 8: (1R,4r)-4-(4-(((1S,4R)-4-(2-((2S,3S)-1-Methyl-5-oxo-2-(pyridin-3-yl) pyrrolidine-3-carboxamido)ethoxy)cyclohexyl)oxy)butanamido)cyclohexane-1-carboxylic acid, Hydrochloric acid salt

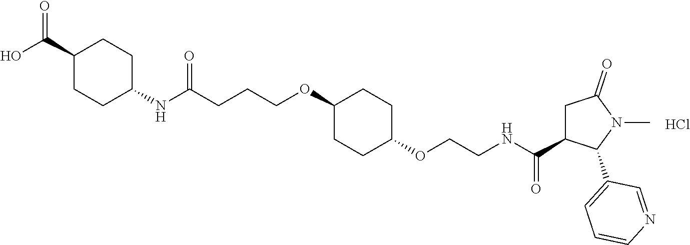 [SMILES: CN1C(=O)C[C@H](C(=O)NCCO[C@H]2CC[C@H](OCCCC(=O)N[C@H]3CC[C@H](C(=O)O)CC3)CC2)[C@H]1c1cccnc1 ; Cl]

To a stirred solution of tert-butyl (1R,4r)-4-(4-(((1S,4R)-4-(2-((2S,3S)-1-methyl-5-oxo-2-(pyridin-3-yl)pyrrolidine-3-carboxamido)ethoxy)cyclohexyl)oxy)butanamido)cyclohexane-1-carboxylate (4.31 g, 6.85 mmol) dry 1,4-dioxane (13 mL) was added 4 M HCl in 1,4-dioxane (38.6 mL, 154 mmol) and the mixture was stirred at rt for 1 h. The mixture was concentrated under reduced pressure at 55° C. and the residue was placed under high vacuum for 15 h to provide the title compound as an off-white solid (4.22 g, 6.93 mmol, 101% yield). LC-MS m/z 573.1 (M+H)⁺.

### Step 9: (2S,3S)—N-(2-(((1R,4S)-4-(4-(((1S,4R)-4-((3-(4-(((S)-1-((1S,2R,4R)-2-Acetamido-4-(tert-butylamino)cyclohexyl)-2-oxopyrrolidin-3-yl)amino)-6-(trifluoromethyl) quinazolin-2-yl)propyl)carbamoyl)cyclohexyl)amino)-4-oxobutoxy)cyclohexyl)oxy)ethyl)-1-methyl-5-oxo-2-(pyridin-3-yl)pyrrolidine-3-carboxamide

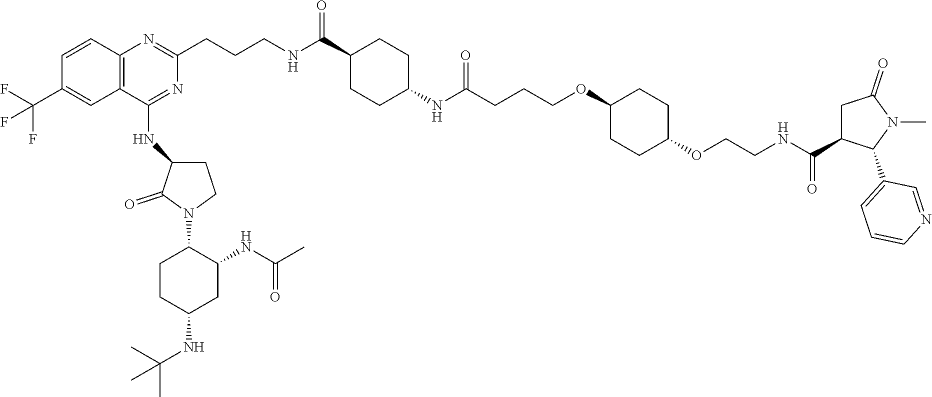 [SMILES: CC(=O)N[C@@H]1C[C@H](NC(C)(C)C)CC[C@@H]1N1CC[C@H](Nc2nc(CCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O]

To a solution of N-((1R,2S,5R)-2-((S)-3-((2-(3-aminopropyl)-6-(trifluoromethyl)quinazolin-4-yl)amino)-2-oxopyrrolidin-1-yl)-5-(tert-butylamino)cyclohexyl) acetamide, 2hydrochloric acid salt (100 mg, 0.157 mmol), (1R,4r)-4-(4-(((1S,4R)-4-(2-((2S,3S)-1-methyl-5-oxo-2-(pyridin-3-yl)pyrrolidine-3-carboxamido)ethoxy)cyclohexyl)oxy)butanamido) cyclohexane-1-carboxylic acid, hydrochloric acid salt (96 mg, 0.157 mmol), HOBt (28.9 mg, 0.189 mmol) and DIPEA (0.137 mL, 0.785 mmol) in dichloromethane (DCM) (2.0 mL) was added EDC (45.2 mg, 0.236 mmol) and the mixture was stirred at rt overnight. Saturated NaHCO₃ was added and the mixture was extracted with dichloromethane (DCM) (3×). The combined organic extracts were washed brine (2×), were dried over Na₂SO₄, were filtered, and the filtrate was concentrated. Purification by MDAP chromatography (XSelect™ CSH Prep 018 5 μm OBD column, 40 mL/min) eluting with a gradient of 30 to 85% acetonitrile in water containing ammonium bicarbonate (10 mM) and ammonium hydroxide (0.075%) provided the title compound as an off-white solid (115.7 mg, 0.098 mmol, 62.6% yield). LC-MS m/z 1118.8 (M+H)(. 1H NMR (400 MHz, METHANOL-d4) δ 8.74 (s, 1H), 8.59-8.54 (m, 1H), 8.50 (d, J=2.0 Hz, 1H), 8.02-7.97 (m, 1H), 7.84-7.76 (1, 2H), 7.55-7.49 (m, 1H), 5.37 (t, J=8.3 Hz, 1H), 4.82 (d, J=6.8 Hz, 1H), 4.56 (br d, J=2.9 Hz, 1° H), 4.03-3.95 (m, 1H), 3.67-3.18 (m, 14H), 3.09-3.00 (m, 1H), 2.88-2.76 (m, 3H), 2.72-2.62 (m, 4H), 2.60-2.49 (m, 1H), 2.34-2.17 (m, 3H), 2.14-1.66 (m, 23H), 1.60 1.44 (i, 2H), 1.34-1.108 (m, (HH).

The following compounds were or could be prepared with procedures analogous to that described in Example 1:

| | | LC-MS | |
|---|---|---|---|
| Ex. | Compound | m/z | NMR |
| 2 | 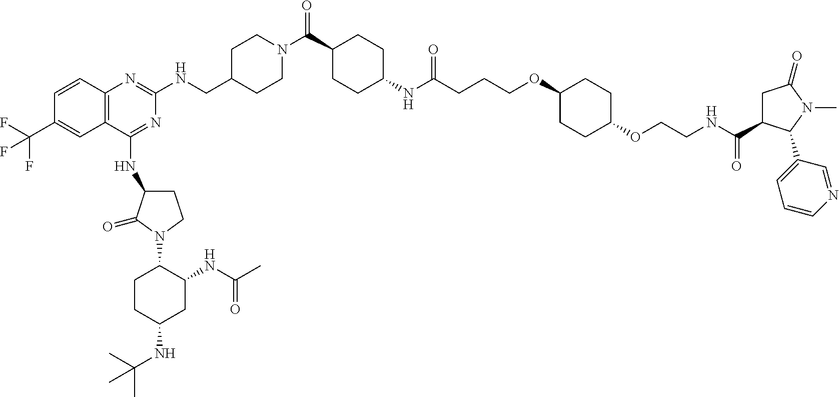 [SMILES: CC(=O)N[C@@H]1C[C@H](NC(C)(C)C)CC[C@@H]1N1CC[C@H](Nc2nc(NCC3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] | 1173.9 (M + H)+ | ¹H NMR (400 MHz, DMSO-d₆) δ ppm 9.73-9.85 (m, 1 H) 8.85-8.71 (m, 1 H) 8.62 (br s, 1 H) 8.56 (dd, J = 4.9, 1.5 Hz, 1 H) 8.47 (d, J = 2.0 Hz, 1 H) 8.22- 8.35 (m, 2 H) 8.04 (br t, J = 5.6 Hz, 1 H) 7.65-7.73 (m, 3 H) 7.44 (dd, J = 7.6, 5.1 Hz, 1 H) 7.37 (br d, J = 8.8 Hz, 1 H) 7.24-7.12 (m, 1 H) 4.91-4.73 (m, 1 H) 4.65 (d, J = 5.9 Hz, 1 H) 4.32-4.43 (m, 2 H) 4.02-4.14 (m, 1 H) 3.84-3.98 (m, 2 H) 3.40-3.52 (m, 3 H) 3.32 (br s, 3 H) 2.91-3.23 (m, 9 H) 2.65-2.74 (m, 1 H) 2.49 (s, 3 H) 2.36-2.46 (m, 2 H) 2.16- 2.33 (m, 2 H) 2.07 (br t, J = 7.3 Hz, 3 H) 1.57-1.94 (m, 22 H) 1.52-1.53 (m, 3 H) 1.13-1.27 (m, 6 H) 1.09 (s, 9 H). |
| | (2S,3S)-N-(2-(((1R,4S)-4-(4-(((1S,4R)-4-(4-(((4-(((S)-1-((1S,2R,4R)-2-Acetamido-4-(tert-butylamino)cyclohexyl)-2-oxopyrrolidin-3- | | |
| | yl)amino)-6-(trifluoromethyl)quinazolin-2-yl)amino)methyl)piperidine-1-carbonyl)cyclohexyl)amino)-4-oxobutoxy)cyclohexyl)- | | |
| | oxy)ethyl)-1-methyl-5-oxo-2-(pyridin-3-yl)pyrrolidine-3-carboxamide. | | |
| 3 | 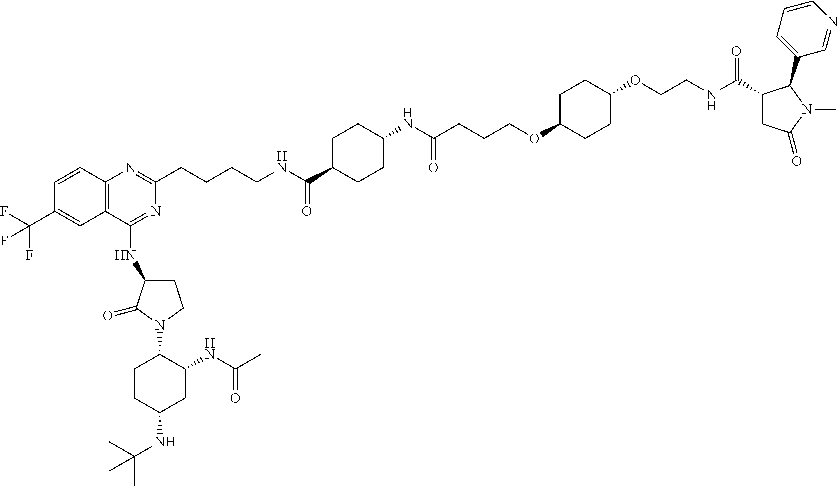 [SMILES: CC(=O)N[C@@H]1C[C@H](NC(C)(C)C)CC[C@@H]1N1CC[C@H](Nc2nc(CCCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] | 1132.7 (M + H)+ | ¹H NMR (400 MHz, DMSO-d₆) δ ppm 9.71 (br s, 1 H) 8.97 (s, 1 H) 8.52-8.68 (m, 2 H) 8.47 (d, J = 2.0 Hz, 1 H) 7.96- 8.08 (m, 2 H) 7.80 (d, J = 8.8 Hz, 1 H) 7.66-7.72 (m, 2 H) 7.64 (d, J = 7.8 Hz, 1 H) 7.44 (dd, J = 7.8, 4.9 Hz, 1 H) 4.96-5.08 (m, 1 H) 4.66 (d, J = 6.4 Hz, 1 H) 4.37-4.46 (m, 1 H) 3.82-3.92 (m, 1 H) 3.37-3.60 (m, 4 H) 3.07 (br s, 9 H) 2.63-2.83 (m, 4 H) 2.49 (s, 3 H) 2.42 (dd, J = 16.6, 7.8 Hz, 1 H) 1.95-2.37 (m, 6 H) 1.53-1.95 (m, 22 H) 1.27-1.52 (m, 4 H) 0.95-1.25 (m, 15 H). |
| | (2S,3S)-N-(2-(((1R,4S)-4-(4-(((1S,4R)-4-((4-(4-(((S)-1-((1S,2R,4R)-2-Acetamido-4-(tert-butylamino)cyclohexyl)-2-oxopyrrolidin-3- | | |
| | yl)amino)-6-(trifluoromethyl)quinazolin-2-yl)butyl)carbamoyl)cyclohexyl)amino)-4-oxobutoxy)cyclohexyl)oxy)ethyl)-1-methyl- | | |
| | 5-oxo-2-(pyridin-3-yl)pyrrolidine-3-carboxamide. | | |
| 4 | 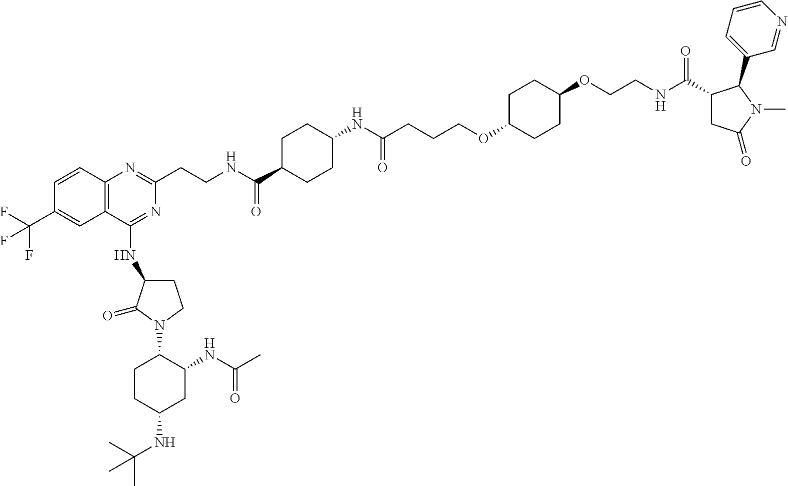 [SMILES: CC(=O)N[C@@H]1C[C@H](NC(C)(C)C)CC[C@@H]1N1CC[C@H](Nc2nc(CCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] | 1104.7 (M + H)+ | ¹H NMR (400 MHz,DMSO-d₆) δ ppm 9.47 (br. s., 1H), 8.96 (s, 1H), 8.66 (d, J = 8.1 Hz, 1H), 8.56 (dd, J = 1.6, 4.8 Hz, 1H), 8.48 (d, J = 2.0 Hz, 1H), 8.24 (s, 1H), 8.10-7.94 (m, 2H), 7.82 (d, J = 8.8 Hz, 1H), 7.74 (t, J = 5.6 Hz, 1H), 7.70-7.59 (m, 2H), 7.44 (dd, J = 4.8, 7.7 Hz, 1H), 5.04 (q, J = 7.7 Hz, 1H), 4.66 (d, J = 6.1 Hz, 1H), 4.37 (br. s., 1H), 3.91 (d, J = 10.3 Hz, 1H), 3.67- 3.26 (m, 17H), 3.25-3.03 (m, 5H), 3.01- 2.91 (m, 1H), 2.86 (t, J = 7.1 Hz, 2H), 2.70 (dd, J = 9.5, 16.6 Hz, 1H), 2.46- 2.26 (m, 1H), 2.13 (br. s., 1H), 2.09- 1.92 (m, 4H), 1.89 (s, 3H), 1.86-1.55 (m, 14H), 1.44-1.26 (m, 2H), 1.22-1.01 (m, 14H) |
| | (2S,3S)-N-(2-(((1R,4S)-4-(4-(((1S,4R)-4-((2-(4-(((S)-1-((1S,2R,4R)-2-Acetamido-4-(tert-butylamino)cyclohexyl)-2-oxopyrrolidin- | | |
| | 3-yl)amino)-6-(trifluoromethyl)quinazolin-2-yl)ethyl)carbamoyl)cyclohexyl)amino)-4-oxobutoxy)cyclohexyl)oxy)ethyl)-1-methyl-5- | | |
| | oxo-2-(pyridin-3-yl)pyrrolidine-3-carboxamide. | | |
| 5 | 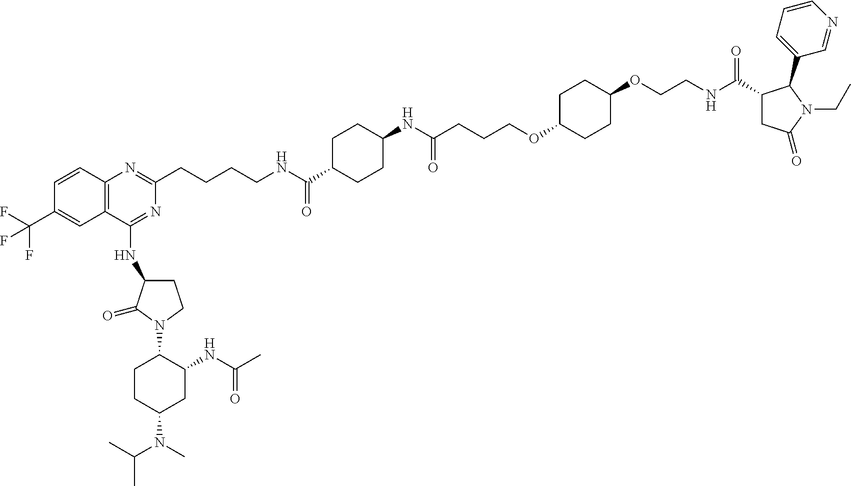 | 1132.0 (M + H)+ | ¹H NMR (400 MHz, DMSO-d₆) δ ppm 9.61-9.83 (m, 1 H) 8.92-9.02 (m, 1 H) 8.60 (d, J = 7.8 Hz, 1 H) 8.56 (dd, J = 4.6, 1.7 Hz, 1 H) 8.52 (d, J = 2.0 Hz, 1 H) 7.96-8.09 (m, 2 H) 7.80 (d, J = 8.8 Hz, 1 H) 7.70-7.77 (m, 2 H) 7.64 (d, J = 7.8 Hz, 1 H) 7.44 (dd, J = 7.8, 4.9 Hz, 1 H) 4.95-5.09 (m, 1 H) 4.75 (d, J = 5.9 Hz, 1 H) 4.42 (br d, J = 3.9 Hz, 1 H) 3.87 (dt, J = 11.9, 3.6 Hz, 1 H) 3.36- 3.60 (m, 5 H) 2.91-3.28 (m, 9 H) 2.62- 2.79 (m, 3 H) 1.97-2.47 (m, 7 H) 1.90 (s, 23 H) 1.09 (s, 18 H) 0.86 (t, J = 7.3 Hz, 3 H). |
| | (2S,3S)-N-(2-(((1R,4S)-4-(4-(((1S,4R)-4-((4-(4-(((S)-1-((1S,2R,4R)-2-Acetamido-4-(tert-butylamino) cyclohexyl)-2-oxopyrrolidin-3- | | |
| | yl)amino)-6-(trifluoromethyl)quinazolin-2-yl)butyl)carbamoyl)cyclohexyl)amino)-4-oxobutoxy)cyclohexyl)oxy)ethyl)-1-ethyl-5- | | |
| | oxo-2-(pyridin-3-yl)pyrrolidine-3-carboxamide. | | |
| 6 | 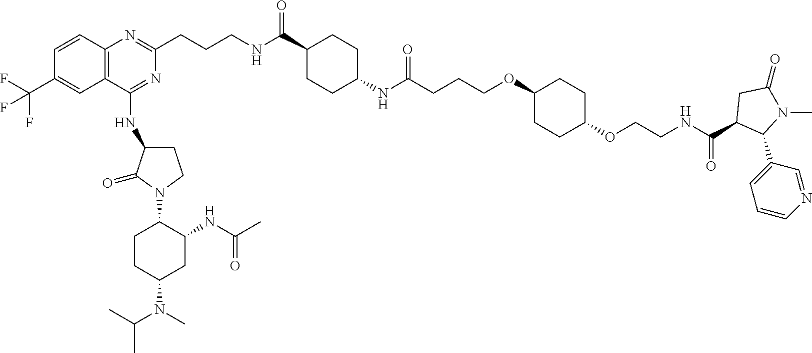 [SMILES: CC(=O)N[C@@H]1C[C@H](N(C)C(C)C)CC[C@@H]1N1CC[C@H](Nc2nc(CCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] | 1119.0 (M + H)+ | ¹H NMR (400 MHz, METHANOL-d₄) δ ppm 8.76-8.73 (m, 1H), 8.60-8.57 (m, 1H), 8.53-8.50 (m, 1H), 8.02-7.98 (m, 1H), 7.86-7.78 (m, 2H), 7.57-7.51 (m, 1H), 5.42-5.34 (m, 1H), 4.86-4.80 (m, 1H), 4.63-4.57 (m, 1H), 4.08-4.00 (m, 1H), 3.67-3.60 (m, 2H), 3.59-3.53 (m, 1H), 3.48 (d, J = 6.4 Hz, 5H), 3.28 (br. s., 3H), 3.10-3.02 (m, 1H), 2.89- 2.80 (m, 3H), 2.77-2.70 (m, 2H), 2.66 (s, 3H), 2.61-2.51 (m, 1H), 2.28-2.19 (m, 7H), 2.15-2.08 (m, 2H), 2.02 (s, 4H), 1.98-1.90 (m, 6H), 1.87-1.79 (m, 3H), 1.77-1.65 (m, 4H), 1.60-1.50 (m, 2H), 1.36-1.20 (m, 8H), 1.12 (d, J = 6.6 Hz, 3H), 1.06 (d, J = 6.4 Hz, 3H), 0.94-0.85 (m, 3H) |
| | (2S,3S)-N-(2-(((1R,4S)-4-(4-(((1S,4R)-4-((3-(4-(((S)-1-((1S,2R,4R)-2-Acetamido-4-(isopropyl(methyl)amino)cyclohexyl)-2- | | |
| | oxopyrrolidin-3-yl)amino)-6-(trifluoromethyl)quinazolin-2-yl)propyl)carbamoyl)cyclohexyl)amino)-4-oxobutoxy)cyclo- | | |
| | hexyl)oxy)ethyl)-1-methyl-5-oxo-2-(pyridin-3-yl)pyrrolidine-3-carboxamide. | | |
| 7 | 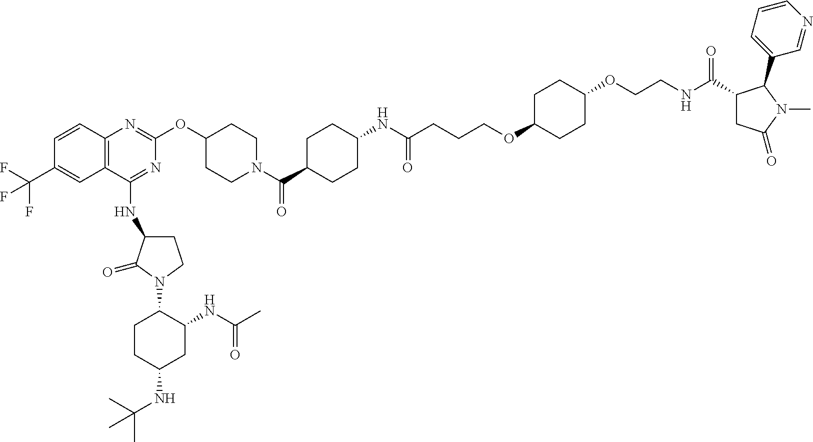 [SMILES: CC(=O)N[C@@H]1C[C@H](NC(C)(C)C)CC[C@@H]1N1CC[C@H](Nc2nc(OC3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] | 1160.8 (M + H)+ | ¹H NMR (METHANOL-d₄) δ ppm 8.58 (m, 1 H) 8.52 (d, 1 H) 8.21 (s, 1 H) 7.78-7.87 (m, 2 H) 7.61 (br s, 1 H) 7.54 (m, 1 H) 5.58-5.69 (m, 1 H) 4.93-5.01 (m, 1 H) 4.83 (d, 1 H) 4.40-4.49 (m, 1H) 3.91-4.09 (m, 3 H) 3.57-3.71 (m, 4 H) 3.46-3.51 (m, 4 H) 3.19-3.30 (m, 4 H) 3.06 (m, 1 H) 2.81 (d, 1 H) 2.64- 2.74 (m, 5 H) 2.54-2.61 (m, 1 H) 2.22- 2.29 (m, 3 H) 1.91-2.02 (m, 11 H) 1.80- 1.88 (m, 7 H) 1.63-1.67 (m, 1 H) 1.15- 1.37 (m, 19 H) 0.85-1.02 (m, 4 H). |
| | (2S,3S)-N-(2-(((1R,4S)-4-(4-(((1S,4R)-4-(4-((4-(((S)-1-((1S,2R,4R)-2-acetamido-4-(tert-butylamino)cyclohexyl)-2-oxopyrrolidin-3- | | |
| | yl)amino)-6-(trifluoromethyl)quinazolin-2-yl)oxy)piperidine-1-carbonyl)cyclohexyl)amino)-4-oxobutoxy)cyclohexyl)oxy)ethyl)- | | |
| | 1-methyl-5-oxo-2-(pyridin-3-yl)pyrrolidine-3-carboxamide | | |
| 8 | 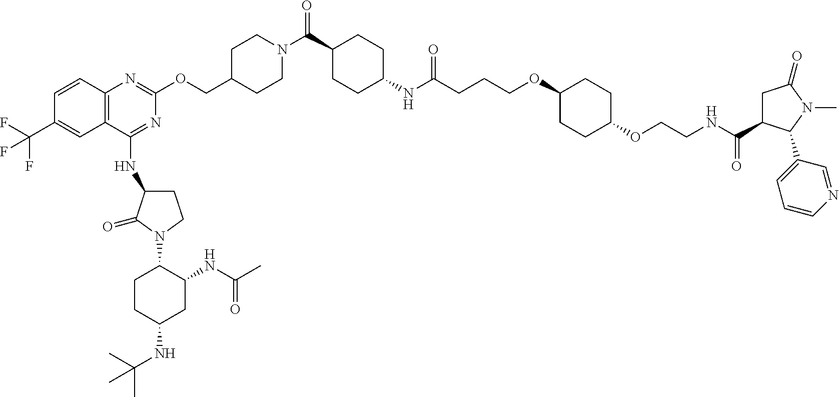 [SMILES: CC(=O)N[C@@H]1C[C@H](NC(C)(C)C)CC[C@@H]1N1CC[C@H](Nc2nc(OCC3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] | 1174.7 (M + H)+ | ¹H NMR (METHANOL-d₄) δ ppm 8.58 (m, 1 H) 8.52 (d, 1 H) 8.19 (s, 1 H) 7.78-7.87 (m, 2 H) 7.57.62 (m, H) 7.55 (d, 1 H) 4.93-5.01 (m, 1 H) 4.83 (d, 1 H) 4.58-4.67 (m, 1 H) 4.40-4.51 (m, 3 H) 4.11-4.17 (m, 1 H) 4.02-4.09 (m, 1 H) 3.58-3.71 (m, 3 H) 3.47 (m, 4 H) 3.23-3.30 (m, 4 H) 3.01-3.11 (m, 1 H) 2.82 (s, 1 H) 2.64-2.74 (m, 6 H) 2.53- 2.61 (m, 1 H) 2.25 (m, 3 H) 1.90-1.99 (m, 10 H) 1.79-1.87 (m, 8 H) 1.57-1.68 (m, 3 H) 1.26-1.34 (m, 9 H) 1.21 (br s, 8 H) 1.00 (d, 1 H) 0.89-0.94 (m, 4 H). |
| | (2S,3S)-N-(2-(((1R,4S)-4-(4-(((1S,4R)-4-(4-(((4-(((S)-1-((1S,2R,4R)-2-Acetamido-4-(tert-butylamino)cyclohexyl)-2-oxopyrrolidin-3- | | |
| | yl)amino)-6-(trifluoromethyl)quinazolin-2-yl)oxy)methyl)piperidine-1-carbonyl)cyclohexyl)amino)-4-oxobutoxy)cyclohexyl)oxy)- | | |
| | ethyl)-1-methyl-5-oxo-2-(pyridin-3-yl)pyrrolidine-3-carboxamide. | | |
| 9 | 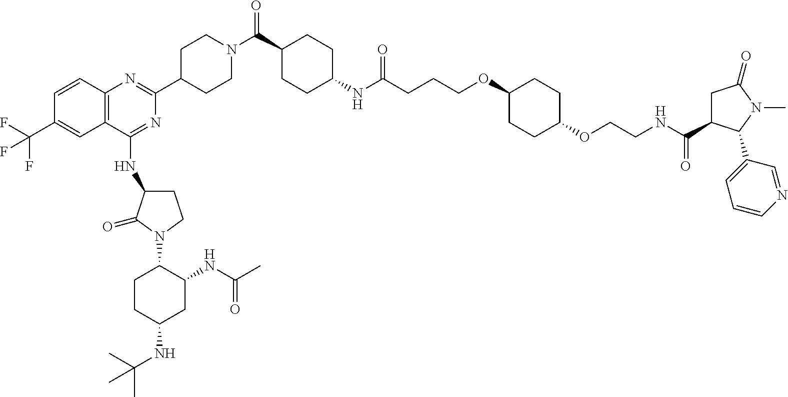 [SMILES: CC(=O)N[C@@H]1C[C@H](NC(C)(C)C)CC[C@@H]1N1CC[C@H](Nc2nc(C3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] | 1144.6 (M + H)+ | ¹H NMR (400 MHz, METHANOL-d₄) δ ppm 1.20 (m, 15 H) 2.03 (m, 25 H) 2.21-2.40 (m, 3 H) 2.45-2.60 (m, 1 H) 2.66 (m, 7 H) 2.98-3.13 (m, 2 H) 3.18- 3.31 (m, 4 H) 3.36-3.77 (m, 9 H) 3.90- 4.07 (m, 1 H) 4.13-4.25 (m, 1 H) 4.49- 4.72 (m, 2 H) 4.80-4.86 (m, 1 H) 5.22- 5.41 (m, 1 H) 7.49-7.58 (m, 1 H) 7.72- 7.83 (m, 1 H) 7.83-7.92 (m, 1 H) 7.95- 8.08 (m, 1 H) 8.45-8.54 (m, 1 H) 8.56- 8.63 (m, 1 H) 8.70-8.81 (m, 1 H) |
| | (2S,3S)-N-(2-(((1R,4S)-4-(4-(((1S,4R)-4-(4-(4-(((S)-1-((1S,2R,4R)-2-Acetamido-4-(tert-butylamino)cyclohexyl)-2-oxopyrrolidin- | | |
| | 3-yl)amino)-6-(trifluoromethyl)quinazolin-2-yl)piperidine-1-carbonyl)cyclohexyl)amino)-4-oxobutoxy)cyclohexyl)oxy)ethyl)- | | |
| | 1-methyl-5-oxo-2-(pyridin-3-yl)pyrrolidine-3-carboxamide. | | |
| 10 | 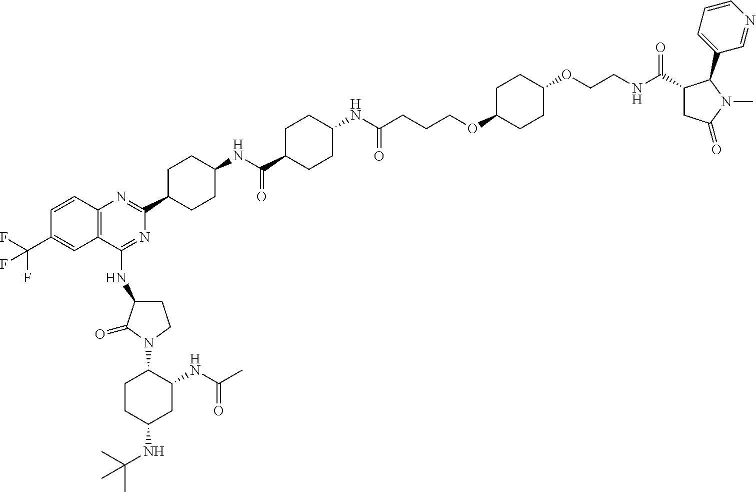 [SMILES: CC(=O)N[C@@H]1C[C@H](NC(C)(C)C)CC[C@@H]1N1CC[C@H](Nc2nc([C@H]3CC[C@@H](NC(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] | 1158.9 (M + H)+ | ¹H NMR (400 MHz, METHANOL-d₄) δ ppm 1.09-1.38 (m, 15 H) 1.53-2.07 (m, 27 H) 2.12-2.39 (m, 6 H) 2.49-2.61 (m, 1 H) 2.66 (m, 4 H) 2.78-2.97 (m, 2 H) 3.00-3.12 (m, 1 H) 3.19-3.33 (m, 4 H) 3.47 (t, J = 5.75 Hz, 8 H) 3.91-4.07 (m, 2 H) 4.54-4.65 (m, 1 H) 4.83 (d, J = 6.60 Hz, 1 H) 5.39 (m, 1 H) 7.49-7.57 (m, 1 H) 7.76-7.83 (m, 1 H) 7.88 (m, 1 H) 7.95-8.04 (m, 1 H) 8.52 (d, J = 1.96 Hz, 1 H) 8.55-8.64 (m, 1 H) 8.76 (s, 1 H) |
| | (2S,3S)-N-(2-(((1R,4S)-4-(4-(((1R,4R)-4-(((1R,4S)-4-(4-(((S)-1-((1S,2R,4R)-2-Acetamido-4-(tert-butylamino) cyclohexyl)- | | |
| | 2-oxopyrrolidin-3-yl)amino)-6-(trifluoromethyl)quinazolin-2-yl)cyclohexyl)carbamoyl)cyclohexyl)amino)-4-oxobutoxy)- | | |
| | cyclohexyl)oxy)ethyl)-1-methyl-5-oxo-2-(pyridin-3-yl)pyrrolidine-3-carboxamide. | | |
| 11 | 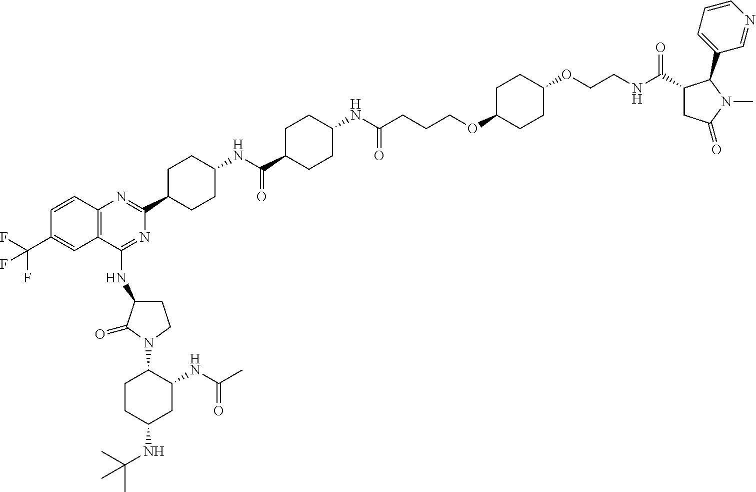 [SMILES: CC(=O)N[C@@H]1C[C@H](NC(C)(C)C)CC[C@@H]1N1CC[C@H](Nc2nc([C@H]3CC[C@H](NC(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] | 1158.8 (M + H)+ | ¹H NMR (400 MHz, METHANOL-d₄) δ ppm 1.19 (s, 9 H) 1.22-1.35 (m, 6 H) 1.36-1.50 (m, 2 H) 1.52-1.68 (m, 2 H) 1.72-2.08 (m, 25 H) 2.11-2.38 (m, 4 H) 2.50-2.62 (m, 1 H) 2.67 (m, 5 H) 2.79- 2.91 (m, 1 H) 3.01-3.12 (m, 1 H) 3.28 (d, J = 4.89 Hz, 4 H) 3.48 (t, J = 5.99 Hz, 9 H) 3.95-4.06 (m, 1 H) 4.53-4.64 (m, 1 H) 4.84 (d, J = 6.85 Hz, 1 H) 5.35 (m, 1 H) 7.50-7.60 (m, 1 H) 7.86 (m, 2 H) 7.93-8.07 (m, 1 H) 8.52 (d, J = 1.96 Hz, 1 H) 8.57-8.64 (m, 1 H) 8.74 (s, 1 H) |
| | (2S,3S)-N-(2-(((1R,4S)-4-(4-(((1S,4R)-4-(((1S,4R)-4-(4-(((S)-1-((1S,2R,4R)-2-Acetamido-4-(tert-butylamino)cyclo- | | |
| | hexyl)-2-oxopyrrolidin-3-yl)amino)-6-(trifluoromethyl)quinazolin-2-yl)cyclohexyl)carbamoyl)cyclohexyl)amino)- | | |
| | 4-oxobutoxy)cyclohexyl)oxy)ethyl)-1-methyl-5-oxo-2-(pyridin-3-yl)pyrrolidine-3-carboxamide. | | |
| 12 | 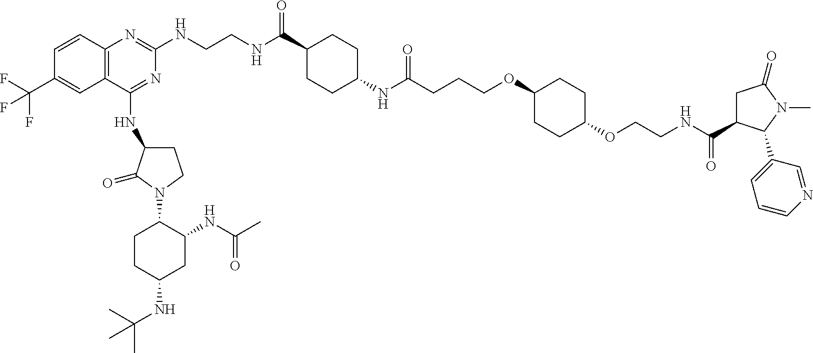 [SMILES: CC(=O)N[C@@H]1C[C@H](NC(C)(C)C)CC[C@@H]1N1CC[C@H](Nc2nc(NCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] | 1118.6 (M + H)+ | ¹H NMR (400 MHz, METHANOL-d₄) δ ppm 8.61-8.56 (m, 1H), 8.52 (d, J = 1.5 Hz, 1H), 8.47 (s, 1H), 7.81 (td, J = 1.9, 7.9 Hz, 1H), 7.76 (dd, J = 2.0, 9.3 Hz, 1H), 7.54 (dd, J = 5.4, 7.8 Hz, 1H), 7.51-7.42 (m, 1H), 5.51 (s, 1H), 4.84 (d, J = 6.8 Hz, 1H), 4.56 (br d, J = 2.0 Hz, 1H), 4.07-3.99 (m, 1H), 3.67-3.53 (m, 4H), 3.52-3.36 (m, 6H), 3.31-3.21 (m, 4H), 3.10-3.01 (m, 1H), 2.88-2.79 (m, 1H), 2.75-2.68 (m, 1H), 2.66 (s, 3H), 2.23 (t, J = 7.3 Hz, 2H), 2.03 (s, 2H), 1.99-1.79 (m, 10H), 1.78-1.68 (m, 2H), 1.60-1.49 (m, 2H), 1.35-1.22 (m, 8H), 1.19 (s, 9H) |
| | (2S,3S)-N-(2-(((1R,4S)-4-(4-(((1S,4R)-4-((2-((4-(((S)-1-((1S,2R,4R)-2-Acetamido-4-(tert-butylamino)cyclohexyl)-2-oxopyrrolidin- | | |
| | 3-yl)amino)-6-(trifluoromethyl)quinazolin-2-yl)amino)ethyl)carbamoyl)cyclohexyl)amino)-4-oxobutoxy)cyclohexyl)oxy)- | | |
| | ethyl)-1-methyl-5-oxo-2-(pyridin-3-yl)pyrrolidine-3-carboxamide. | | |
| 13 | 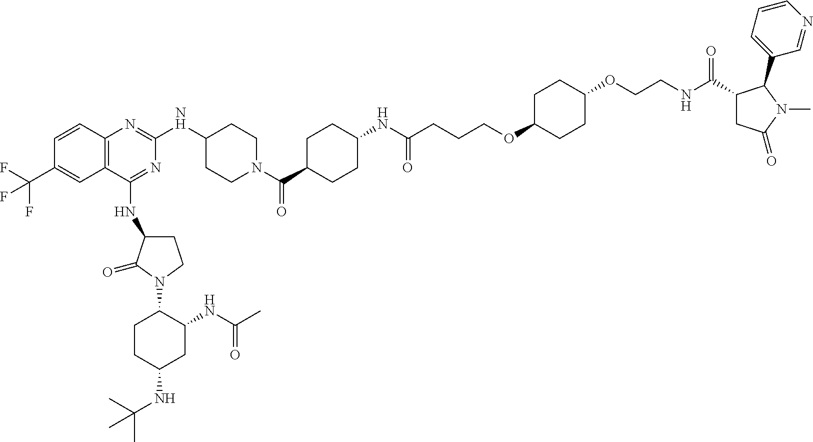 [SMILES: CC(=O)N[C@@H]1C[C@H](NC(C)(C)C)CC[C@@H]1N1CC[C@H](Nc2nc(NC3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] | 1159.7 (M + H)+ | ¹H NMR (400 MHz, DICHLORO- METHANE-d₂) δ ppm 8.82-8.99 (m, 1 H) 8.63 (dd, J = 4.6, 1.7 Hz, 1 H) 8.55 (d, J = 2.0 Hz, 1 H) 7.78 (br s, 1 H) 7.61 (dt, J = 8.2, 2.0 Hz, 1 H) 7.49 (br d, J = 8.8 Hz, 1 H) 7.39 (dd, J = 8.1, 4.6 Hz, 1 H) 5.75 (br s, 1 H) 5.46-5.66 (m, 1 H) 5.37-5.39 (m, 1 H) 4.96 (br s, 1 H) 4.68-4.86 (m, 2 H) 4.30-4.49 (m, 1 H) 4.25 (br dd, J = 6.6, 2.2 Hz, 1 H) 4.02 (br d, J = 12.7 Hz, 1 H) 3.92 (br d, J = 12.2 Hz, 1 H) 3.62-3.83 (m, 1 H) 3.29- 3.57 (m, 7 H) 3.12-3.29 (m, 3 H) 2.99 (br s, 1 H) 2.63-2.85 (m, 6 H) 2.40- 2.58 (m, 1 H) 2.32 (br d, J = 7.3 Hz, 1 H) 2.22 (t, J = 7.1 Hz, 2 H) 1.99-2.13 (m, 7 H) 1.78-1.99 (m, 11 H) 1.55-1.76 (m, 6 H) 1.14-1.35 (m, 14 H) 0.89 (br d, J = 18.6 Hz, 2 H). |
| | (2S,3S)-N-(2-(((1R,4S)-4-(4-(((1S,4R)-4-(4-((4-(((S)-1-((1S,2R,4R)-2Acetamido-4-(tert-butylamino)cyclohexyl)-2- | | |
| | oxopyrrolidin-3-yl)amino)-6-(trifluoromethyl)quinazolin-2-yl)amino)piperidine-1-carbonyl)cyclohexyl)amino)- | | |
| | 4-oxobutoxy)cyclohexyl)oxy)ethyl)-1-methyl-5-oxo-2-(pyridin-3-yl)pyrrolidine-3-carboxamide. | | |
| 14 | 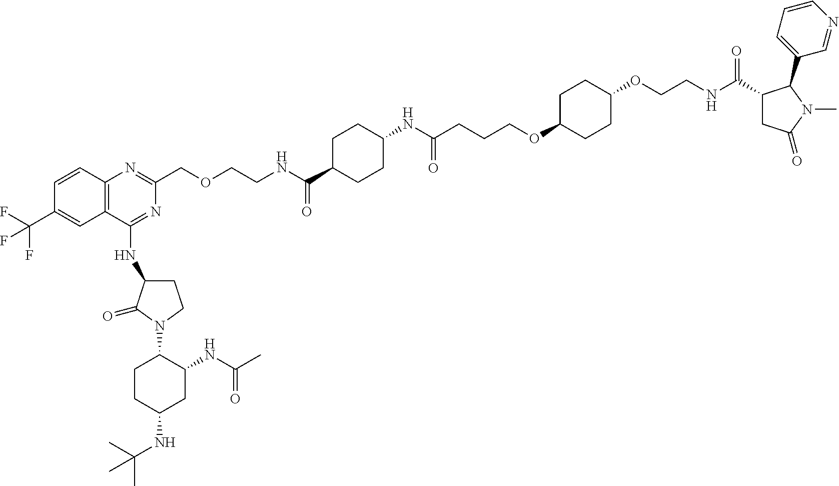 [SMILES: CC(=O)N[C@@H]1C[C@H](NC(C)(C)C)CC[C@@H]1N1CC[C@H](Nc2nc(COCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] | 1135.0 (M + H)+ | ¹H NMR (400 MHz, METHANOL-d₄) δ ppm 0.86-0.95 (m, 5 H) 0.96-1.02 (m, 1 H) 1.19 (s, 9 H) 1.29 (br s, 9 H) 1.24- 1.36 (m, 1 H) 1.53-1.61 (m, 2 H) 1.71- 1.77 (m, 2 H) 1.78-1.99 (m, 14 H) 2.01- 2.06 (m, 3 H) 2.18-2.27 (m, 3 H) 2.50- 2.59 (m, 1 H) 2.64-2.74 (m, 4 H) 2.79- 2.87 (m, 1 H) 3.06 (ddd, J = 9.54, 8.07, 6.85 Hz, 1 H) 3.15-3.30 (m, 1 H) 3.20- 3.29 (m, 2 H) 3.35-3.40 (m, 1 H) 3.41- 3.50 (m, 1 H) 3.45-3.50 (m, 4 H) 3.54- 3.67 (m, 3 H) 3.71-3.76 (m, 2 H) 3.96- 4.04 (m, 1 H) 4.55-4.60 (m, 1 H) 4.64 (s, 2 H) 4.81-4.86 (m, 1 H) 5.33-5.41 (m, 1 H) 5.51 (s, 1 H) 7.47-7.58 (m, 1 H) 7.76-7.85 (m, 1 H) 7.91-7.98 (m, 1 H) 8.01-8.07 (m, 1 H) 8.49-8.54 (m, 1 H) 8.57-8.61(m, 1 H) 8.74-8.85 (m, 1 H) |
| | (2S,3S)-N-(2-(((1R,4S)-4-(4-(((1S,4R)-4-((2-((4-(((S)-1-((1S,2R,4R)-2-acetamido-4-(tert-butylamino)cyclohexyl)-2-oxopyrrolidin-3- | | |
| | yl)amino)-6-(trifluoromethyl)quinazolin-2-yl)methoxy)ethyl)carbamoyl)cyclohexyl)amino)-4-oxobutoxy)cyclohexyl)oxy)ethyl)- | | |
| | 1-methyl-5-oxo-2-(pyridin-3-yl)pyrrolidine-3-carboxamide. | | |

### Biological Assays

Example Compounds 1-14 which are compounds of Formula (I) having a CCR2 binding moiety were tested in various biological assays as described in more detail below.

### Example 15—Antibody Dependent Cellular Cytotoxicity (ADCC) Reporter Assay

An antibody dependent cellular cytoxocity reporter assay was conducted using the following four assay components: (i) ARM compound of Formula (I) targeting CCR2 (concentrations ranging from 1 pM to 10 μM) (ii) anti-cotinine antibody having a heavy chain sequence of SEQ ID NO: 11 and a light chain sequence of SEQ ID NO: 12 (rabbit variable region with human IgG1 Fc domain containing a DE mutation (S239D/I332E)) (concentrations ranging from 0.01 μg/mL to 200 μg/mL); (iii) target cells: CHOK1 cells engineered to overexpress either human CCR2 (typically 1000-20,000 cells perwell) and (iv) reporter cells: Jurkat cells engineered to express FcγRIIIa with the reporter gene luciferase under the control of the NFAT promoter (typically 3000-75,000 cells per well). Reagents were combined in a final volume of 20 μL in a 384—well tissue culture treated plate. All four assay components were incubated together for about 12-18 hours. Thereafter, BioGlo Detection reagent (Promega) was added to the wells to lyse the cells and provide a substrate for the luciferase reporter protein. Luminescence signal was measured on a microplate reader and signal:background was calculated by dividing the signal of a test well by the signal obtained when no ARM compound of Formula (I) was added. EC50 calculations were done using Graphpad Prism Software, specifically a nonlinear regression curve fit (Y=Bottom+(Top−Bottom)/(1+10 \hat{ }((Log EC50−X)*HillSlope))).

ARMs compounds of Formula (I) of the invention were tested for ADCC activity in the above assay in or more experimental runs and the results are shown in Table 3 below. Potency of the compounds of Formula (I) of the invention is reported as a pEC50 value. The pEC50 value is the negative log of the EC50 value, wherein the EC50 value is the half maximal effective concentration measured in molar (M). For compounds tested in more than one experimental run, the pEC50 value is reported as an average.

TABLE 3
Results for ADC Reporter Assay (Example 15)

| Compound | |
|---|---|
| Example No. | ADCC Reporter Assay pEC50 |
| 1 | 9.8 |
| 2 | 9.5 |
| 3 | 9.8 |
| 4 | 9.6 |
| 5 | 8.5 |
| 6 | 9.8 |
| 7 | 7.1 |
| 8 | 7.3 |
| 9 | 9.2 |
| 10 | 9.6 |
| 11 | 9.8 |
| 12 | 9.6 |
| 13 | 9.5 |
| 14 | 9.3 |

### Example 16—In Vivo Depletion Assay

Human CCR2 (hCCR2) knock-in mice (C57 background) were dosed intravenously with a PBS solution containing a compound of Formula (I) to measure depletion of CCR2 expressing cells in peripheral blood. Peripheral blood from IV dosed mice was also analyzed to determine PK properties of the compounds of Formula (I).

Stock solution preparation: Stock solutions of compounds of Formula (I) were prepared at 20 mg/mL and 100 mg/mL in DMSO for PK/PD and dose tolerability studies respectively. Stock solutions were stored at −20° C. until further usage.

Formulations preparation: On the day of experiment, the stock solution of the compound of Formula (I) was removed from storage at −20° C. and thawed at room temperature. Anti-cotinine antibody having a heavy chain sequence of SEQ ID NO: 13 and a light chain sequence of SEQ ID NO: 14 (rabbit variable region sequence with mouse IgG2a Fc domain), if required was removed from storage at −80° C. and thawed at room temperature. Antibody vials were immediately transferred into wet ice after thawing. Compounds of Formula (I) were further diluted in DMSO as per experimental requirements.

Formulation composition: The formulation composition was Saline: DMSO: PBS. Saline was added based on the quantity required and then stock solution of the compound of Formula (1) prepared in DMSO, followed by addition of antibody in PBS. Formulations were incubated at room temperature for 30 minutes before administration to the mouse. DMSO was used at 1 to 2% (v/v) in the final formulation.

Administration to Animal: Solution formulation of antibody and compound of Formula (I) was injected (bolus injection) to the restrained mouse in the right/left lateral tail vein. Animals were dosed with 0.1 milligram per kilogram (mpk) of the compound of Formula (I) and 10 mpk of the anti-cotinine antibody.

Collection of Blood for PK: Blood was collected at time points 0.25 hour, 2 hours, and 4 hours following administration (50 μL/time point) through retro-orbital bleeding under mild isoflurane anesthesia.

Terminal bleeding at end of experiment (48 hr): Approximately 250 μL of blood in K₂EDTA tube and approximately 250 μL of blood in SST (serum separation tube) was collected from each mouse through retro-orbital bleeding under deep isoflurane anesthesia. After bleeding, each mouse was sacrificed by cervical dislocation. The blood distribution at termination was determined as follows:
For PK: 50 μL of K₂EDTA blood was transferred to another tube for PK
Flow cytometry: Remaining blood (˜200 μL) was used for flow cytometry analysis.
For serum collection: serum separator tubes (BD) were centrifuged at 4° C., 5000 rpm for 15 minutes. Approximately 100 μL serum was separated and stored at −80° C. for further usage.
Blood drug concentration: Samples collected at the various timepoints (0.25 hr, 2 hr, 24 and 48 hr) were analyzed to determine blood drug concentration.

Flow cytometry analysis: CCR2 depletion in peripheral blood was determined by flow cytometry. Briefly, peripheral blood was collected from all animals at the end of study termination and processed. Blood samples were lysed using 1×RBC Lysis buffer and the resulting cell pellet was washed twice in FACS buffer (HBSS containing 5% FBS). Cells were subjected to Fc blocking using mouse Fc block from Biolegend (Trustain, anti-mouse CD16/32, Cat: 101319) to block non-specific binding followed by surface staining for immune markers (CD45, CD3, CD11b, Ly6C, human CCR2). Surface staining was done for 30 minutes in ice and cells were washed twice to remove unbound antibodies. Cells were stained with 7AAD to determine viability and acquired on BD FACSVerse flow cytometer. Appropriate FMO controls and single-color controls were included in the experiment for gating and compensation respectively.

CCR2+monocytes were gated as CD11b+Ly6C+Hi, medium and Low populations and treatment induced reduction in CD11b+Ly6c+Hi cells (i.e., cells with high CCR2 expression) was evaluated. Percent depletion of CD11b+Ly6c+Hi cells and CCR2 expression for the different treatment groups was determined. The results are shown below in Table 4 and are reported as percent (%) depletion of CCR2 expressing cells.

Blood drug concentration analysis: Drug concentration in blood samples was determined by an LC-MS/MS-based bioanalytical method developed at Syngene. Samples were analyzed on Q-Trap, API-5500 LC-MS/MS system coupled with Exion UHPLC system from SCIEX, USA operated in multiple reaction monitoring mode employing electrospray ionization technique in positive polarity. Analyte and internal standard peaks were resolved on Synergi Polar, 75×2.0 mm, 4 μcolumn using mobile phase 10 mM Ammonium acetate in Milli-Q water as phase A and 0.1% Formic acid in acetonitrile as Phase B. Gradient elution was performed with initial composition 95% Phase A at 0.0 min, holding it for 0.2 minutes, ramping to 5% by 1.0 minute, keeping the same for next 0.5 minutes and coming back to 95% by 1.6 minutes. The total run time was 2 minutes.

Working dilutions for calibration curve and quality control standards were prepared by serially diluting 20 mg/mL stock solution with DMSO. Spiked concentrations for calibration curve in the whole blood ranged from 1 ng/mL to 1000 ng/mL. The working solution of internal standard (Verapamil, 25 ng/mL) was prepared in acetonitrile. 10 μL of the study sample and calibration curve, quality control, and blank whole blood samples were aliquoted in 96 deep well plates for processing. 10 μL of Milli-Q water was added to all the samples and briefly vortexed to initiate complete hemolysis. 10 μL of 20 mM dithiothreitol (DTT) was added to all the samples and incubated for 30 minutes at 37° C. The addition of OTT enhanced the recovery of ARM compounds of Formula (I) from the biological matrix. 300 μL of working internal standard solution was added to all samples except total blank and wash samples, where 300 μL of acetonitrile was added. All the samples were vortex mixed for 5 minutes, followed by centrifugation at 4000 rpm for 10 minutes at 4° C. Supernatants were transferred to the loading plate and injected 3 μL to LC-MS/MS system for analysis. The results are shown below in Table 4 and are reported as compound exposure 48 hours post-dose.

TABLE 4
Results for In Vivo Depletion Assay- % Depletion
of CCR2 expressing cells and PK analysis of ARM
compounds of Formula (I) after 48 hours exposure

| | | Exposure of Compound of |
|---|---|---|
| Compound | % Depletion of CCR2 | Formula (I) 48 hours |
| Example No. | expressing cells | post-dose (ng/mL) |
| 1 | 89.18 | 42.91 |
| 3 | 84 | 37.08 |
| 10 | 89.71 | 35.47 |
| 11 | 95.41 | 93.43 |

### Example 17—Nicotinic Acetylcholine Receptor Antagonism Assay

Cells expressing human alpha1 nicotinic acetylcholine (nACh) receptor (nAChR) were washed and dissociated using Trypsin containing buffer. Cells were then washed and seeded at a density of 3-5 million cells/mL and allowed to grow for 3 days. Then, 60-80 μL of cells were added to plates and incubated with dose response of an ARM compound of Formula (I) with a top concentration of either 10 μM or 100 μM. The receptor was then stimulated via addition of acetylcholine (ACh) and activation of the receptor was monitored via the IONFlux automated patch clamp system. IonFlux Data Analyzer software was utilized to perform leak subtraction and measure (min) peak current amplitude. These data were then exported to Excel.

Data processing: (1) Current values recorded for vehicle only applications in individual traps/patterns were subtracted from corresponding current amplitude values for control, test article and control+test article. (2) Control response was calculated as % of the average response of the 3rd out of total 3 ACh (10 mM) applications alone. In rare situations, if the 3rd ACh response was obscured due to technical artifacts, the 2nd and/or 1st ACh (10 mM) application was considered as control. The peak current amplitude generated in the presence of each test article concentration was then used to calculate the % inhibition as compared to control response (10 mM ACh alone). These estimates were further corrected for vehicle response and run down corrected by normalizing with time matched control responses (10 mM ACh alone). The corrected percentages were then fit with a non-linear function in GraphPad Prism 6 (or later versions) to determine the IC50.

Exclusion criteria: (i) any n's with poor recording quality, (ii) control current amplitude <500 pA or (iii) significant outliers, as determined by the Grubbs' test.

ARMs compounds of Formula (I) of the invention were tested for antagonism of the nAChR in the above assay in or more experimental runs and the results are shown in Table 5 below. Antagonistic activity of the compounds of Formula (I) of the invention is reported as an IC50 value (half maximal inhibitory concentration) and the maximum percentage antagonism observed at the highest concentration of compound of Formula (I) tested, as indicated in Table 5. For compounds tested in more than one experimental run, the IC50 value is reported as an average.

TABLE 5
Results of nAChR Antagonism Assay

| Compound | IC₅₀ | Maximum Concentration of | Maximum |
|---|---|---|---|
| Example No. | (μM) | Compound Tested (μM) | Response (%) |
| 2 | >11.11 | 11.11 | 9 |
| 3 | >33.3 | 33.3 | 38 |
| 4 | >100 | 100 | 48 |
| 5 | >33.3 | 33.33 | 16 |
| 6 | >33.3 | 33.3 | 48 |
| 9 | >11.1 | 11.1 | 2 |
| 10 | >33.3 | 33.3 | 28 |
| 11 | >33.3 | 33.3 | 47 |
| 13 | 10.1 | 33.33 | 68 |
| 14 | >100 | 100 | 54 |

### SEQUENCE LISTINGS

Heavy chain CDR1 amino acid sequence
SEQ ID NO: 1
NYWMS
Heavy chain CDR2 amino acid sequence
SEQ ID NO: 2
DIHGNRGFNYHASWAKG
Heavy chain CDR3 amino acid sequence
SEQ ID NO: 3
ADDSGSHDI
Light chain CDR1 amino acid sequence
SEQ ID NO: 4
QSSQSVYSAKLS
Light chain CDR2 amino acid sequence
SEQ ID NO: 5
YGSTLAS
Light chain CDR3 amino acid sequence
SEQ ID NO: 6
QGTFYGPDWYFA
Variable heavy chain amino acid sequence
SEQ ID NO: 7
EVQLVESGGGLVQPGGSLRLSCAASGFTFSNYWMSWVRQAPGKG
LEWVGDIHGNRGFNYHASWAKGRFTVSRSKNTLYLQMNSLRAED
TAVYYCAKADDSGSHDIWGQGTLVTVSS
Variable light chain amino acid sequence
SEQ ID NO: 8
DIQMTQSPSSLSASVGDRVTITCQSSQSVYSAKLSWYQQKPGKA
PKLLIYYGSTLASGVPSRFSGSGSGTQFTLTISSLQPEDFATYY
CQGTFYGPDWYFAFGGGTKVEIK
Heavy chain amino acid sequence
SEQ ID NO: 9
EVQLVESGGGLVQPGGSLRLSCAASGFTFSNYWMSWVRQAPGKG
LEWVGDIHGNRGFNYHASWAKGRFTVSRSKNTLYLQMNSLRAED
TAVYYCAKADDSGSHDIWGQGTLVTVSSASTKGPSVFPLAPSSK
STSGGTAALGCLVKDYFPEPVTVSWNSGALTSGVHTFPAVLQSS
GLYSLSSVVTVPSSSLGTQTYICNVNHKPSNTKVDKKVEPKSCD
KTHTCPPCPAPELLGGPDVFLFPPKPKDTLMISRTPEVTCVVVD
VSHEDPEVKFNWYVDGVEVHNAKTKPREEQYNSTYRVVSVLTVL
HQDWLNGKEYKCKVSNKALPAPEEKTISKAKGQPREPQVYTLPP
SRDELTKNQVSLTCLVKGFYPSDIAVEWESNGQPENNYKTTPPV
LDSDGSFFLYSKLTVDKSRWQQGNVFSCSVMHEALHNHYTQKSL
SLSPGK
Light chain amino acid sequence
SEQ ID NO: 10
DIQMTQSPSSLSASVGDRVTITCQSSQSVYSAKLSWYQQKPGKA
PKLLIYYGSTLASGVPSRFSGSGSGTQFTLTISSLQPEDFATYY
CQGTFYGPDWYFAFGGGTKVEIKRTVAAPSVFIFPPSDEQLKSG
TASVVCLLNNFYPREAKVQWKVDNALQSGNSQESVTEQDSKDST
YSLSSTLTLSKADYEKHKVYACEVTHQGLSSPVTKSFNRGEC
Heavy chain amino acid sequence
SEQ ID NO: 11
QQQLVESGGR LVTPGGSLTL TCTASGFSLN NYWMSWVRQA
PGKGLEWIGD IHGNRGFNYH ASWAKGRFTV SRTSTTVDLR
MTSLTTEDTA IYFCARADDS GSHDIWGPGT LVTVSSASTK
GPSVFPLAPS SKSTSGGTAA LGCLVKDYFP EPVTVSWNSG
ALTSGVHTFP AVLQSSGLYS LSSVVTVPSS SLGTQTYICN
VNHKPSNTKV DKKVEPKSCD KTHTCPPCPA PELLGGPDVF
LFPPKPKDTL MISRTPEVTC VVVDVSHEDP EVKFNWYVDG
VEVHNAKTKP REEQYNSTYR VVSVLTVLHQ DWLNGKEYKC
KVSNKALPAP EEKTISKAKG QPREPQVYTL PPSRDELTKN
QVSLTCLVKG FYPSDIAVEW ESNGQPENNY KTTPPVLDSD
GSFFLYSKLT VDKSRWQQGN VFSCSVMHEA LHNHYTQKSL
SLSPGK
Light chain amino acid sequence
SEQ ID NO: 12
ELDLTQTPSPVSAAVGDTVTINCQSSQSVYSAKLSWYQQKPGQP
PKLLIYYGSTLASGVPSRFKGSGSGTQFSLTISDVQCADAATYY
CQGTYYGPDWYFAFGGGTEVVVKRTVAAPSVFIFPPSDEQLKSG
TASVVCLLNNFYPREAKVQWKVDNALQSGNSQESVTEQDSKDST
YSLSSTLTLSKADYEKHKVYACEVTHQGLSSPVTKSFNRGEC
Heavy chain amino acid sequence
SEQ ID NO: 13
QQQLVESGGRLVTPGGSLTLTCTASGFSLNNYWMSWVRQAPGKG
LEWIGDIHGNRGFNYHASWAKGRFTVSRTSTTVDLRMTSLTTED
TAIYFCARADDSGSHDIWGPGTLVTVSSAKTTAPSVYPLAPVCG
DTTGSSVTLGCLVKGYFPEPVTLTWNSGSLSSGVHTFPAVLQSD
LYTLSSSVTVTSSTWPSQSITCNVAHPASSTKVDKKIEPRGPTI
KPCPPCKCPAPNLLGGPSVFIFPPKIKDVLMISLSPMVTCVVVD
VSEDDPDVQISWFVNNVEVLTAQTQTHREDYNSTLRVVSALPIQ
HQDWMSGKEFKCKVNNKALPAPIERTISKPKGSVRAPQVYVLPP
PEEEMTKKQVTLTCMVTDFMPEDIYVEWTNNGKTELNYKNTEPV
LDSDGSYFMYSKLRVEKKNWVERNSYSCSVVHEGLHNHHTTKSF
SRTPGK
Light chain amino acid sequence
SEQ ID NO: 14
ELDLTQTPSPVSAAVGDTVTINCQSSQSVYSAKLSWYQQKPGQP
PKLLIYYGSTLASGVPSREKGSGSGTQFSLTISDVQCADAATYY
CQGTYYGPDWYFAFGGGTEVVVKRADAAPTVSIFPPSSEQLTSG
GASVVCFLNNFYPKDINVKWKIDGSERQNGVLNSWTDQDSKDST
YSMSSTLTLTKDEYERHNSYTCEATHKTSTSPIVKSFNRNEC

1. A compound of Formula (I): 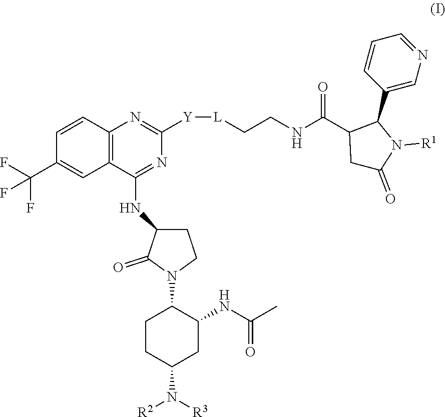
or a pharmaceutically acceptable salt thereof,
wherein:
R¹ is C₁₋₄ alkyl or C₃₋₆ cycloalkyl;
R² is hydrogen or C₁₋₄ alkyl;
R³ is hydrogen or C₁₋₄ alkyl;
L is a divalent linker of Formula (L-a): 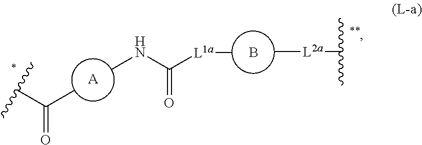
or a stereoisomer thereof,
wherein:
Ring A and Ring B are each independently C₄₋₆ cycloalkylene;
L^{1a} is C₃₋₅ linear alkylene, wherein 1 or 2 methylene units are replaced with —O— or —NR^{a}—;
each R^{a} is independently hydrogen or C₁₋₃ alkyl; and
L^{2a} is —O—, —NHC(O)—, or —CH₂—O—;
wherein 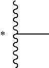
represents a covalent bond to the Y group of Formula (I), or when Y is a bond, a covalent bond to the quinazoline group of Formula (I), and 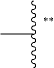
represents a covalent bond to the methylene group of Formula (I); and
Y is a bond or a divalent spacer moiety of one to twelve atoms in length.

2. The compound of claim 1, or a pharmaceutically acceptable salt thereof, wherein R¹ is —CH₃.

3. The compound of claim 1, or a pharmaceutically acceptable salt thereof, wherein R¹ is ethyl.

4. The compound of claim 1, or a pharmaceutically acceptable salt thereof, wherein R² is isopropyl and R³ is methyl.

5. The compound of claim 1, or a pharmaceutically acceptable salt thereof, wherein R² is t-butyl and R³ is hydrogen.

6. The compound of claim 1, or a pharmaceutically acceptable salt thereof, wherein L is a divalent linker of Formula (L-a-i): 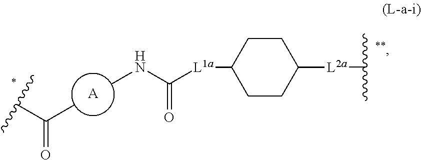
or a stereoisomer thereof,
wherein Ring A, L^{1a}, L^{2a}, 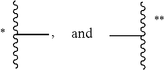
are as defined for Formula (L-a).

7. The compound of claim 1, or a pharmaceutically acceptable salt thereof, wherein L is a divalent linker of Formula (L-a-ii): 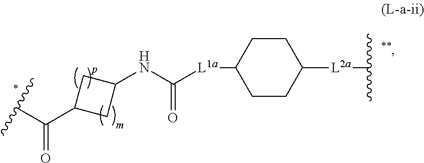
or a stereoisomer thereof,
wherein L^{1a}, L^{2a}, 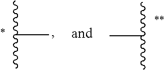
are as defined for Formula (L-a); p is 1 or 2; and m is 1 or 2.

8. The compound of claim 1, or a pharmaceutically acceptable salt thereof, wherein L is a divalent linker of Formula (L-a-iii): 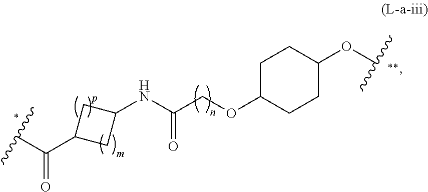
or a stereoisomer thereof,
wherein p is 1 or 2; m is 1 or 2; n is 1, 2, or 3; and 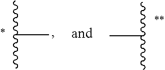
are as defined for Formula (L-a).

9. The compound of claim 1, or a pharmaceutically acceptable salt thereof, wherein L is a divalent linker of Formula (L-a) selected from the group consisting of:

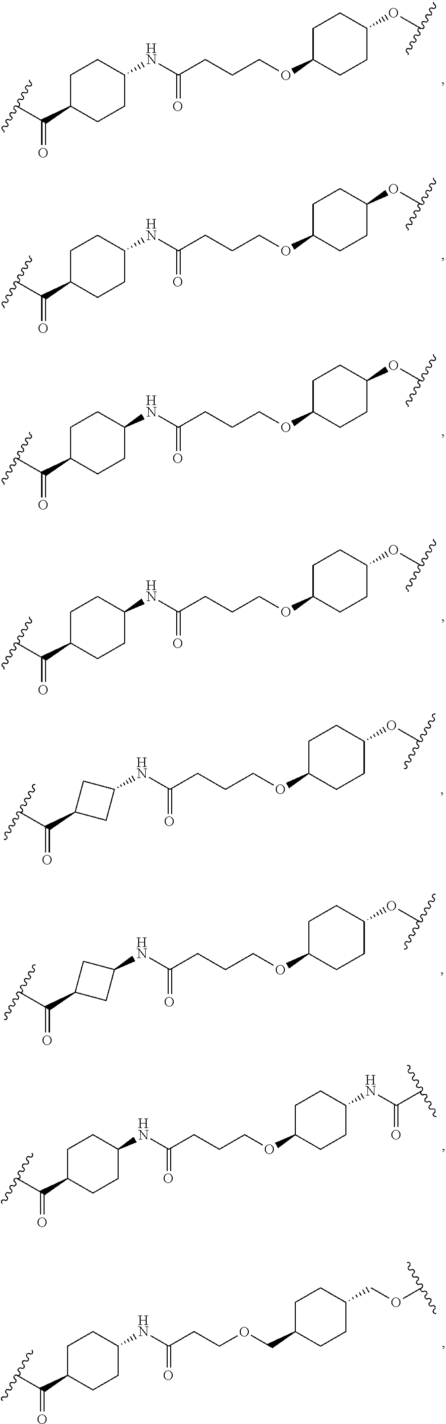

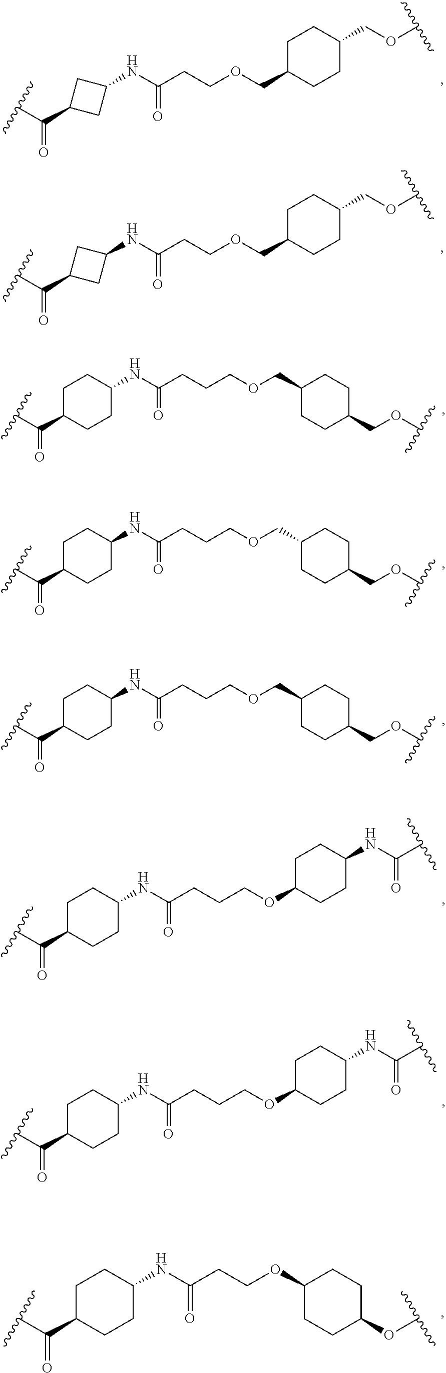

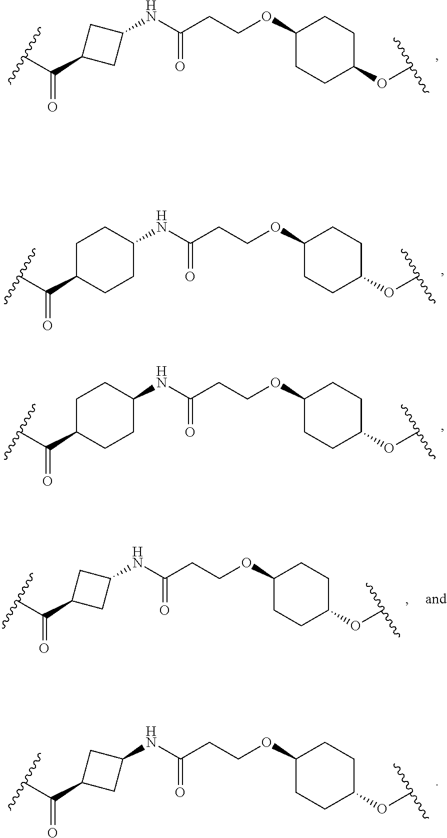 [SMILES: *O[C@H]1CC[C@@H](OCCC(=O)N[C@H]2C[C@H](C(*)=O)C2)CC1 ; *O[C@H]1CC[C@H](OCCC(=O)N[C@H]2CC[C@H](C(*)=O)CC2)CC1 ; *O[C@H]1CC[C@H](OCCC(=O)N[C@H]2CC[C@@H](C(*)=O)CC2)CC1 ; *O[C@H]1CC[C@H](OCCC(=O)N[C@H]2C[C@H](C(*)=O)C2)CC1 ; *O[C@H]1CC[C@H](OCCC(=O)N[C@H]2C[C@@H](C(*)=O)C2)CC1]

10. The compound of claim 1, or a pharmaceutically acceptable salt thereof, wherein Y is selected from a bond; —NH—; —(C₁₋₁₂ alkylene)-, wherein 1, 2, or 3 methylene units are replaced with —O—, —NH—, —C(O)—, —NHC(O)—, —C(O)NH—, —(C₃₋₆ cycloalkylene)-, —(C₃₋₆ cycloalkenylene)-, 3- to 6-membered heterocycloalkylene, arylene, or heteroarylene; or —(C₂₋₁₂ alkenylene)-, wherein 1, 2, or 3 methylene units are replaced with —O—, —NH—, —C(O)—, —NHC(O)—, —C(O)NH—, —(C₃₋₆ cycloalkylene)-, —(C₃₋₆ cycloalkenylene)-, 3-to 6-membered heterocycloalkylene, arylene, or heteroarylene.

11. (canceled)

12. The compound of claim 9, or a pharmaceutically acceptable salt thereof, wherein Y is selected from the group consisting of:

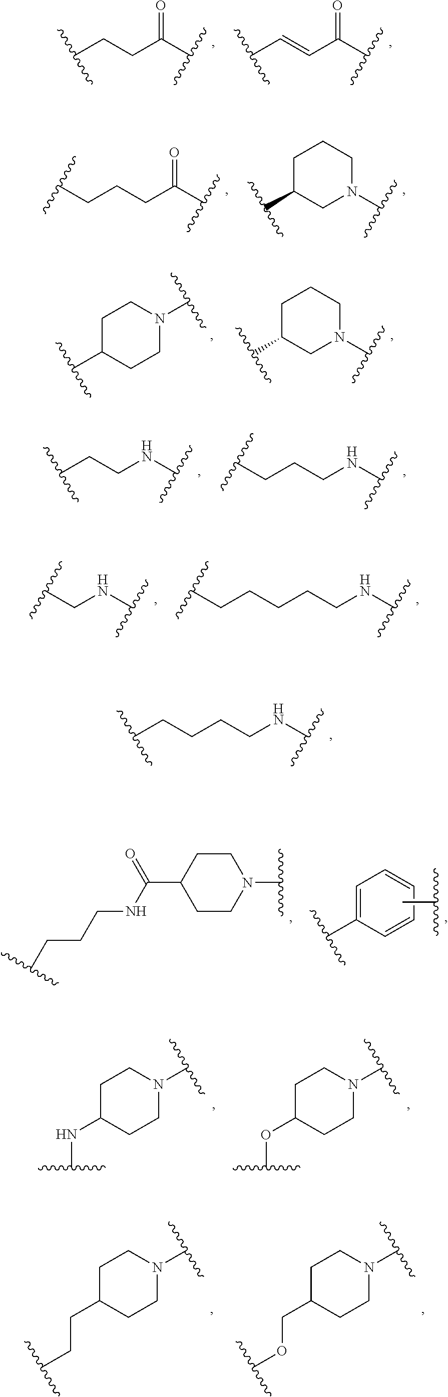

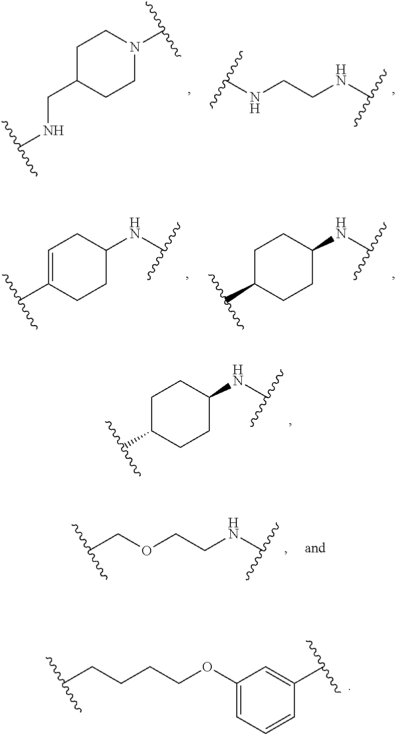

13. The compound of claim 1, wherein the compound is a compound in Table 1 or a pharmaceutically acceptable salt thereof.

14. A compound selected from the group consisting of: 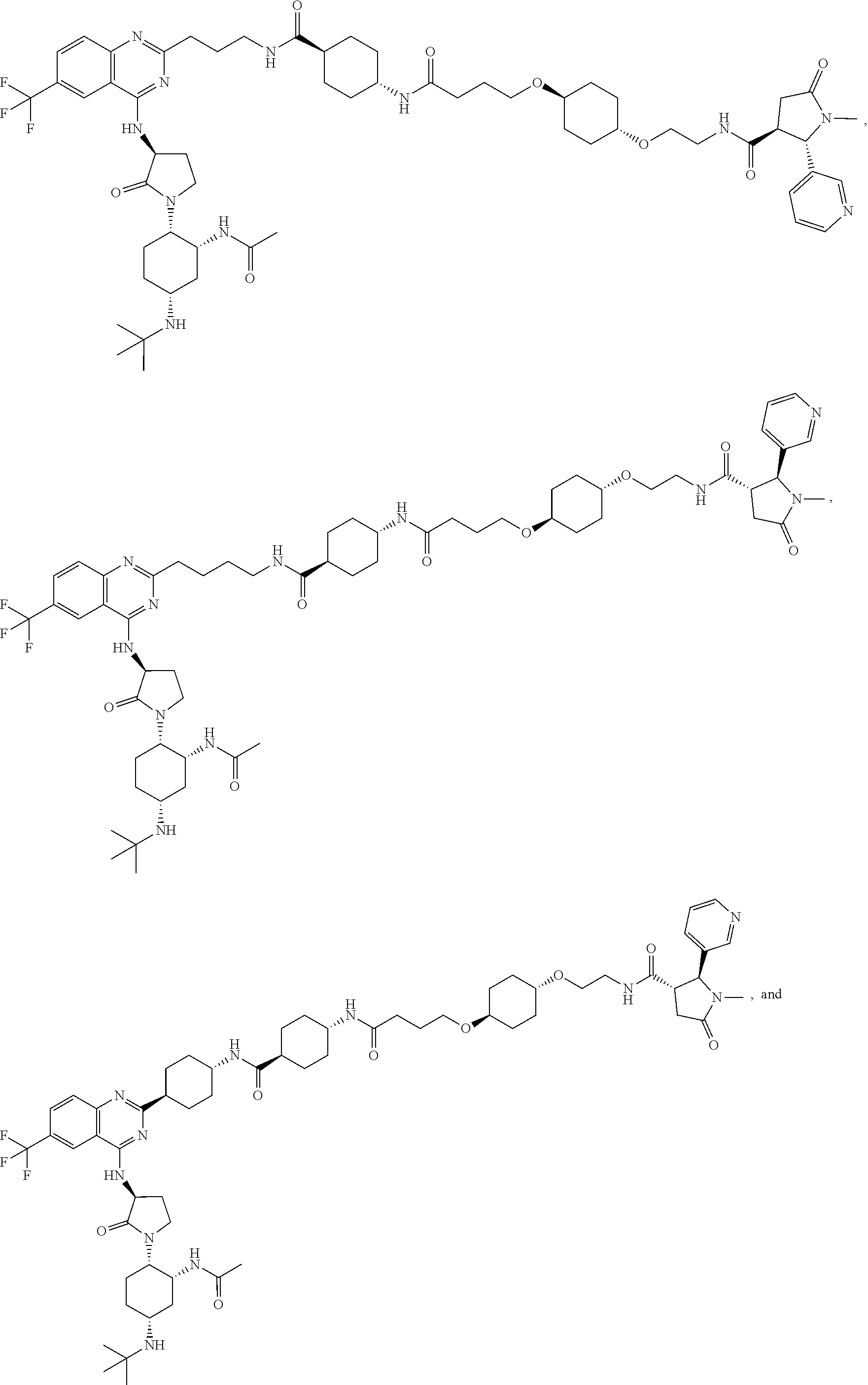 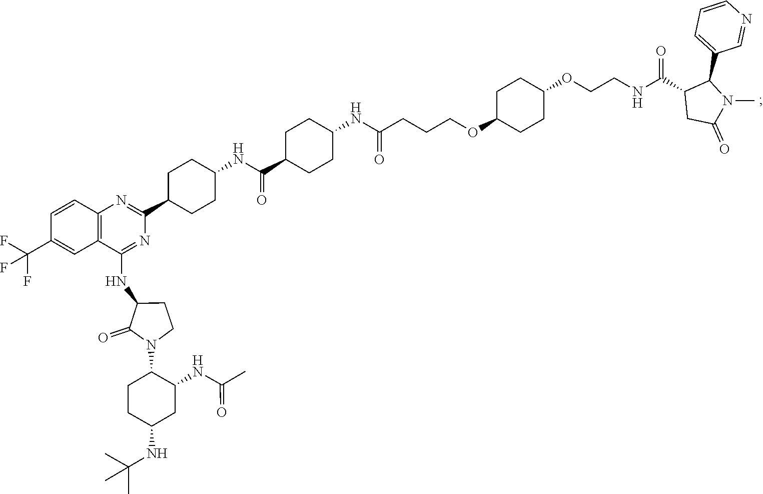 [SMILES: CC(=O)N[C@@H]1C[C@H](NC(C)(C)C)CC[C@@H]1N1CC[C@H](Nc2nc([C@H]3CC[C@H](NC(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O]
or a pharmaceutically acceptable salt thereof.

15. A method of treating and/or preventing a disease or disorder in a patient in need thereof, the method comprising: administering to the patient a therapeutically effective amount of the compound of claim 1 and an anti-cotinine antibody, or antigen-binding fragment thereof, wherein the disease or disorder is selected from a cancer, an inflammatory disease, an autoimmune disease, a viral infection, or a bacterial infection.

16. The method of claim 15, wherein the disease or disorder is mediated by chemokine receptor 2 (CCR2) and/or is associated with CCR2-positive pathogenic cells.

17. The method of claim 15, wherein the disease is a cancer that is a solid tumor.

18. The method of claim 15, wherein the disease or disorder is a cancer selected from leukemia, lymphoma, non-small cell lung cancer (NSCLC), hepatocellular carcinoma (HCC), colorectal cancer (CRC), cervical squamous cell carcinoma (CESC), head and neck squamous cell carcinoma (HNSC), pancreatic cancer, metastatic castration-resistant prostate cancer (mCRPC), ovarian cancer, endometrial cancer, bladder cancer, or breast cancer.

19. (canceled)

20. (canceled)

21. A method of increasing antibody-dependent cell cytotoxicity (ADCC) of C—C motif chemokine receptor 2 (CCR2)-expressing cells, the method comprising: contacting the cells with an effective amount of the compound of claim 1 and an anti-cotinine antibody, or antigen-binding fragment thereof, wherein the CCR2-binding moiety of the compound binds the CCR2 expressed on the cells.

22. A method of depleting C—C motif chemokine receptor 2 (CCR2)-expressing cells, the method comprising: contacting the cells with an effective amount of the compound of claim 1 and an anti-cotinine antibody, or antigen-binding fragment thereof, wherein the CCR2-binding moiety of the compound binds the CCR2 expressed on the cells.

23. (canceled)

24. The method of claim 18, wherein the anti-cotinine antibody has a heavy chain and a light chain, the heavy chain comprising a CDR1 having SEQ ID NO: 1, a CDR2 having SEQ ID NO: 2, and a CDR3 having SEQ ID NO: 3, and the light chain comprising a CDR1 having SEQ ID NO: 4, a CDR2 having SEQ ID NO: 5, and a CDR3 having SEQ ID NO: 6.

25. The method of claim 18, wherein the anti-cotinine antibody has a heavy chain and a light chain, the heavy chain comprising a heavy chain variable region (VH) having SEQ ID NO: 7, and the light chain comprising a light chain variable region (VL) having SEQ ID NO: 8.

26. (canceled)

27. (canceled)

28. The method of claim 18, wherein the anti-cotinine antibody has a heavy chain comprising SEQ ID NO: 9 and a light chain comprising SEQ ID NO: 10.

29. A combination comprising the compound of claim 1 and an anti-cotinine antibody, or antigen-binding fragment thereof.

30. The combination of claim 29, wherein the anti-cotinine antibody has a heavy chain and a light chain, the heavy chain comprising a CDR1 having SEQ ID NO: 1, a CDR2 having SEQ ID NO: 2, and a CDR3 having SEQ ID NO: 3, and the light chain comprising a CDR1 having SEQ ID NO: 4, a CDR2 having SEQ ID NO: 5, and a CDR3 having SEQ ID NO: 6.

31. The combination of claim 29, wherein the anti-cotinine antibody has a heavy chain and a light chain, the heavy chain comprising a heavy chain variable region (VH) having SEQ ID NO: 7, and the light chain comprising a light chain variable region (VL) having SEQ ID NO: 8.

32-34. (canceled)
